(19) United States
(12) Patent Application Publication
(10) Pub. No.: US 2025/0003009 A1
(43) Pub. Date: Jan. 2, 2025

(54) FRAGMENT CONSENSUS METHODS FOR ULTRASENSITIVE DETECTION OF ABERRANT METHYLATION

(71) Applicant: Foundation Medicine, Inc., Boston, MA (US)

(72) Inventors: Neil PETERMAN, San Francisco, CA (US); Alexander De Jong ROBERTSON, Princeton, NJ (US); Nicole Jacinda LAMBERT, San Diego, CA (US)

(73) Assignee: Foundation Medicine, Inc., Boston, MA (US)

(21) Appl. No.: 18/709,782

(22) PCT Filed: Nov. 18, 2022
(86) PCT No.: PCT/US2022/080181

§ 371 Date: May 13, 2024

Related U.S. Application Data
(60) Provisional application No. 63/281,574, filed on Nov. 19, 2021.

(51) Int. Cl. C12Q 1/6886

(52) U.S. Cl. CPC C12Q 1/6886; C12Q 2600/154

(57) ABSTRACT

Provided herein are methods related to detecting DNA methylation (e.g., level of methylation at CpG dinucleotide cluster(s)), as well as methods of treatment, uses, systems, and computer readable storage media related thereto. These methods allow for detection of aberrant DNA methylation patterns with low background and increased signal-to-background ratio, which can be useful, inter alia, in the early detection or monitoring of cancer.

### CROSS-REFERENCE TO RELATED APPLICATIONS

This application claims the benefit of U.S. Provisional Application No. 63/281,574, filed Nov. 19, 2021, which is hereby incorporated by reference in its entirety.

### FIELD

Provided herein are methods related to detecting methylation levels, as well as methods of diagnosis, prognosis, monitoring, screening, and treatment, as well as systems and computer-readable storage media related thereto.

### BACKGROUND

Aberrant methylation is widespread in cancer and can be detected in many different types of patient samples, including those that comprise cell-free DNA (cfDNA) or circulating cell-free DNA (ccfDNA). Detection of rare cancer-driven patterns is a key challenge for many liquid biopsy applications including detection and monitoring of minimal residual disease (MRD).

Some methylation patterns in cancer are associated with or predictive of response to particular treatment regimens or disease management strategies. For example, in glioblastoma, promoter methylation in the gene MGMT has been associated with better outcomes (Lalezari et al. (2013) Neuro Oncol 15:370-381). Methylation-based studies could lead to discovery of new predictive biomarkers to guide therapy and drug development. Many late-stage cancer patients have higher levels of cancer signal in ccfDNA; however, some patients have lower levels of cancer signal in ccfDNA and could benefit from ultrasensitive detection of methylation levels. In addition, late-stage patients with the best response to treatment (chemotherapy, immunotherapy, targeted therapy, or some combination) have dramatic reduction of cancer signal observed in successive ccfDNA samples just a few weeks into treatment (see, e.g., Davis, A. A. et al. (2020) Mol. Cancer Ther. 19:1486-1496; Hrebien, S. et al. (2019) Ann. Oncol. 30:945-952). Ultrasensitive detection of methylation levels may be useful, e.g., to continually monitor this subset of patients and detect recurrence as early as possible.

In early-stage cancers, ccfDNA often contains cancer-derived molecules at a frequency of 1 in 1,000 down to 1 in 100,000, presenting an obstacle to the application of many analytical methods. A similar challenge arises using other sample types where cancer DNA is present but at low quantities, including urine cell-free DNA, cerebrospinal fluid, and others. Sensitive detection of cancer signal at this level is likely necessary for the successful application of ccfDNA to detection of MRD and blood-based monitoring of early-stage cancer patients.

Measuring DNA methylation has been investigated as a way to detect cancer and distinguish tumor DNA from normal DNA, but existing methods have been found to be insufficient in enabling ultra-sensitive detection of cancer signals and improving analytical performance. Guo et al. (Nat. Genet. 2017 49:635-642) applied the concept of linkage disequilibrium to methylation and defined several read-based metrics to aid in detection and clustering of cancer in tissue and ccfDNA samples. These included Methyl-Haplotype Load, a score that rewards consecutively methylated or consecutively unmethylated sites. Liu et al. (Ann. Oncol. 2020 31:745-759) defined a concept of Methyl Variants, i.e., a set of 5 contiguous CG dinucleotides that are 0% or 100% methylated at high frequency in at least one known cancer sample (tissue biopsy) out of a dataset produced from a large cohort.

Therefore, there remains a need for improved methods and systems that provide robust and sensitive detection of aberrant methylation patterns in tumor DNA, as compared to normal DNA, with low background signal and increased signal-to-background ratio.

All references cited herein, including patent applications and publications, are incorporated by reference in their entirety.

### SUMMARY OF THE INVENTION

The present disclosure provides, inter alia, methods of detecting methylation level (and changes thereto) with extremely high sensitivity. These are based at least in part on the data disclosed herein demonstrating detection of cancer-associated changes in methylation with extremely high sensitivity and dramatically increased signal-to-background ratio, allowing the detection of very small amounts of nucleic acids with aberrant methylation in samples with overwhelmingly larger amounts of normal nucleic acids. These may find use, e.g., in detecting methylation levels as well as detection, monitoring, screening, diagnosis, and/or prognosis of cancer, or response to cancer treatment(s).

In one aspect, provided herein is a method of detecting methylation level (e.g., one or more of a methylation level or an unmethylation level) of a cluster of two or more CpG dinucleotides (e.g., in a sample from a subject), comprising: obtaining a plurality of nucleic acid fragments from the sample; amplifying the plurality of nucleic acid fragments; sequencing, by a sequencer, the plurality of nucleic acid fragments to obtain a plurality of sequence reads, wherein at least the plurality of amplified nucleic acid fragments has undergone cytosine conversion, and wherein the plurality of nucleic acid fragments corresponds to a genomic locus comprising a cluster of two or more CpG dinucleotides; determining, by a processor, a consensus methylation pattern for the cluster, wherein the consensus methylation pattern represents each CpG dinucleotide in the cluster for which methylation was detected in at least one sequence read from the plurality of sequence reads based on the cytosine conversion; generating, by a processor, a cluster consensus fraction (CCF) for the cluster, wherein the CCF represents a fraction of sequence reads corresponding to the cluster that show the consensus methylation pattern out of a total number of sequence reads from the plurality corresponding to the cluster; detecting one or more of the methylation level or the unmethylation level of the cluster based on the CCF; and generating a genomic profile for the subject based at least in part on the detected methylation level, the detected unmethylation level, or both. In one aspect, provided herein is a method of detecting methylation level (e.g., one or more of a methylation level or an unmethylation level) of a cluster of two or more CpG dinucleotides (e.g., in a sample from a subject), comprising: obtaining a plurality of nucleic acid fragments from a sample; amplifying the plurality of nucleic acid fragments; sequencing, by a sequencer, the plurality of amplified nucleic acid fragments to obtain a plurality of sequence reads, wherein at least the plurality of amplified nucleic acid fragments has undergone cytosine conversion, and wherein the plurality of nucleic acid fragments corresponds to a genomic locus comprising a cluster of two or more CpG dinucleotides; determining, by a processor, a consensus unmethylation pattern for the cluster, wherein the consensus unmethylation pattern represents each CpG dinucleotide in the cluster for which methylation was not detected based on the cytosine conversion in at least one sequence read from the plurality of sequence reads; generating, by a processor, a cluster consensus fraction (CCF) for the cluster, wherein the CCF represents a fraction of sequence reads corresponding to the cluster that show the consensus unmethylation pattern out of a total number of sequence reads from the plurality corresponding to the cluster; detecting one or more of the methylation level or the unmethylation level of the cluster based on the CCF; and generating a genomic profile for the subject based on the detected methylation level, the detected unmethylation level, or both.

In some embodiments according to any of the embodiments described herein, the CCF is at or above a threshold or reference value, and the method further comprises: detecting presence of cancer nucleic acids in the plurality of nucleic acid fragments, based at least in part on the CCF being at or above the threshold or reference value. In some embodiments according to any of the embodiments described herein, the CCF is below a threshold or reference value, and the method further comprises: detecting absence of cancer nucleic acids in the plurality of nucleic acid fragments, based at least in part on the CCF being below the threshold or reference value. In some embodiments according to any of the embodiments described herein, the CCF is at or above a threshold or reference value, and the method further comprises: detecting absence of cancer nucleic acids in the plurality of nucleic acid fragments, based at least in part on the CCF being at or above the threshold or reference value. In some embodiments according to any of the embodiments described herein, the CCF is below a threshold or reference value, and the method further comprises: detecting presence of cancer nucleic acids in the plurality of nucleic acid fragments, based at least in part on the CCF being below the threshold or reference value. In some embodiments, the method further comprises determining a consensus methylation pattern and CCF for more than one cluster. In some embodiments, the more than one cluster corresponds to more than one genomic locus. In some embodiments, the method further comprises determining a consensus methylation pattern and CCF for more than 1,000 clusters, between 10 and 100,000 clusters, or up to 1 million clusters. In some embodiments, the plurality of sequence reads comprises between 1 and 5 sequence reads, at least 100 sequence reads, or at least 1000 sequence reads corresponding to the cluster. In some embodiments, at least one CpG dinucleotide in the cluster is unmethylated in the consensus methylation pattern. In some embodiments, at least one CpG dinucleotide in the cluster is methylated in the consensus methylation pattern. In some embodiments, at least one cluster comprises two or more CpG dinucleotides. In some embodiments, each cluster comprises two or more CpG dinucleotides. In some embodiments, at least one cluster comprises five or more CpG dinucleotides. In some embodiments, each cluster comprises five or more CpG dinucleotides. In some embodiments, at least one cluster comprises six or more CpG dinucleotides. In some embodiments, all sites in the cluster except one are unmethylated in the consensus methylation pattern. In some embodiments, all sites in the cluster except two are unmethylated in the consensus methylation pattern. In some embodiments, at most 1 site, at most 2 sites, at most 10% of sites, at most 25% of sites, greater than 25% of sites, greater than 50% of sites, or greater than 75% of sites in the cluster is/are methylated in the consensus methylation pattern. In some embodiments, all sites in the cluster except one are methylated in the consensus methylation pattern. In some embodiments, all sites in the cluster except two are methylated in the consensus methylation pattern. In some embodiments, at most 1 site, at most 2 sites, at most 10% of sites, at most 25% of sites, greater than 25% of sites, greater than 50% of sites, or greater than 75% of sites in the cluster is/are unmethylated in the consensus methylation pattern.

In some embodiments according to any of the embodiments described herein, the plurality of sequence reads is obtained from whole-genome methyl sequencing (WGMS) or next-generation sequencing (NGS). In some embodiments, the plurality of sequence reads includes paired-end sequence reads. In some embodiments, the consensus methylation pattern and CCF are determined based on paired-end sequence reads corresponding to the cluster. In some embodiments, the plurality of sequence reads includes unpaired sequence reads. In some embodiments, the method further comprises prior to determining the consensus methylation pattern and CCF, demultiplexing sequence reads from the plurality of sequence reads. In some embodiments, the method further comprises prior to determining the consensus methylation pattern and CCF, performing three-letter alignment of sequence reads from the plurality to a reference genome. In some embodiments, the method further comprises prior to determining the consensus methylation pattern and CCF, excluding sequencing reads from the plurality that failed to undergo cytosine conversion. In some embodiments, the method further comprises prior to determining the consensus methylation pattern and CCF, excluding sequence reads with a base other than cytosine or thymine at a first position of at least one of the CpG dinucleotides. In some embodiments, the method further comprises prior to determining the consensus methylation pattern and CCF, excluding sequence reads with a base quality below a threshold base quality. In some embodiments, the consensus methylation pattern and CCMF are determined based on sequence reads that cover a plurality of CpG dinucleotides in the cluster. In some embodiments, the consensus methylation pattern and CCF are determined based on sequence reads that cover at least 50% of, at least 90% of, or all CpG dinucleotides in the cluster.

In some embodiments according to any of the embodiments described herein, the plurality of nucleic acid fragments has undergone cytosine conversion by bisulfite treatment, TET-assisted bisulfite treatment, TET-assisted pyridine borane treatment, oxidative bisulfite treatment, or APOBEC treatment. In some embodiments, the method further comprises prior to providing the plurality of sequence reads, treating a plurality of nucleic acids or nucleic acid fragments with bisulfite. In some embodiments, the method further comprises prior to providing the plurality of sequence reads, treating a plurality of nucleic acids or nucleic acid fragments with TET-assisted bisulfite treatment, TET-assisted pyridine borane treatment, oxidative bisulfite treatment, or APOBEC treatment. In some embodiments, the method further comprises prior to providing the plurality of sequence reads, subjecting a plurality of nucleic acids to fragmentation. In some embodiments, the method further comprises prior to providing the plurality of sequence reads, selectively enriching for a plurality of nucleic acids or nucleic acid fragments corresponding to a genomic locus that comprises a cluster of two or more CpG dinucleotides to produce an enriched sample. In some embodiments, the method further comprises prior to providing the plurality of sequence reads, amplifying a plurality of nucleic acids or nucleic acid fragments by polymerase chain reaction (PCR). In some embodiments, the method further comprises prior to providing the plurality of sequence reads, isolating a plurality of nucleic acids from a sample. In some embodiments, the sample comprises tumor cells and/or tumor nucleic acids. In some embodiments, the sample further comprises non-tumor cells and/or non-tumor nucleic acids. In some embodiments, the sample comprises a fraction of tumor nucleic acids that is less than 1%, less than 0.1%, and/or at least 0.01% of total nucleic acids. In some embodiments, the sample comprises tumor cell-free DNA (cfDNA), circulating cell-free DNA (ccfDNA), or circulating tumor DNA (ctDNA). In some embodiments, the sample comprises fluid, cells, or tissue. In some embodiments, the sample comprises blood or plasma. In some embodiments, the sample comprises a tumor biopsy or a circulating tumor cell. In some embodiments, the sample is a tissue sample, and the method further comprises: subjecting a plurality of nucleic acid molecules in the tissue to fragmentation to create the plurality of nucleic acid fragments. In some embodiments, the method further comprises ligating one or more adapters onto one or more nucleic acid fragments from the plurality of nucleic acid fragments prior to amplifying the plurality of nucleic acid fragments.

In another aspect, provided herein is a method of detecting cancer in an individual, comprising detecting the methylation level or the unmethylation level according to the method of any one of the above embodiments in a sample comprising a plurality of nucleic acids obtained from the individual, wherein the methylation level or the unmethylation level detected in the sample identifies the individual as having cancer.

In another aspect, provided herein is a method of screening an individual suspected of having cancer, comprising detecting the methylation level or the unmethylation level according to the method of any one of the above embodiments in a sample comprising a plurality of nucleic acids obtained from the individual, wherein the methylation level or the unmethylation level detected in the sample identifies the individual as likely to have cancer.

In another aspect, provided herein is a method of determining prognosis of an individual having cancer, comprising detecting the methylation level or the unmethylation level according to the method of any one of the above embodiments in a sample comprising a plurality of nucleic acids obtained from the individual, wherein the methylation level or the unmethylation level detected in the sample determines at least in part the prognosis of the individual.

In another aspect, provided herein is a method of predicting survival of an individual having cancer, comprising detecting the methylation level or the unmethylation level according to the method of any one of the above embodiments in a sample comprising a plurality of nucleic acids obtained from the individual, wherein the methylation level or the unmethylation level detected in the sample predicts at least in part the survival of the individual. In some embodiments, the methylation level detected in the sample is higher than a threshold or reference value, and wherein survival of the individual is predicted to be decreased, as compared to survival of an individual whose sample has a methylation level lower than the threshold or reference value.

In another aspect, provided herein is a method of predicting tumor burden of an individual having cancer, comprising detecting the methylation level or the unmethylation level according to the method of any one of the above embodiments in a sample comprising a plurality of nucleic acids obtained from the individual, wherein the methylation level or the unmethylation level detected in the sample predicts at least in part the tumor burden of the individual. In some embodiments, the methylation level detected in the sample is higher than a threshold or reference value, and wherein tumor burden of the individual is predicted to be increased, as compared to tumor burden of an individual whose sample has a methylation level lower than the threshold or reference value.

In another aspect, provided herein is a method of predicting responsiveness to treatment of an individual having cancer, comprising detecting the methylation level or the unmethylation level according to the method of any one of the above embodiments in a sample comprising a plurality of nucleic acids obtained from the individual, wherein the methylation level or the unmethylation level detected in the sample is used at least in part to predict responsiveness of the individual to a treatment.

In another aspect, provided herein is a method of identifying an individual having cancer who may benefit from a treatment comprising anthracycline-based chemotherapy, the method comprising detecting the methylation level or the unmethylation level according to the method of any one of the above embodiments in a sample comprising a plurality of nucleic acids obtained from the individual, wherein the plurality of nucleic acids includes one or more nucleic acids corresponding to a PITX2 locus, wherein methylation of the PITX2 locus detected in the sample identifies the individual as one who may benefit from the treatment comprising anthracycline-based chemotherapy.

In another aspect, provided herein is a method of selecting a therapy for an individual having cancer, the method comprising detecting the methylation level or the unmethylation level according to the method of any one of the above embodiments in a sample comprising a plurality of nucleic acids obtained from the individual, wherein the plurality of nucleic acids includes one or more nucleic acids corresponding to a PITX2 locus, wherein methylation of the PITX2 locus detected in the sample identifies the individual as one who may benefit from treatment comprising anthracycline-based chemotherapy.

In another aspect, provided herein is a method of identifying one or more treatment options for an individual having cancer, the method comprising: (a) detecting the methylation level or the unmethylation level according to the method of any one of the above embodiments in a sample comprising a plurality of nucleic acids obtained from the individual, wherein the plurality of nucleic acids includes one or more nucleic acids corresponding to a PITX2 locus; and (b) generating a report comprising one or more treatment options identified for the individual based at least in part on methylation of the PITX2 locus detected in the sample, wherein the one or more treatment options comprise anthracycline-based chemotherapy.

In another aspect, provided herein is a method of treating or delaying progression of cancer, comprising: (a) detecting the methylation level or the unmethylation level according to the method of any one of the above embodiments in a sample comprising a plurality of nucleic acids obtained from the individual, wherein the plurality of nucleic acids includes one or more nucleic acids corresponding to a PITX2 locus; and (b) administering to the individual an effective amount of anthracycline-based chemotherapy.

In another aspect, provided herein is a method of identifying an individual having cancer who may benefit from a treatment comprising an alkylating agent, the method comprising detecting the methylation level or the unmethylation level according to the method of any one of the above embodiments in a sample comprising a plurality of nucleic acids obtained from the individual, wherein the plurality of nucleic acids includes one or more nucleic acids corresponding to an MGMT locus, wherein methylation of the MGMT locus detected in the sample identifies the individual as one who may benefit from the treatment comprising an alkylating agent.

In another aspect, provided herein is a method of selecting a therapy for an individual having cancer, the method comprising detecting the methylation level or the unmethylation level according to the method of any one of the above embodiments in a sample comprising a plurality of nucleic acids obtained from the individual, wherein the plurality of nucleic acids includes one or more nucleic acids corresponding to an MGMT locus, wherein methylation of the MGMT locus detected in the sample identifies the individual as one who may benefit from treatment comprising an alkylating agent.

In another aspect, provided herein is a method of identifying one or more treatment options for an individual having cancer, the method comprising: (a) detecting the methylation level or the unmethylation level according to the method of any one of the above embodiments in a sample comprising a plurality of nucleic acids obtained from the individual, wherein the plurality of nucleic acids includes one or more nucleic acids corresponding to an MGMT locus; and (b) generating a report comprising one or more treatment options identified for the individual based at least in part on methylation of the MGMT locus detected in the sample, wherein the one or more treatment options comprise an alkylating agent.

In another aspect, provided herein is a method of treating or delaying progression of cancer, comprising: (a) detecting the methylation level or the unmethylation level according to the method of any one of the above embodiments in a sample comprising a plurality of nucleic acids obtained from the individual, wherein the plurality of nucleic acids includes one or more nucleic acids corresponding to an MGMT locus; and (b) administering to the individual an effective amount of an alkylating agent.

In another aspect, provided herein is a method of monitoring response of an individual being treated for cancer, comprising: (a) administering a treatment to an individual having cancer; and (b) detecting the methylation level or the unmethylation level according to the method of any one of the above embodiments in a sample comprising a plurality of nucleic acids obtained from the individual after treatment, wherein the methylation level or the unmethylation level detected in the sample is used at least in part to monitor response to the treatment. In some embodiments, detection of a methylation level after treatment that is less than a methylation level prior to treatment, or less than a threshold or reference value, indicates that the individual has responded to treatment. In some embodiments, detection of a methylation level after treatment that is not greater than a methylation level prior to treatment, or less than a threshold or reference value, indicates that the individual has responded to treatment.

In another aspect, provided herein is a method of monitoring a cancer in an individual, comprising: detecting the methylation level or the unmethylation level according to the method of any one of the above embodiments in a first sample comprising a plurality of nucleic acids obtained from the individual; detecting the methylation level or the unmethylation level according to the method of any one of the above embodiments in a second sample comprising a plurality of nucleic acids obtained from the individual, wherein the second sample is obtained from the individual after the first sample; and determining a difference in methylation level between the first and second samples, thereby monitoring the cancer in the individual.

In another aspect, provided herein is a method of monitoring response of an individual being treated for cancer, comprising: detecting the methylation level or the unmethylation level according to the method of any one of the above embodiments in a first sample comprising a plurality of nucleic acids obtained from the individual; after the first sample is obtained from the individual, administering a treatment to the individual; detecting the methylation level or the unmethylation level according to the method of any one of the above embodiments in a second sample comprising a plurality of nucleic acids obtained from the individual, wherein the second sample is obtained from the individual after administration of the treatment; and determining a difference in methylation level between the first and second samples, thereby monitoring response of the individual to the treatment.

In another aspect, provided herein is a method of detecting one or more of a methylation level or an unmethylation level of a cluster of two or more CpG dinucleotides from a sample, comprising: obtaining a plurality of sequence reads from a plurality of nucleic acid fragments exhibiting cytosine conversion; determining, by a processor, a consensus methylation pattern for a cluster of two or more CpG dinucleotides at a locus, wherein the consensus methylation pattern represents each CpG dinucleotide in the cluster for which methylation was detected; generating, by a processor, a cluster consensus fraction (CCF) for the cluster, wherein the CCF represents a fraction of sequence reads corresponding to the cluster that show the consensus methylation pattern out of a total number of sequence reads from the plurality corresponding to the cluster; and detecting, by the processor, one or more of the methylation level or the unmethylation level of the cluster based on the CCF. In another aspect, provided herein is a method of detecting one or more of a methylation level or an unmethylation level of a cluster of two or more CpG dinucleotides, comprising: sequencing, by a sequencer, the plurality of nucleic acid fragments to obtain the plurality of sequence reads; determining, by a processor, a consensus methylation pattern for the cluster, wherein the consensus methylation pattern represents each CpG dinucleotide in the cluster for which methylation was detected; generating, by a processor, a cluster consensus fraction (CCF) for the cluster, wherein the CCF represents a fraction of sequence reads corresponding to the cluster that show the consensus methylation pattern out of a total number of sequence reads from the plurality corresponding to the cluster, thereby detecting one or more of the methylation level or the unmethylation level of the cluster; and detecting, by the processor, one or more of the methylation level or the unmethylation level of the cluster based on the CCF. In some embodiments, the consensus methylation pattern represents each CpG dinucleotide in the cluster for which methylation was detected based on the cytosine conversion in at least one sequence read from the plurality. In one aspect, provided herein is a method of detecting one or more of a methylation level or an unmethylation level of a cluster of two or more CpG dinucleotides from a sample, comprising: obtaining a plurality of sequence reads from a plurality of nucleic acid fragments exhibiting cytosine conversion; determining, by a processor, a consensus unmethylation pattern for a cluster of two or more CpG dinucleotides at a locus, wherein the consensus unmethylation pattern represents each CpG dinucleotide in the cluster for which methylation was not detected; generating, by a processor, a cluster consensus fraction (CCF) for the cluster, wherein the CCF represents a fraction of sequence reads corresponding to the cluster that show the consensus unmethylation pattern out of a total number of sequence reads from the plurality corresponding to the cluster; and detecting, by the processor, one or more of the methylation level or the unmethylation level of the cluster based on the CCF. In some embodiments, the CCF represents a fraction of sequence reads corresponding to the cluster that show the consensus unmethylation pattern out of a total number of sequence reads from the plurality corresponding to the cluster based on the cytosine conversion in at least one sequence read from the plurality of sequence reads.

In another aspect, provided herein is a system, comprising: one or more processors; and a memory configured to store one or more computer program instructions, wherein the one or more computer program instructions when executed by the one or more processors are configured to: determine, using the one or more processors, a consensus methylation pattern for a cluster of two or more CpG dinucleotides at a genomic locus, wherein the consensus methylation pattern represents each CpG dinucleotide in the cluster for which methylation was detected in at least one sequence read from a plurality of sequence reads obtained from a plurality of nucleic acid fragments that has undergone cytosine conversion; and generate, using the one or more processors, a cluster consensus fraction (CCF) for the cluster, wherein the CCF represents a fraction of sequence reads corresponding to the cluster that show the consensus methylation pattern out of a total number of sequence reads from the plurality corresponding to the cluster. In another aspect, provided herein is a system, comprising: one or more processors; and a memory configured to store one or more computer program instructions, wherein the one or more computer program instructions when executed by the one or more processors are configured to: determine, using the one or more processors, a consensus unmethylation pattern for a cluster of two or more CpG dinucleotides at a genomic locus, wherein the consensus unmethylation pattern represents each CpG dinucleotide in the cluster for which methylation was not detected in at least one sequence read from a plurality of sequence reads obtained from a plurality of nucleic acid fragments that has undergone cytosine conversion; and generate, using the one or more processors, a cluster consensus fraction (CCF) for the cluster, wherein the CCF represents a fraction of sequence reads corresponding to the cluster that show the consensus unmethylation pattern out of a total number of sequence reads from the plurality corresponding to the cluster.

In some embodiments according to any of the embodiments described herein, the CCF is at or above a threshold or reference value, and wherein the one or more computer program instructions when executed by the one or more processors are further configured to: detect, using the one or more processors, presence of cancer nucleic acids in the plurality of nucleic acid fragments, based at least in part on the CCF being at or above the threshold or reference value. In some embodiments according to any of the embodiments described herein, the CCF is below a threshold or reference value, and wherein the one or more computer program instructions when executed by the one or more processors are further configured to: detect, using the one or more processors, absence of cancer nucleic acids in the plurality of nucleic acid fragments, based at least in part on the CCF being below the threshold or reference value. In some embodiments according to any of the embodiments described herein, the CCF is at or above a threshold or reference value, and wherein the one or more computer program instructions when executed by the one or more processors are further configured to: detect, using the one or more processors, absence of cancer nucleic acids in the plurality of nucleic acid fragments, based at least in part on the CCF being at or above the threshold or reference value. In some embodiments according to any of the embodiments described herein, the CCF is below a threshold or reference value, and wherein the one or more computer program instructions when executed by the one or more processors are further configured to: detect, using the one or more processors, presence of cancer nucleic acids in the plurality of nucleic acid fragments, based at least in part on the CCF being below the threshold or reference value. In some embodiments, the one or more computer program instructions when executed by the one or more processors are further configured to: determine, using the one or more processors, a consensus methylation pattern for more than one cluster of two or more CpG dinucleotides; and generate, using the one or more processors, a cluster consensus fraction (CCF) for more than one cluster. In some embodiments, the more than one cluster corresponds to more than one genomic locus. In some embodiments, the one or more computer program instructions when executed by the one or more processors are configured to determine a consensus methylation pattern and generate a CCF for more than 1,000, between 10 and 100,000, or up to 1 million clusters. In some embodiments, the one or more computer program instructions when executed by the one or more processors are further configured to, prior to determining the consensus methylation pattern and generating the CCF: demultiplex, using the one or more processors, sequence reads from the plurality of sequence reads. In some embodiments, the one or more computer program instructions when executed by the one or more processors are further configured to, prior to determining the consensus methylation pattern and generating the CCF: perform, using the one or more processors, three-letter alignment of sequence reads from the plurality to a reference genome. In some embodiments, the one or more computer program instructions when executed by the one or more processors are further configured to, prior to determining the consensus methylation pattern and generating the CCF: exclude, using the one or more processors, sequencing reads from the plurality that failed to undergo cytosine conversion. In some embodiments, the one or more computer program instructions when executed by the one or more processors are further configured to, prior to determining the consensus methylation pattern and generating the CCF: exclude, using the one or more processors, sequence reads with a base other than cytosine or thymine at a first position of at least one of the CpG dinucleotides. In some embodiments, the one or more computer program instructions when executed by the one or more processors are further configured to, prior to determining the consensus methylation pattern and generating the CCF: exclude, using the one or more processors, sequence reads with a base quality below a threshold base quality.

In another aspect, provided herein is a non-transitory computer readable storage medium comprising one or more programs executable by one or more computer processors for performing a method, comprising: obtaining a plurality of sequence reads from a plurality of nucleic acid fragments exhibiting cytosine conversion; determining, using the one or more processors, a consensus methylation pattern for a cluster of two or more CpG dinucleotides at a genomic locus, wherein the consensus methylation pattern represents each CpG dinucleotide in the cluster for which methylation was detected in at least one sequence read from a plurality of sequence reads; generating, using the one or more processors, a cluster consensus fraction (CCF) for the cluster, wherein the CCF represents a fraction of sequence reads corresponding to the cluster that show the consensus methylation pattern out of a total number of sequence reads from the plurality corresponding to the cluster; and detecting, by the processor, one or more of the methylation level or the unmethylation level of the cluster based on the CCF. In another aspect, provided herein is a non-transitory computer readable storage medium comprising one or more programs executable by one or more computer processors for performing a method, comprising: obtaining a plurality of sequence reads from a plurality of nucleic acid fragments exhibiting cytosine conversion; determining, using the one or more processors, a consensus unmethylation pattern for a cluster of two or more CpG dinucleotides at a locus, wherein the consensus unmethylation pattern represents each CpG dinucleotide in the cluster for which methylation was not detected in at least one sequence read from a plurality of sequence reads; generating, using the one or more processors, a cluster consensus fraction (CCF) for the cluster, wherein the CCF represents a fraction of sequence reads corresponding to the cluster that show the consensus unmethylation pattern out of a total number of sequence reads from the plurality corresponding to the cluster; and detecting, by the processor, one or more of a methylation level or an unmethylation level of the cluster based on the CCF.

In some embodiments according to any of the embodiments described herein, the plurality of sequence reads is obtained from a plurality of nucleic acid fragments that has undergone cytosine conversion. In some embodiments, the CCF is at or above a threshold or reference value, and wherein the method further comprises: detecting, using the one or more processors, presence of cancer nucleic acids in the plurality of nucleic acid fragments, based at least in part on the CCF being at or above the threshold or reference value. In some embodiments according to any of the embodiments described herein, the CCF is at or above a threshold or reference value, and wherein the method further comprises: detecting, using the one or more processors, absence of cancer nucleic acids in the plurality of nucleic acid fragments, based at least in part on the CCF being below the threshold or reference value. In some embodiments, the CCF is at or above a threshold or reference value, and wherein the method further comprises: detecting, using the one or more processors, absence of cancer nucleic acids in the plurality of nucleic acid fragments, based at least in part on the CCF being at or above the threshold or reference value. In some embodiments according to any of the embodiments described herein, the CCF is at or above a threshold or reference value, and wherein the method further comprises: detecting, using the one or more processors, presence of cancer nucleic acids in the plurality of nucleic acid fragments, based at least in part on the CCF being below the threshold or reference value. In some embodiments, the method further comprises: determining, using the one or more processors, a consensus methylation pattern for more than one cluster of two or more CpG dinucleotides; and generating, using the one or more processors, a cluster consensus fraction (CCF) more than one cluster. In some embodiments, the more than one cluster corresponds to more than one genomic locus. In some embodiments, the method comprises determining a consensus methylation pattern and generating a CCF for more than 1,000 clusters, between 10 and 100,000 clusters, or up to 1 million clusters. In some embodiments, the method comprises, prior to determining the consensus methylation pattern and generating the CCF: demultiplexing, using the one or more processors, sequence reads from the plurality of sequence reads. In some embodiments, the method comprises, prior to determining the consensus methylation pattern and generating the CCF: performing, using the one or more processors, three-letter alignment of sequence reads from the plurality to a reference genome. In some embodiments, the method comprises, prior to determining the consensus methylation pattern and generating the CCF: excluding, using the one or more processors, sequencing reads from the plurality that failed to undergo cytosine conversion. In some embodiments, the method comprises, prior to determining the consensus methylation pattern and generating the CCF: excluding, using the one or more processors, sequence reads with a base other than cytosine or thymine at a first position of at least one of the CpG dinucleotides. In some embodiments, the method comprises, prior to determining the consensus methylation pattern and generating the CCF: excluding, using the one or more processors, sequence reads with a base quality below a threshold base quality.

In some embodiments according to any of the embodiments described herein, the plurality of sequence reads comprises between 1 and 5 sequence reads, at least 100 sequence reads, or at least 1000 sequence reads corresponding to the cluster. In some embodiments, at least one CpG dinucleotide in the cluster is unmethylated in the consensus methylation pattern. In some embodiments, at least one CpG dinucleotide in the cluster is methylated in the consensus methylation pattern. In some embodiments, at least one cluster comprises two or more CpG dinucleotides. In some embodiments, each cluster comprises two or more CpG dinucleotides. In some embodiments, at least one cluster comprises five or more CpG dinucleotides. In some embodiments, each cluster comprises five or more CpG dinucleotides. In some embodiments, at least one cluster comprises six or more CpG dinucleotides. In some embodiments, all sites in the cluster except one are unmethylated in the consensus methylation pattern. In some embodiments, all sites in the cluster except two are unmethylated in the consensus methylation pattern. In some embodiments, at most 1 site, at most 2 sites, at most 10% of sites, at most 25% of sites, greater than 25% of sites, greater than 50% of sites, or greater than 75% of sites in the cluster is/are methylated in the consensus methylation pattern. In some embodiments, the plurality of sequence reads is obtained from whole-genome methyl sequencing (WGMS) or next-generation sequencing (NGS). In some embodiments, the plurality of sequence reads includes paired-end sequence reads. In some embodiments, the consensus methylation pattern and CCF are determined based on paired-end sequence reads corresponding to the cluster. In some embodiments, the plurality of sequence reads includes unpaired sequence reads. In some embodiments, the consensus methylation pattern and CCF are determined and generated based on sequence reads that cover a plurality of CpG dinucleotides in the cluster. In some embodiments, the consensus methylation pattern and CCF are determined based on sequence reads that cover at least 50% of, at least 90% of, or all CpG dinucleotides in the cluster. In some embodiments, the plurality of nucleic acid fragments has undergone cytosine conversion by bisulfite treatment, TET-assisted bisulfite treatment, TET-assisted pyridine borane treatment, oxidative bisulfite treatment, or APOBEC treatment.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1A provides a schematic diagram of an Average Methylation Fraction (AMF) approach for assessing DNA methylation.

FIG. 1B provides a schematic diagram of a Cluster Consensus Fraction (CCF) approach for assessing DNA methylation, according to some embodiments.

FIG. 2 shows the design of a cell line panel for identifying features to be used in whole-genome methylation sequencing of healthy and TNBC cell lines.

FIG. 3A shows the results of CCF analysis of hypermethylated clusters in 4 cancer cell lines, compared to negative control.

FIG. 3B shows the results of Cluster Consensus Unmethylation Fraction (CCUF) analysis of hypomethylated clusters in 4 cancer cell lines, compared to negative control.

FIGS. 4A-4C compare analysis of methylation using CCF approach (FIGS. 4A & 4B) vs. using AMF approach (FIG. 4C) in mixtures of cancer and healthy cells. CCF led to values consistently well above background for mixtures with fraction of cancer cells as low as 10′, whereas using AMF led to these mixtures having a signal at or below background.

FIG. 5 shows the sensitivity (at 95% specificity) of methylation detection by CCF as a function of the number of clusters selected for analysis, using indicated mixtures of cancer vs. healthy cells (from 1% down to 0.01% cancer cells).

FIG. 6 shows that aberrant methylation was correlated in control sample measurements.

FIG. 7 shows a comparison of methylation fractions obtained by AMF or majority methylation fraction approaches from sequencing TNBC cell lines or healthy cells (NA12878).

FIG. 8 depicts a block diagram of an exemplary process for detecting methylation level using CCF, in accordance with some embodiments.

FIG. 9 depicts a block diagram of an exemplary process for detecting cancer (e.g., tumor nucleic acids from a sample) using CCF, in accordance with some embodiments

FIG. 10 depicts an exemplary system, in accordance with some embodiments.

FIG. 11 depicts an exemplary device, in accordance with some embodiments.

### DETAILED DESCRIPTION

The present disclosure relates generally to detecting methylation level, e.g., of a cluster of CpG dinucleotides.

Aberrant methylation is a feature of many cancers and can be detected in many different types of patient samples, including those containing cell-free DNA (cfDNA) or circulating cell-free DNA (ccfDNA). Detection of rare cancer-driven methylation patterns is a key challenge in cancer screening and monitoring of minimal residual disease (MRD). The present disclosure describes, inter alia, methods for detecting aberrant methylation (e.g., DNA methylation in CpG dinucleotide clusters) that effectively reduce background and increase signal-to-background ratio, thus allowing for detection of very low-frequency tumor DNA in otherwise normal DNA samples, which may assist in early detection and/or monitoring of cancer.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “cancerous” refer to or describe the physiological condition in mammals that is typically characterized by unregulated cell growth. Included in this definition are benign and malignant cancers.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2-0-methyl-, 2-0-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“amidate”), P(O)R, P(0)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or aralkyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “detection” includes any means of detecting, including direct and indirect detection.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes), or by aberrant DNA methylation level and/or pattern).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations or DNA methylation level and/or pattern in a biological sample from an individual.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of a compound (e.g., an antagonist) or a pharmaceutical composition (e.g., a pharmaceutical composition including an antagonist) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker (e.g., DNA methylation) described herein. In certain embodiments, the manufacture or kit is promoted, distributed, or sold as a unit for performing the methods described herein.

The term “methylation” is used herein to refer to presence of a methyl group at the C5 position of a cytosine nucleotide within DNA nucleic acids (unless context indicates otherwise). This term includes 5-methylcytosine (5mC) as well as cytosine nucleotides in which the methyl group is further modified, such as 5-hydroxymethylcytosine (5hmC). This term also includes DNA nucleic acids that have been subjected to chemical or enzymatic conversion of nucleotides, such as bisulfite conversion that deaminates unmodified cytosines to uracil.

The term “aberrant methylation” is used herein to refer to a pattern of methylation that is not typically present in a normal tissue. For example, the term can refer to increased methylation at a site that is not normally methylated in a normal tissue, or decreased methylation at a site that is normally methylated in a normal tissue. In some embodiments, nucleic acids derived from a cancer cell (e.g., cancer nucleic acids) are characterized by aberrant methylation when their pattern and/or amount of methylation at one or more genomic loci differs from what is normally present at the corresponding locus/loci in a particular type of tissue.

The term “CpG dinucleotide” is used herein to refer to a region of 2 or more DNA bases in which a cytosine nucleotide is followed by a guanine nucleotide in the 5′->3′ direction, e.g., 5′-C-phosphate-G-3′. In many genomes, CpG dinucleotides can often be found in “clusters” or regions of DNA containing multiple CpG dinucleotides (also termed “CpG islands”). Much or most of DNA methylation in many genomes is present in CpG dinucleotides (in which the cytosine is methylated or hydroxymethylated).

### III. Methods, Systems, and Devices

Certain aspects of the present disclosure relate to methods of detecting methylation level (e.g., of a cluster of two or more CpG dinucleotides). In some embodiments, the methods comprise obtaining a plurality of nucleic acid fragments from a sample (e.g., from a subject); amplifying the plurality of nucleic acid fragments; sequencing, by a sequencer, the plurality of amplified nucleic acid fragments to obtain a plurality of sequence reads, wherein at least the plurality of amplified nucleic acid fragments has undergone cytosine conversion, and wherein the plurality of nucleic acid fragments corresponds to a genomic locus comprising a cluster of two or more CpG dinucleotides; determining, by a processor, a consensus methylation pattern for the cluster, wherein the consensus methylation pattern represents each CpG dinucleotide in the cluster for which methylation was detected based on the cytosine conversion in at least one sequence read from the plurality of sequence reads; generating, by a processor, a cluster consensus fraction (CCF) for the cluster, wherein the CCF represents a fraction of sequence reads corresponding to the cluster that show the consensus methylation pattern out of a total number of sequence reads from the plurality corresponding to the cluster; detecting one or more of the methylation level or the unmethylation level of the cluster based on the CCF; and generating a genomic profile for the subject based at least in part on the detected methylation level, the detected unmethylation level, or both.

In some embodiments, the methods comprise sequencing (e.g., by a sequencer) a plurality of nucleic acid fragments to obtain a plurality of sequence reads, wherein the plurality of nucleic acid fragments has undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster.

In other embodiments, the methods comprise sequencing (e.g., by a sequencer) a plurality of nucleic acid fragments to obtain a plurality of sequence reads, wherein the plurality of nucleic acid fragments has undergone cytosine conversion, and wherein the plurality of nucleic acid fragments corresponds to a genomic locus comprising a cluster of two or more CpG dinucleotides; determining (e.g., by a processor) a consensus unmethylation pattern for the cluster, wherein the consensus unmethylation pattern represents each CpG dinucleotide in the cluster for which methylation was not detected in at least one sequence read from the plurality of sequence reads based on the cytosine conversion; and generating (e.g., by a processor) a cluster consensus unmethylation fraction (CCUF) for the cluster, wherein the CCUF represents a fraction of sequence reads corresponding to the cluster that show the consensus unmethylation pattern out of a total number of sequence reads from the plurality corresponding to the cluster. It will be appreciated by those skilled in the art that the methods disclosed herein for measuring methylation (e.g., CCMF) could also be applied to measuring un- or non-methylated sites (e.g., CCUF) as well. It will be understood that the cluster consensus methylation fraction, the cluster consensus unmethylation fraction, or both may be generally referred to as a cluster consensus fraction (CCF)

Other aspects of the present disclosure relate to methods of detecting cancer in an individual, comprising detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, a CCF at or above a threshold or reference value indicates presence of cancer in the individual and identifies the individual as having cancer. In some embodiments, a CCF below a threshold or reference value does not indicate presence of cancer in the individual and identifies the individual as not having cancer. In some embodiments, the methods may find use, e.g., in screening for cancer (e.g., a new diagnosis in an individual that has not previously been diagnosed with cancer, or the same type of cancer) or monitoring the individual for recurrence or minimal residual disease (e.g., in an individual that has previously been diagnosed with cancer and achieved remission).

Other aspects of the present disclosure relate to methods of screening an individual suspected of having cancer, comprising detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, a CCF at or above a threshold or reference value indicates presence of cancer in the individual and identifies the individual as likely to have cancer. In some embodiments, a CCF below a threshold or reference value does not indicate presence of cancer in the individual and identifies the individual as likely not to have cancer. In some embodiments, the methods may find use, e.g., in screening for cancer (e.g., a new diagnosis in an individual that has not previously been diagnosed with cancer, or the same type of cancer) or monitoring the individual for recurrence or minimal residual disease (e.g., in an individual that has previously been diagnosed with cancer and achieved remission).

Other aspects of the present disclosure relate to methods of determining prognosis of an individual having cancer, comprising detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, a CCF at or above a threshold or reference value indicates presence of cancer in the individual and determines at least in part a prognosis of the individual. In some embodiments, a CCF below a threshold or reference value does not indicate presence of cancer in the individual and determines at least in part a prognosis of the individual. In some embodiments, a CCF at or above a threshold or reference value corresponds to poorer prognosis of an individual, as compared to that of an individual with a CCF below the threshold or reference value.

Other aspects of the present disclosure relate to methods of predicting survival of an individual having cancer, comprising detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, a CCF at or above a threshold or reference value indicates presence of cancer in the individual and predicts at least in part the survival of the individual. In some embodiments, a CCF below a threshold or reference value does not indicate presence of cancer in the individual and predicts at least in part the survival of the individual. In some embodiments, a CCF at or above a threshold or reference value corresponds to shorter survival of an individual, as compared to that of an individual with a CCF below the threshold or reference value. In some embodiments, the methylation level detected in the sample is higher than a threshold or reference value, and survival of the individual is predicted to be decreased, as compared to survival of an individual whose sample has a methylation level lower than the threshold or reference value.

Other aspects of the present disclosure relate to methods of predicting tumor burden of an individual having cancer, comprising detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, a CCF at or above a threshold or reference value predicts a higher tumor burden in the individual, as compared to a CCF below the threshold or reference value. In some embodiments, the methylation level detected in the sample is higher than a threshold or reference value, and tumor burden of the individual is predicted to be increased, as compared to tumor burden of an individual whose sample has a methylation level lower than the threshold or reference value.

Other aspects of the present disclosure relate to methods of predicting responsiveness to treatment of an individual having cancer, comprising detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, methylation level detected in the sample is used at least in part to predict responsiveness of the individual to a treatment.

Other aspects of the present disclosure relate to methods of monitoring response of an individual being treated for cancer, comprising administering a treatment to an individual having cancer, and detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, methylation level detected in the sample is used at least in part to monitor response to the treatment. In some embodiments, detection of a methylation level or CCF after treatment that is less than a methylation level or CCF prior to treatment, or less than a threshold or reference value, indicates that the individual has responded to treatment. In some embodiments, detection of a methylation level or CCF after treatment that is not greater than a methylation level or CCF prior to treatment, or less than a threshold or reference value, indicates that the individual has responded to treatment.

Other aspects of the present disclosure relate to methods of monitoring a cancer in an individual, comprising detecting methylation level (e.g., of a cluster of two or more CpG dinucleotides) according to any one of the methods of the present disclosure in a first sample obtained from the individual, detecting methylation level (e.g., of a cluster of two or more CpG dinucleotides) according to any one of the methods of the present disclosure in a second sample obtained from the individual after the first sample, and determining a difference in methylation level or CCF between the first and second samples. In some embodiments, the methods comprise sequencing (e.g., by a sequencer) a plurality of nucleic acid fragments to obtain a plurality of sequence reads, wherein the plurality of nucleic acid fragments is obtained from the first sample from the individual and has subsequently undergone cytosine conversion, and wherein the plurality of nucleic acid fragments corresponds to a genomic locus comprising a cluster of two or more CpG dinucleotides; sequencing (e.g., by a sequencer) a second plurality of nucleic acid fragments to obtain a second plurality of sequence reads, wherein the second plurality of nucleic acid fragments is obtained from the second sample from the individual and has subsequently undergone cytosine conversion, and wherein the second plurality of nucleic acid fragments corresponds to a genomic locus comprising a cluster of two or more CpG dinucleotides; determining (e.g., by a processor) a second consensus methylation pattern for the cluster, wherein the second consensus methylation pattern represents each CpG dinucleotide in the cluster for which methylation was detected in at least one sequence read from the second plurality of sequence reads based on the cytosine conversion; generating (e.g., by a processor) a second cluster consensus fraction (CCF) for the cluster, wherein the second CCF represents a fraction of sequence reads corresponding to the cluster that show the consensus methylation pattern out of a total number of sequence reads from the plurality corresponding to the cluster; and comparing the first and second CCFs. In some embodiments, a second CCF that is greater than the first CCF indicates progression, spread, or expansion of the cancer. In some embodiments, a second CCF that is less than the first CCF indicates regression, response to treatment, or decrease of the cancer. In some embodiments, a second CCF that is equal to the first CCF indicates lack of progression or stability of the cancer.

Other aspects of the present disclosure relate to methods of monitoring response of an individual being treated for cancer, comprising detecting methylation level (e.g., of a cluster of two or more CpG dinucleotides) according to any one of the methods of the present disclosure in a first sample obtained from the individual, administering a treatment to the individual, detecting methylation level (e.g., of a cluster of two or more CpG dinucleotides) according to any one of the methods of the present disclosure in a second sample obtained from the individual after administration of the treatment and the first sample, and determining a difference in methylation level between the first and second samples. In some embodiments, the methods comprise sequencing (e.g., by a sequencer) a plurality of nucleic acid fragments to obtain a plurality of sequence reads, wherein the plurality of nucleic acid fragments is obtained from the first sample from the individual and has subsequently undergone cytosine conversion, and wherein the plurality of nucleic acid fragments corresponds to a genomic locus comprising a cluster of two or more CpG dinucleotides; sequencing (e.g., by a sequencer) a second plurality of nucleic acid fragments to obtain a second plurality of sequence reads, wherein the second plurality of nucleic acid fragments is obtained from the second sample from the individual and has subsequently undergone cytosine conversion, and wherein the second plurality of nucleic acid fragments corresponds to a genomic locus comprising a cluster of two or more CpG dinucleotides; determining (e.g., by a processor) a second consensus methylation pattern for the cluster, wherein the second consensus methylation pattern represents each CpG dinucleotide in the cluster for which methylation was detected in at least one sequence read from the second plurality of sequence reads based on the cytosine conversion; generating (e.g., by a processor) a second cluster consensus fraction (CCF) for the cluster, wherein the second CCF represents a fraction of sequence reads corresponding to the cluster that show the consensus methylation pattern out of a total number of sequence reads from the plurality corresponding to the cluster; and comparing the first and second CCFs. In some embodiments, a second CCF that is greater than the first CCF indicates lack of response to treatment. In some embodiments, a second CCF that is less than the first CCF indicates response to treatment. In some embodiments, a second CCF that is equal to the first CCF indicates partial or stable response to treatment.

In some embodiments, the methods of the present disclosure further comprise (e.g., if the CCF is at or above a threshold or reference value): detecting presence of cancer nucleic acids in the plurality of nucleic acid fragments. In some embodiments, detection of cancer nucleic acids is based at least in part on the CCF being at or above the threshold or reference value. In some embodiments, the methods of the present disclosure further comprise (e.g., if the CCF is at or above a threshold or reference value): detecting presence of cancer in a sample.

In some embodiments, the methods of the present disclosure further comprise (e.g., if the CCF is below a threshold or reference value): detecting absence of cancer nucleic acids in the plurality of nucleic acid fragments. In some embodiments, detecting absence of cancer nucleic acids is based at least in part on the CCF being below the threshold or reference value. In some embodiments, the methods of the present disclosure further comprise (e.g., if the CCF is below a threshold or reference value): detecting absence of cancer in a sample. In some embodiments, the methods of the present disclosure further comprise (e.g., if the CCF is below a threshold or reference value): detecting presence of normal or wild-type nucleic acids in the plurality of nucleic acid fragments (e.g., nucleic acids such as DNA having normal or wild-type levels and/or patterns of methylation). In some embodiments, detecting presence of normal or wild-type nucleic acids is based at least in part on the CCF being below the threshold or reference value. In some embodiments, the methods of the present disclosure further comprise (e.g., if the CCF is below a threshold or reference value): detecting presence of normal/wild-type cells or methylation levels/pattern in a sample.

In some embodiments, the methods of the present disclosure comprise determining a consensus methylation pattern and/or CCF for more than one cluster (e.g., of two or more CpG dinucleotides). In some embodiments, the clusters correspond to more than one genomic locus. In some embodiments, the methods of the present disclosure comprise determining a consensus methylation pattern and/or CCF for more than 10 clusters, more than 50 clusters, more than 100 clusters, more than 200 clusters, more than 300 clusters, more than 400 clusters, more than 500 clusters, more than 600 clusters, more than 700 clusters, more than 800 clusters, more than 900 clusters, more than 1000 clusters, more than 2000 clusters, more than 3000 clusters, more than 4000 clusters, more than 5000 clusters, more than 6000 clusters, more than 7000 clusters, more than 8000 clusters, more than 9000 clusters, more than 10000 clusters, more than 20000 clusters, more than 30000 clusters, more than 40000 clusters, more than 50000 clusters, more than 60000 clusters, more than 70000 clusters, more than 80000 clusters, more than 90000 clusters, more than 100000 clusters, more than 200000 clusters, more than 300000 clusters, more than 400000 clusters, more than 500000 clusters, more than 600000 clusters, more than 700000 clusters, more than 800000 clusters, more than 900000 clusters, or up to 1000000 clusters (e.g., of two or more CpG dinucleotides). In some embodiments, the methods of the present disclosure comprise determining a consensus methylation pattern and/or CCF for between 10 and 100000 clusters, between 100 and 100000 clusters, between 1000 and 100000 clusters, between 10000 and 100000 clusters, between 10 and 100 clusters, between 10 and 1000 clusters, between 10 and 10000 clusters, or between 10 and 1000000 clusters (e.g., of two or more CpG dinucleotides). In some embodiments, the methods of the present disclosure comprise determining a consensus methylation pattern and/or CCF for a number of clusters (e.g., of two or more CpG dinucleotides) having an upper limit of 50, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 clusters, and an independently selected lower limit of 900000, 800000, 700000, 600000, 500000, 400000, 300000, 200000, 100000, 90000, 80000, 70000, 60000, 50000, 40000, 30000, 20000, 10000, 9000, 8000, 7000, 6000, 5000, 4000, 3000, 2000, 1000, 900, 800, 700, 600, 500, 400, 300, 200, 100, 50, or 10 clusters, wherein the upper limit is greater than the lower limit.

In some embodiments, the plurality of sequence reads comprises at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, or at least 5000 sequence reads corresponding to a cluster. In some embodiments, the plurality of sequence reads comprises between 1 and 5, between 1 and 10, between 1 and 20, between 1 and 30, between 1 and 40, between 1 and 50, between 1 and 100, between 10 and 100, between 10 and 1000, between 50 and 1000, or between 100 and 1000 sequence reads corresponding to a cluster. In some embodiments, the plurality of sequence reads comprises a number of sequence reads corresponding to a cluster having an upper limit of 5000, 4000, 3000, 2000, 1000, 900, 800, 700, 600, 500, 400, 300, 200, 100, 90, 80, 70, 60, 50, 40, 30, 20, 10, or 5, and an independently selected lower limit of 5, 10, 20, 30, 40, 50, 60, 70, 80, 90, 100, 200, 300, 400, 500, 600, 700, 800, 900, 1000, 2000, 3000, 4000, or 5000, wherein the upper limit is greater than the lower limit.

In some embodiments, at least one CpG dinucleotide in the cluster is unmethylated in the consensus methylation pattern. In some embodiments, at least one CpG dinucleotide in the cluster is methylated in the consensus methylation pattern. In some embodiments, at least one CpG dinucleotide in the cluster is unmethylated in the consensus unmethylation pattern. In some embodiments, at least one CpG dinucleotide in the cluster is methylated in the consensus unmethylation pattern.

In some embodiments, at least one cluster comprises two or more, three or more, four or more, five or more, six or more, seven or more, eight or more, nine or more, or ten or more CpG dinucleotides. In some embodiments, each cluster comprises two or more, three or more, four or more, five or more, six or more, seven or more, eight or more, nine or more, or ten or more CpG dinucleotides. In some embodiments, a cluster comprises two or more, three or more, four or more, five or more, six or more, seven or more, eight or more, nine or more, or ten or more CpG dinucleotides within a specified number of bases, e.g., within 300 bases or less, 250 bases or less, 200 bases or less, 150 bases or less, 125 bases or less, 100 bases or less, 90 bases or less, 80 bases or less, 70 bases or less, 60 bases or less, or 50 bases or less. In some embodiments, a cluster comprises two or more, three or more, four or more, five or more, six or more, seven or more, eight or more, nine or more, or ten or more CpG dinucleotides within 80 bases or less.

In some embodiments, all sites in the cluster except one, except two, except 5, or except 10 are unmethylated in the consensus methylation pattern. In some embodiments, all sites in the cluster except one, except two, except 5, or except 10 are unmethylated in the consensus unmethylation pattern.

In some embodiments, at most 1 site, at most 2 sites, at most 3 sites, at most 4 sites, at most 5 sites, or at most 10 sites in the cluster is/are methylated in the consensus methylation pattern. In some embodiments, at most 1 site, at most 2 sites, at most 3 sites, at most 4 sites, at most 5 sites, or at most 10 sites in the cluster is/are methylated in the consensus unmethylation pattern. In some embodiments, at most 5%, at most 10%, at most 20%, at most 25%, at most 30%, at most 40%, at most 50%, or at most 75% of sites in the cluster are methylated in the consensus methylation pattern. In some embodiments, at most 5%, at most 10%, at most 20%, at most 25%, at most 30%, at most 40%, at most 50%, or at most 75% of sites in the cluster are methylated in the consensus unmethylation pattern. In some embodiments, greater than 5%, greater than 10%, greater than 20%, greater than 25%, greater than 30%, greater than 40%, greater than 50%, or greater than 75% of sites in the cluster are methylated in the consensus methylation pattern. In some embodiments, greater than 5%, greater than 10%, greater than 20%, greater than 25%, greater than 30%, greater than 40%, greater than 50%, or greater than 75% of sites in the cluster are methylated in the consensus unmethylation pattern. In some embodiments, the percentage of sites in the cluster that are methylated in the consensus methylation pattern has an upper limit of 5%, 10%, 15%, 20%, 25%, 30%, 35%, 40%, 45%, 50%, 55%, 60%, 65%, 70%, 75%, 80%, 85%, 90%, or 95%, and an independently selected lower limit of 90%, 85%, 80%, 75%, 70%, 65%, 60%, 55%, 50%, 45%, 40%, 35%, 30%, 25%, 20%, 15%, 10%, 5%, or 1%, wherein the upper limit is greater than the lower limit. In some embodiments, the percentage of sites in the cluster that are methylated in the consensus unmethylation pattern has an upper limit of 5%, 10%, 15%, 20%, 25%, 30%, 35%, 40%, 45%, 50%, 55%, 60%, 65%, 70%, 75%, 80%, 85%, 90%, or 95%, and an independently selected lower limit of 90%, 85%, 80%, 75%, 70%, 65%, 60%, 55%, 50%, 45%, 40%, 35%, 30%, 25%, 20%, 15%, 10%, 5%, or 1%, wherein the upper limit is greater than the lower limit. In some embodiments, at most 1 site, at most 2 sites, at most 3 sites, at most 4 sites, at most 5 sites, or at most 10 sites in the cluster is/are unmethylated in the consensus methylation pattern. In some embodiments, at most 1 site, at most 2 sites, at most 3 sites, at most 4 sites, at most 5 sites, or at most 10 sites in the cluster is/are unmethylated in the consensus unmethylation pattern. In some embodiments, at most 5%, at most 10%, at most 20%, at most 25%, at most 30%, at most 40%, at most 50%, or at most 75% of sites in the cluster are unmethylated in the consensus methylation pattern. In some embodiments, at most 5%, at most 10%, at most 20%, at most 25%, at most 30%, at most 40%, at most 50%, or at most 75% of sites in the cluster are unmethylated in the consensus unmethylation pattern. In some embodiments, greater than 5%, greater than 10%, greater than 20%, greater than 25%, greater than 30%, greater than 40%, greater than 50%, or greater than 75% of sites in the cluster are unmethylated in the consensus methylation pattern. In some embodiments, greater than 5%, greater than 10%, greater than 20%, greater than 25%, greater than 30%, greater than 40%, greater than 50%, or greater than 75% of sites in the cluster are unmethylated in the consensus unmethylation pattern. In some embodiments, the percentage of sites in the cluster that are unmethylated in the consensus methylation pattern has an upper limit of 5%, 10%, 15%, 20%, 25%, 30%, 35%, 40%, 45%, 50%, 55%, 60%, 65%, 70%, 75%, 80%, 85%, 90%, or 95%, and an independently selected lower limit of 90%, 85%, 80%, 75%, 70%, 65%, 60%, 55%, 50%, 45%, 40%, 35%, 30%, 25%, 20%, 15%, 10%, 5%, or 1%, wherein the upper limit is greater than the lower limit. In some embodiments, the percentage of sites in the cluster that are unmethylated in the consensus unmethylation pattern has an upper limit of 5%, 10%, 15%, 20%, 25%, 30%, 35%, 40%, 45%, 50%, 55%, 60%, 65%, 70%, 75%, 80%, 85%, 90%, or 95%, and an independently selected lower limit of 90%, 85%, 80%, 75%, 70%, 65%, 60%, 55%, 50%, 45%, 40%, 35%, 30%, 25%, 20%, 15%, 10%, 5%, or 1%, wherein the upper limit is greater than the lower limit.

In some embodiments, consensus methylation pattern and/or CCF are determined based on sequence reads that cover a plurality of CpG dinucleotides in a cluster. In some embodiments, consensus unmethylation pattern and/or CCUF are determined based on sequence reads that cover a plurality of CpG dinucleotides in a cluster. In some embodiments, consensus methylation pattern and/or CCMF are determined based on sequence reads that cover at least 10%, at least 20%, at least 30%, at least 40%, at least 50%, at least 60%, at least 70%, at least 80%, or at least 90% of CpG dinucleotides in a cluster. In some embodiments, consensus unmethylation pattern and/or CCUF are determined based on sequence reads that cover at least 10%, at least 20%, at least 30%, at least 40%, at least 50%, at least 60%, at least 70%, at least 80%, or at least 90% of CpG dinucleotides in a cluster. In some embodiments, consensus methylation pattern and/or CCMF are determined based on sequence reads that cover all CpG dinucleotides in a cluster. In some embodiments, consensus unmethylation pattern and/or CCUF are determined based on sequence reads that cover all CpG dinucleotides in a cluster.

In some embodiments, an observed CCF (e.g., CCMF or CCUF) is compared to a threshold or reference value. In some embodiments, the threshold or reference value refers to a threshold or reference value used for comparison purposes. In some embodiments, the threshold or reference value is obtained from analyzing a wild-type or non-tumor sample or nucleic acid(s), e.g., a control sample, normal adjacent tumor (NAT), or any other non-cancerous sample from the same or a different individual. In some embodiments, the threshold or reference value is obtained from analyzing (e.g., averaging or any other type of statistical aggregation) values obtained from multiple samples or individuals. In some embodiments, the threshold or reference value refers to an intermediate value obtained by analyzing one or more cancer or tumor tissue/cells/nucleic acids and one or more normal, wild-type, or non-tumor tissue/cells/nucleic acids, such that the threshold or reference value indicates cancer and includes value(s) obtained from one or more cancer or tumor cells/nucleic acids, or indicates normal tissue/cells/nucleic acids and includes value(s) obtained from one or more normal, wild-type, or non-tumor tissue/cells/nucleic acids.

As is known in the art, methylation levels of particular genomic loci can be predictive of response to particular treatments, e.g., predictive biomarkers, and/or presence of particular types of cancer. See, e.g., Locke, W. J. et al. (2019) Front. Genet. 10:1150. For example, methylation of the MGMT locus (encoding an O-6-methylguanine-DNA methyltransferase) is thought to predict better response to alkylating agents such as temozolomide, and methlylation of the PITX2 locus (encoding a paired-like homeodomain 2 transcription factor) is thought to predict better response to anthracycline-based chemotherapy. As such, in some embodiments, the methods of the present disclosure are used to detect methylation level at particular genomic loci, e.g., in particular cancer types. In some embodiments, methylation of the MGMT locus is detected in glioblastoma. In some embodiments, methylation of the PITX2 locus is detected in breast cancer. In some embodiments, methylation of the TWIST1, ONECUT2, OTX1, SOX1, and/or IRAK3 loci is/are detected in bladder cancer. In some embodiments, methylation of the ASTN1, DLX1, ITGA4, RXFP3, SOX17, and/or ZNF671 loci is/are detected in cervical cancer. In some embodiments, methylation of the FAM19A4 and/or hsa-mir124-2 loci is/are detected in cervical cancer. In some embodiments, methylation of the NDRG4 and/or BMP3 loci is/are detected in colorectal cancer. In some embodiments, methylation of the VIM locus is detected in colorectal cancer. In some embodiments, methylation of the IKZF1 and/or BCAT1 loci is/are detected in colorectal cancer. In some embodiments, methylation of the SEPT9 locus is detected in colorectal cancer or hepatocellular carcinoma. In some embodiments, methylation of the SHOX2 and/or PTGER4 loci is/are detected in lung cancer. In some embodiments, methylation of the GSTP1, APC, and/or RASSF1 loci is/are detected in prostate cancer. Details of these genomic loci (e.g., human genomic loci) are known in the art. For example, see NCBI Gene ID No. 4255 for the human MGMT locus and NCBI Gene ID No. 5308 for the human PITX2 locus.

Other aspects of the present disclosure relate to methods of identifying an individual having cancer who may benefit from a treatment comprising anthracycline-based chemotherapy, comprising detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, the plurality of nucleic acids includes one or more nucleic acids corresponding to a PITX2 locus. In some embodiments, methylation of the PITX2 locus detected in the sample identifies the individual as one who may benefit from the treatment comprising anthracycline-based chemotherapy.

Other aspects of the present disclosure relate to methods of selecting a therapy for an individual having cancer, comprising detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, the plurality of nucleic acids includes one or more nucleic acids corresponding to a PITX2 locus. In some embodiments, methylation of the PITX2 locus detected in the sample identifies the individual as one who may benefit from treatment comprising anthracycline-based chemotherapy.

Other aspects of the present disclosure relate to methods of identifying one or more treatment options for an individual having cancer, comprising detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, the plurality of nucleic acids includes one or more nucleic acids corresponding to a PITX2 locus. In some embodiments, the methods further comprise generating a report comprising one or more treatment options identified for the individual based at least in part on methylation of the PITX2 locus detected in the sample. In some embodiments, the one or more treatment options comprise anthracycline-based chemotherapy.

Other aspects of the present disclosure relate to methods of treating or delaying progression of cancer, comprising detecting methylation level (e.g., of a cluster of two or more CpG dinucleotides) according to any one of the methods of the present disclosure and administering to the individual an effective amount of anthracycline-based chemotherapy. In some embodiments, detecting the methylation level comprises sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, the plurality of nucleic acids includes one or more nucleic acids corresponding to a PITX2 locus.

As is known in the art, anthracycline-based chemotherapies are part of a class of drugs that act broadly by intercalating into DNA, inhibiting DNA/RNA synthesis, generating reactive oxygen species, and blocking the activity of topoisomerase II. Examples of anthracycline-based chemotherapies include, but are not limited to, doxorubicin (Adriamycin®, Rubex®), daunorubicin (Cerubidine®, Vyxeos®, daunomycin), epirubicin (Ellence®, Pharmorubicin®), idarubicin (Idamycin®), and mitoxantrone (Novantrone®).

Other aspects of the present disclosure relate to methods of identifying an individual having cancer who may benefit from a treatment comprising an alkylating agent, comprising detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, the plurality of nucleic acids includes one or more nucleic acids corresponding to a MGMT locus. In some embodiments, methylation of the MGMT locus detected in the sample identifies the individual as one who may benefit from the treatment comprising an alkylating agent.

Other aspects of the present disclosure relate to methods of selecting a therapy for an individual having cancer, comprising detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, the plurality of nucleic acids includes one or more nucleic acids corresponding to a MGMT locus. In some embodiments, methylation of the MGMT locus detected in the sample identifies the individual as one who may benefit from treatment comprising an alkylating agent.

Other aspects of the present disclosure relate to methods of identifying one or more treatment options for an individual having cancer, comprising detecting methylation level (e.g., of a cluster of two or more CpG dinucleotides) according to any one of the methods of the present disclosure. In some embodiments, the methods comprise sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, the plurality of nucleic acids includes one or more nucleic acids corresponding to a MGMT locus. In some embodiments, the methods further comprise generating a report comprising one or more treatment options identified for the individual based at least in part on methylation of the MGMT locus detected in the sample. In some embodiments, the one or more treatment options comprise an alkylating agent.

Other aspects of the present disclosure relate to methods of treating or delaying progression of cancer, comprising detecting methylation level (e.g., of a cluster of two or more CpG dinucleotides) according to any one of the methods of the present disclosure and administering to the individual an effective amount of an alkylating agent. In some embodiments, detecting the methylation level comprises sequencing (e.g., by a sequencer) a plurality of nucleic acid fragments to obtain a plurality of sequence reads, wherein the plurality of nucleic acid fragments is obtained from a sample from the individual and has subsequently undergone cytosine conversion, and wherein the plurality of nucleic acid fragments corresponds to a genomic locus comprising a cluster of two or more CpG dinucleotides; determining (e.g., by a processor) a consensus methylation pattern for the cluster, wherein the consensus methylation pattern represents each CpG dinucleotide in the cluster for which methylation was detected in at least one sequence read from the plurality of sequence reads based on the cytosine conversion; and generating (e.g., by a processor) a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, the plurality of nucleic acids includes one or more nucleic acids corresponding to a MGMT locus.

As is known in the art, alkylating agents refer to a broad group of chemicals that react with biological molecules to form covalent bonds, either directly (SN1) or via a reactive intermediate (SN2). Classes of alkylating agents include, but are not limited to, nitrogen mustards (e.g., mechlorethamine, mechlorethamine oxide hydrochloride, cyclophosphamide, cholophosphamide, chlomaphazine, bendamustine, estramustine, ifosfamide, melphalan, novembichin, phenesterine, prednimustine, trofosfamide, chlorambucil, and uracil mustard), aziridines (e.g., benzodopa, carboquone, meturedopa, uredopa, thiotepa, mitomycin C, and diaziquone (AZQ)), epoxides (e.g., dianhydrogalactitol and dibromodulcitol), alkyl sulfonates (e.g., busulfan, hepsulfam, improsulfan, and piposulfan), nitrosoureas (e.g., carmustine, lomustine, chlorozotocin, semustine or methyl CCNU, numustine, ranimnustine, streptozocin, and fotemustine), triazenes/hydrazines (e.g., procarbazine, dacarbazine or DTIC, methylazoxyprocarbazine, temozolomide), and methylamelamines/ethylenimines (e.g., hexamethylmelamine, altretamine, triethylenemelamine, trietylenephosphoramide, triethiylenethiophosphoramide, trimethylolomelamine, altretamine, and thiotepa).

### Detection of Methylation

Certain aspects of the present disclosure relate to methods of detecting methylation level (e.g., of a cluster of two or more CpG dinucleotides) of a plurality of nucleic acid fragments, e.g., DNA fragments.

CpG dinucleotides or sites typically refer to regions of DNA where a cytosine nucleotide is located immediately adjacent to a guanine nucleotide in the linear sequence. “CpG” refers to cytosine and guanine separated by a phosphate (i.e., —C-phosphate-G-). Regions of the DNA that have a higher frequency or concentration of CpG sites are known as “CpG islands”. Many genes in mammalian genomes have CpG islands associated with the transcriptional start site (including the promoter) of the gene, which play a pivotal role in controlling gene expression. See, e.g., US PG Pub. No. US20140357497. Aberrant methylation patterns are observed in many types of cancer. For example, in normal tissue, CpG islands are often unmethylated but a subset of islands becomes methylated during oncogenesis, cellular development, and various disease states. Hypermethylation (i.e. an increased level of methylation) of CpG sites within the promoters of genes can lead to their silencing, a feature found, e.g., in a number of human cancers (for example the silencing of tumor suppressor genes).

In some embodiments, the plurality of nucleic acid fragments has undergone cytosine conversion. A commonly-used method of determining the methylation level and/or pattern of DNA requires methylation status-dependent conversion of cytosine in order to distinguish between methylated and non-methylated CpG dinucleotide sequences. For example, methylation of CpG dinucleotide sequences can be measured by employing cytosine conversion based technologies, which rely on methylation status-dependent chemical modification of CpG sequences within isolated genomic DNA, or fragments thereof, followed by DNA sequence analysis. Chemical reagents that are able to distinguish between methylated and non-methylated CpG dinucleotide sequences include hydrazine, which cleaves the nucleic acid, and bisulfite treatment. Bisulfite treatment followed by alkaline hydrolysis specifically converts non-methylated cytosine to uracil, leaving 5-methylcytosine unmodified as described by Olek A., Nucleic Acids Res. 24:5064-6, 1996 or Frommer et al., Proc. Natl. Acad. Sci. USA 89:1827-1831 (1992). The bisulfite-treated DNA can subsequently be analyzed by conventional molecular techniques, such as PCR amplification, sequencing, and detection comprising oligonucleotide hybridization. See, e.g., U.S. Pat. No. 10,174,372.

Various methodologies for cytosine conversion are known in the art. In some embodiments, a plurality of nucleic acids or nucleic acid fragments of the present disclosure has undergone cytosine conversion by bisulfite treatment, TET-assisted bisulfite treatment, TET-assisted pyridine borane treatment, oxidative bisulfite treatment, or APOBEC treatment, e.g., prior to sequencing, determining a consensus methylation or unmethylation pattern, and generating a CCMF or CCUF.

As such, in some embodiments, the methods of the present disclosure comprise treating a plurality of nucleic acids or nucleic acid fragments of the present disclosure with bisulfite. Bisulfite sequencing is a commonly used method in the art for generating methylation data at single-base resolution. Bisulfite conversion or treatment refers to a biochemical process for converting unmethylated cytosine residue to uracil or thymine residues (e.g., deamination to uracil, followed by amplification as thymine during PCR), whereby methylated cytosine residues (e.g., 5-methylcytosine, 5mC; or 5-hydroxymethylcytosine, 5hmC) are preserved. Reagents to convert cytosine to uracil are known to those of skill in the art and include bisulfite reagents such as sodium bisulfite, potassium bisulfite, ammonium bisulfite, magnesium bisulfite, sodium metabisulfite, potassium metabisulfite, ammonium metabisulfite, magnesium metabisulfite and the like.

In some embodiments, the methods of the present disclosure comprise treating a plurality of nucleic acids or nucleic acid fragments of the present disclosure with enzymatic digestion and bisulfite treatment. The principle of the method is that the fragmentation of DNA is not achieved by ultrasound but achieved by combined enzymatic digestion by multiple endonucleases (MseI, Tsp 509I, NlaIII and Hpy CH4V), wherein the restriction enzyme cutting sites of MseI, Tsp509I, NlaIII and Hpy CH4V are TTAA, AATT, CATG and TGCA, respectively. See, e.g., Smiraglia D J, et al. Oncogene 2002; 21: 5414-5426. This is followed by bisulfite treatment, e.g., as described herein.

Enzymatic methods for cytosine conversion are also known, e.g., enzymatic methyl sequencing (EM-seq). Such approaches can be advantageous because they employ enzymes instead of bisulfite, which can damage and fragment DNA, leading to DNA loss and potentially biased sequencing. For example, TET2 (the Ten-eleven translocation (Tet) family 2 methylcytosine dioxygenase) and T4-BGT (T4 phage beta-glucosyltransferase) can be used to convert 5mC and 5hmC into products that cannot be deaminated by APOBEC3A (apolipoprotein B mRNA editing enzyme, catalytic polypeptide-like 3A), then APOBEC3A is used to deaminate unmodified cytosines by converting them into uracils. See, e.g., Vaisvila, R. et al. (2021) Genome Res. 31:1-10.

In some embodiments, the methods of the present disclosure comprise treating a plurality of nucleic acids or nucleic acid fragments of the present disclosure with TET-assisted bisulfite (e.g., TAB-seq). In the TAB-seq approach, beta-glucosyltransferase (βGT) is used to convert 5hmC into β-glucosyl-5-hydroxymethylcytosine (5gmC), and a Tet enzyme (e.g., mTet1) is used to oxidize 5mC into 5-carboxylcytosine (5caC). Subsequently, nucleic acids can be treated with bisulfite. See, e.g., Yu, M. et al. (2018) Methods Mol. Biol. 1708:645-663.

In some embodiments, the methods of the present disclosure comprise treating a plurality of nucleic acids or nucleic acid fragments of the present disclosure with TET-assisted pyridine borane (e.g., TAPS). In the TAPS approach, a TET methylcytosine dioxygenase is used to oxidize 5mC and 5hmC into 5caC, then 5caC is reduced into dihydrouracil (DHU) via pyridine borane. DHU is converted to thymine during subsequent PCR. See, e.g., Liu, Y. et al. (2019) Nat. Biotechnol. 37:424-429.

In some embodiments, the methods of the present disclosure comprise treating a plurality of nucleic acids or nucleic acid fragments of the present disclosure with oxidative bisulfite (e.g., oxBS). In the oxBS approach, 5hmC is oxidized into 5-formylcytosine (5fC), which can be converted to uracil under bisulfite. Sequencing results from bisulfite vs. oxidative bisulfite treatment can then be used to infer 5hmC levels from 5mC. See, e.g., Booth, M. J. et al. (2013) Nat. Protocols 8:1841-1851. This approach can be scaled on a genome-wide level in oxBS-seq; see, e.g., Kirschner, K. et al. (2018) Methods Mol. Biol. 1708:665-678.

In some embodiments, the methods of the present disclosure comprise treating a plurality of nucleic acids or nucleic acid fragments of the present disclosure with APOBEC. Enzymatic reagents to convert cytosine to uracil, i.e. cytosine deaminases, include those of the APOBEC family, such as APOBEC-seq or APOBEC3A. The APOBEC family members are cytidine deaminases that convert cytosine to uracil while maintaining 5-methyl cytosine, i.e. without altering 5-methyl cytosine. Such enzymes are described in US2013/0244237 and WO2018165366 and are commercially available (see, e.g., the NEBNext® Enzymatic Methyl-seq Kit, New England Biolabs). Non-limiting examples of APOBEC family proteins include APOBEC1, APOBEC2, APOBEC3A, APOBEC3B, APOBEC3C, APOBEC3D, APOBEC3F, APOBEC3G, APOBEC3H, APOBEC4, and Activation-induced (cytidine) deaminase.

### Sequencing

In some embodiments, a plurality of sequence reads of the present disclosure is obtained from whole-genome methyl sequencing (WGMS) or next-generation sequencing (NGS).

Various methods for WGMS are known in the art. Generally, these methods combine cytosine conversion (e.g., using the methods described supra) with whole-genome sequencing techniques. For example, in some embodiments, the WGMS comprises bisulfite sequencing, whole genome bisulfite sequencing (WGBS), APOBEC-seq, methyl-CpG-binding domain (MBD) protein capture, methyl-DNA immunoprecipitation (MeDIP-seq), methylation sensitive restriction enzyme sequencing (MSRE/MRE-Seq or Methyl-Seq), oxidative bisulfite sequencing (oxBS-Seq), reduced representative bisulfite sequencing (RRBS), or Tet-assisted bisulfite sequencing (TAB-Seq).

Some WGMS methods rely upon library construction and adapter ligation, followed by standard bisulfite conversion and sequencing (e.g., WGBS). Alternatively, bisulfite treatment can be carried out prior to adaptor ligation (see, e.g., Miura, F. et al. (2012) Nucleic Acids Res. 40:e136). More recent techniques use other cytosine conversion methods such as enzymatic approaches in order to reduce damage to DNA caused by bisulfite, e.g., as in the commercially available NEBNext® Enzymatic Methyl-seq Kit (New England Biolabs). Steps of library amplification, quantification, and sequencing generally follow bisulfite conversion. In some embodiments, prior to WGMS, nucleic acids are extracted from a sample. In some embodiments, prior to WGMS, nucleic acids are subjected to fragmentation, repair, and adaptor ligation. As noted previously, cytosine conversion can be carried out before or after adaptor ligation. In some embodiments, DNA repair is performed after cytosine conversion. PCR amplification (generally at least two cycles) is performed after cytosine conversion to convert uracils (generated by formerly unmethylated cytosines) into thymine, and is accomplished using a polymerase that is able to read uracil (excluding polymerases with proofreading and repair activities). In some embodiments, prior to sequencing, fragments are enriched for desired length. In some embodiments, prior to sequencing, nucleic acids are enriched for methylated sequences, such as by immunoprecipitation using an antibody specific for 5mC as in the MeDIP approach (see, e.g., Pomraning, K. R. et al. (2009) Methods 47:142-150.

NGS methods are known in the art, and are described, e.g., in Metzker, M. (2010) Nature Biotechnology Reviews 11:31-46. Platforms for next-generation sequencing include, e.g., Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, and Pacific Biosciences' PacBio RS system. NGS technologies can include one or more of steps, e.g., template preparation, sequencing and imaging, and data analysis. Methods for template preparation can include steps such as randomly breaking nucleic acids (e.g., genomic DNA) into smaller sizes and generating sequencing templates (e.g., fragment templates or mate-pair templates). The spatially separated templates can be attached or immobilized to a solid surface or support, allowing massive amounts of sequencing reactions to be performed simultaneously. Types of templates that can be used for NGS reactions include, e.g., clonally amplified templates originating from single DNA molecules, and single DNA molecule templates. Exemplary sequencing and imaging steps for NGS include, e.g., cyclic reversible termination (CRT), sequencing by ligation (SBL), single-molecule addition (pyrosequencing), and real-time sequencing. After NGS reads have been generated, they can be aligned to a known reference sequence or assembled de novo. For example, identifying genetic variations such as single-nucleotide polymorphism and structural variants in a sample (e.g., a tumor sample) can be accomplished by aligning NGS reads to a reference sequence (e.g., a wild type sequence). Methods of sequence alignment for NGS are described e.g., in Trapnell C. and Salzberg S. L. Nature Biotech., 2009, 27:455-457. Examples of de novo assemblies are described, e.g., in Warren R. et al., Bioinformatics, 2007, 23:500-501; Butler J. et al., Genome Res., 2008, 18:810-820; and Zerbino D. R. and Birney E., Genome Res., 2008, 18:821-829. Sequence alignment or assembly can be performed using read data from one or more NGS platforms, e.g., mixing Roche/454 and Illumina/Solexa read data. In some embodiments, NGS is performed according to the methods described in, e.g., Frampton, G. M. et al. (2013) Nat. Biotech. 31:1023-1031; and/or Montesion, M., et al., Cancer Discovery (2021) 11(2):282-92.

In some embodiments, the methods further comprise, prior to sequencing the plurality of polynucleotides or providing a plurality of sequence reads: subjecting a plurality of nucleic acids to fragmentation. A variety of DNA fragmentation techniques are used in the art prior to NGS or WGMS approaches. In some embodiments, nucleic acids are fragmented by nebulization, in which compressed gas is used to mechanically shear nucleic acids through a small opening. In some embodiments, nucleic acids are fragmented by sonication, in which ultrasonic waves are used to shear nucleic acids. In some embodiments, nucleic acids are fragmented enzymatically, e.g., using one or more enzymes to digest nucleic acids into fragments. See, e.g., the NEBNext® dsDNA Fragmentase, a mixture of two enzymes: one that randomly generates dsDNA nicks, and one that recognizes nicked sites and cuts the opposite strand, generating dsDNA breaks.

In some embodiments, the methods further comprise, prior to sequencing the plurality of polynucleotides or providing a plurality of sequence reads: selectively enriching for a plurality of nucleic acids or nucleic acid fragments corresponding to a genomic locus that comprises a cluster of two or more CpG dinucleotides to produce an enriched sample. For example, one or more baits or probes can be used to hybridize with a genomic locus of interest or fragment thereof, e.g., comprising a cluster of two or more CpG dinucleotides. See, e.g., Graham, B. I. et al. Twist Fast Hybridization targeted methylation sequencing: a tunable target enrichment solution for methylation detection [abstract]. In: Proceedings of the American Association for Cancer Research Annual Meeting 2021; 2021 Apr. 10-15 and May 17-21. Philadelphia (PA): AACR; Cancer Res 2021; 81(13_Suppl):Abstract nr 2098.

In some embodiments, the methods further comprise, prior to sequencing the plurality of polynucleotides or providing a plurality of sequence reads: amplifying a plurality of nucleic acids or nucleic acid fragments by polymerase chain reaction (PCR). A variety of PCR techniques suitable for WGMS and NGS are known in the art. As noted above, in some embodiments, a plurality of nucleic acids or nucleic acid fragments is amplified by PCR after cytosine conversion, and PCR amplification is used to convert uracils or other products of cytosine conversion into thymines. In some embodiments, the PCR amplification is performed using deoxyribonucleotides comprising thymine.

In some embodiments, the methods further comprise, prior to sequencing the plurality of polynucleotides or providing a plurality of sequence reads: contacting a mixture of polynucleotides with the bait molecule under conditions suitable for hybridization, wherein the mixture comprises a plurality of polynucleotides capable of hybridization with the bait molecule; and isolating a plurality of polynucleotides that hybridized with the bait molecule, wherein the isolated plurality of polynucleotides that hybridized with the bait molecule are sequenced by NGS.

In some embodiments, a plurality of sequence reads is obtained by performing sequencing on nucleic acids captured by hybridization with a bait molecule. In some embodiments, the plurality of sequence reads was obtained by performing whole exome sequencing on nucleic acids captured by hybridization with a bait molecule. In some embodiments, the plurality of sequence reads was obtained by performing next-generation sequencing (NGS), whole exome sequencing, or methylation sequencing (e.g., WGMS) on nucleic acids captured by hybridization with the bait molecule.

In some embodiments, a hybrid capture approach is used. Further details about this and other hybrid capture processes can be found in U.S. Pat. No. 9,340,830; Frampton, G. M. et al. (2013) Nat. Biotech. 31:1023-1031; and Montesion, M., et al., Cancer Discovery (2021) 11(2):282-92. In some embodiments, the methods further comprise, prior to contacting the mixture of polynucleotides with the bait molecule: obtaining a sample from an individual, wherein the sample comprises tumor cells and/or tumor nucleic acids; and extracting the mixture of polynucleotides from the sample, wherein the mixture of polynucleotides is from the tumor cells and/or tumor nucleic acids. In some embodiments, the sample further comprises non-tumor cells.

In some embodiments, a plurality of sequence reads of the present disclosure includes paired-end sequence reads. In some embodiments, consensus methylation pattern and/or CCF are determined based on paired-end sequence reads corresponding to one or more cluster(s). In some embodiments, consensus unmethylation pattern and/or CCUF are determined based on paired-end sequence reads corresponding to one or more cluster(s). Generally, paired-end sequencing methodologies are described, e.g., in WO2007/010252, WO2007/091077, and WO03/74734. This approach utilizes pairwise sequencing of a double-stranded polynucleotide template, which results in the sequential determination of nucleotide sequences in two distinct and separate regions of the polynucleotide template. The paired-end methodology makes it possible to obtain two linked or paired reads of sequence information from each double-stranded template on a clustered array, rather than just a single sequencing read as can be obtained with other methods. Paired end sequencing technology can make special use of clustered arrays, generally formed by solid-phase amplification, for example as set forth in WO03/74734. Target polynucleotide duplexes, fitted with adapters, are immobilized to a solid support at the 5′ ends of each strand of each duplex, for example, via bridge amplification as described above, forming dense clusters of double stranded DNA. Because both strands are immobilized at their 5′ ends, sequencing primers are then hybridized to the free 3′ end and sequencing by synthesis is performed. Adapter sequences can be inserted in between target sequences to allow for up to four reads from each duplex, as described in WO2007/091077. In a further adaptation of this methodology, specific strands can be cleaved in a controlled fashion as set forth in WO2007/010252. As a result, the timing of the sequencing read for each strand can be controlled, permitting sequential determination of the nucleotide sequences in two distinct and separate regions on complementary strands of the double-stranded template. See, e.g., U.S. Pat. No. 10,174,372.

In some embodiments, the plurality of sequence reads includes unpaired sequence reads.

In some embodiments, the methods of the present disclosure further comprise, prior to determining a consensus methylation pattern and CCF: demultiplexing sequence reads from a plurality of sequence reads. In some embodiments, the methods of the present disclosure further comprise, prior to determining a consensus methylation pattern and CCF: performing alignment of sequence reads from the plurality to a reference genome, e.g., a human reference genome. In some embodiments, the alignment is a three-letter alignment to a human reference genome. In some embodiments, the methods of the present disclosure further comprise, prior to determining a consensus methylation pattern and CCF: excluding sequencing reads from the plurality that failed to undergo cytosine conversion. In some embodiments, the methods of the present disclosure further comprise, prior to determining a consensus methylation pattern and CCF: excluding sequence reads with a base other than cytosine or thymine at a first position of at least one of the CpG dinucleotides. For example, these can be due to sequencing errors or mutations (somatic or germline). In some embodiments, the methods of the present disclosure further comprise, prior to determining a consensus methylation pattern and CCF: excluding sequence reads with a base quality below a threshold base quality. In some embodiments, base calls at a cytosine within a CpG dinucleotide are determined using two overlapping paired-end sequence reads.

### Samples and Cancers

In some embodiments, the methods of the present disclosure further comprise isolating a plurality of nucleic acids from a sample. In some embodiments, nucleic acids are obtained from a sample, e.g., comprising tumor cells and/or tumor nucleic acids. For example, the sample can comprise tumor cell(s), circulating tumor cell(s), tumor nucleic acids (e.g., tumor circulating tumor DNA, cfDNA, or cfRNA), part or all of a tumor biopsy, fluid, cells, tissue, mRNA, DNA, RNA, cell-free DNA, and/or cell-free RNA. In some embodiments, the sample is from a tumor biopsy or tumor specimen. In some embodiments, the sample further comprises non-tumor cells and/or non-tumor nucleic acids. In some embodiments, the fluid comprises blood, serum, plasma, saliva, semen, cerebral spinal fluid, amniotic fluid, peritoneal fluid, interstitial fluid, etc. In some embodiments, the sample further comprises non-tumor cells and/or non-tumor nucleic acids.

In some embodiments, the sample comprises a fraction of tumor nucleic acids that is less than 1% of total nucleic acids, less than 0.5% of total nucleic acids, less than 0.1% of total nucleic acids, or less than 0.05% of total nucleic acids. In some embodiments, the sample comprises a fraction of tumor nucleic acids that is at least 0.01%, at least 0.05%, or at least 0.1% of total nucleic acids. In some embodiments, the sample comprises a fraction of tumor nucleic acids having an upper limit of 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, or 0.02% of total nucleic acids and an independently selected lower limit of 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% of total nucleic acids, wherein the upper limit is greater than the lower limit. Advantageously, as demonstrated herein, the methods of the present disclosure allow for robust, ultrasensitive detection of aberrant methylation levels in slight amounts of tumor nucleic acids amongst otherwise normal nucleic acids.

In some embodiments, the sample is or comprises biological tissue or fluid. The sample can contain compounds that are not naturally intermixed with the tissue in nature such as preservatives, anticoagulants, buffers, fixatives, nutrients, antibiotics or the like. In one embodiment, the sample is preserved as a frozen sample or as a formaldehyde- or paraformaldehyde-fixed paraffin-embedded (FFPE) tissue preparation. For example, the sample can be embedded in a matrix, e.g., an FFPE block or a frozen sample. In another embodiment, the sample is a blood or blood constituent sample. In yet another embodiment, the sample is a bone marrow aspirate sample. In another embodiment, the sample comprises cell-free DNA (cfDNA) or circulating cell-free DNA (ccfDNA), e.g., tumor cfDNA or tumor ccfDNA. Without wishing to be bound by theory, it is believed that in some embodiments, cfDNA is DNA from apoptosed or necrotic cells. Typically, cfDNA is bound by protein (e.g., histone) and protected by nucleases. CfDNA can be used as a biomarker, for example, for non-invasive prenatal testing (NIPT), organ transplant, cardiomyopathy, microbiome, and cancer. In another embodiment, the sample comprises circulating tumor DNA (ctDNA). Without wishing to be bound by theory, it is believed that in some embodiments, ctDNA is cfDNA with a genetic or epigenetic alteration (e.g., a somatic alteration or a methylation signature) that can discriminate it originating from a tumor cell versus a non-tumor cell. In another embodiment, the sample comprises circulating tumor cells (CTCs). Without wishing to be bound by theory, it is believed that in some embodiments, CTCs are cells shed from a primary or metastatic tumor into the circulation. In some embodiments, CTCs apoptose and are a source of ctDNA in the blood/lymph.

In some embodiments of any of the methods provided herein, the cancer is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy. In some embodiments, the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer is appendix adenocarcinoma, bladder adenocarcinoma, bladder urothelial (transitional cell) carcinoma, breast cancer not otherwise specified (NOS), breast carcinoma NOS, breast invasive ductal carcinoma (IDC), breast invasive lobular carcinoma (ILC), cervix squamous cell carcinoma (SCC), colon adenocarcinoma (CRC), esophagus adenocarcinoma, esophagus carcinoma NOS, esophagus squamous cell carcinoma (SCC), eye intraocular melanoma, gallbladder adenocarcinoma, gastroesophageal junction adenocarcinoma, intra-hepatic cholangiocarcinoma, kidney cancer NOS, liver hepatocellular carcinoma (HCC), lung cancer NOS, lung adenocarcinoma, lung large cell carcinoma, lung non-small cell lung carcinoma (NSCLC) NOS, lung small cell undifferentiated carcinoma, lung squamous cell carcinoma (SCC), ovary cancer NOS, pancreas cancer NOS, pancreas ductal adenocarcinoma, pancreatobiliary carcinoma, prostate cancer NOS, prostate acinar adenocarcinoma, prostate ductal adenocarcinoma, rectum adenocarcinoma (CRC), skin melanoma, small intestine adenocarcinoma, soft tissue sarcoma NOS, stomach adenocarcinoma NOS, unknown primary cancer NOS, unknown primary adenocarcinoma, unknown primary carcinoma (CUP) NOS, unknown primary neuroendocrine tumor, unknown primary squamous cell carcinoma (SCC), or uterus endometrial adenocarcinoma NOS.

### Software, Systems, and Devices

In another aspect, provided herein are systems comprising a memory configured to store one or more program instructions; and one or more processors configured to execute the one or more program instructions. In some embodiments, the one or more program instructions when executed by the one or more processors are configured to: determine, using the one or more processors, a consensus methylation pattern for a cluster of two or more CpG dinucleotides at a genomic locus, wherein the consensus methylation pattern represents each CpG dinucleotide in the cluster for which methylation was detected in at least one sequence read from a plurality of sequence reads obtained from a plurality of nucleic acid fragments that has undergone cytosine conversion; and generate, using the one or more processors, a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, if the CCF is at or above a threshold or reference value, and the one or more computer program instructions are further configured to: detect, using the one or more processors, presence of cancer nucleic acids in the plurality of nucleic acid fragments, based at least in part on the CCF being at or above the threshold or reference value. In some embodiments, if the CCF is below a threshold or reference value, the one or more computer program instructions are further configured to: detect, using the one or more processors, absence of cancer nucleic acids in the plurality of nucleic acid fragments, based at least in part on the CCF being below the threshold or reference value. In some embodiments, the one or more computer program instructions are further configured to determine, using the one or more processors, a consensus methylation pattern for more than one cluster of two or more CpG dinucleotides; and generate, using the one or more processors, a cluster consensus fraction (CCF) for more than one cluster, e.g., according to any of the methods disclosed herein. In some aspects, provided herein are systems comprising a memory and one or more processors. In some embodiments, the memory comprises one or more programs for execution by the one or more processors, the one or more programs including instructions which, when executed by the one or more processors, cause the system to perform the method according to any of the embodiments described herein.

In another aspect, provided herein are transitory or non-transitory computer readable storage media. In some embodiments, the transitory or non-transitory computer readable storage media comprise one or more programs executable by one or more computer processors for performing a method. In some embodiments, the method comprises: determining, using the one or more processors, a consensus methylation pattern for a cluster of two or more CpG dinucleotides at a genomic locus, wherein the consensus methylation pattern represents each CpG dinucleotide in the cluster for which methylation was detected in at least one sequence read from a plurality of sequence reads obtained from a plurality of nucleic acid fragments that has undergone cytosine conversion; and generating, using the one or more processors, a cluster consensus fraction (CCF) for the cluster, wherein the CCF represents a fraction of sequence reads corresponding to the cluster that show the consensus methylation pattern out of a total number of sequence reads from the plurality corresponding to the cluster. In some embodiments, if the CCF is at or above a threshold or reference value, the method further comprises: detecting, using the one or more processors, presence of cancer nucleic acids in the plurality of nucleic acid fragments, based at least in part on the CCF being at or above the threshold or reference value. In some embodiments, if the CCF is at or above a threshold or reference value, the method further comprises: detecting, using the one or more processors, absence of cancer nucleic acids in the plurality of nucleic acid fragments, based at least in part on the CCF being below the threshold or reference value. In some embodiments, the method further comprises determining, using the one or more processors, a consensus methylation pattern for more than one cluster of two or more CpG dinucleotides; and generating, using the one or more processors, a cluster consensus fraction (CCF) more than one cluster, e.g., according to any of the methods disclosed herein. In some aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform the method according to any of the embodiments described herein.

FIG. 11 illustrates an example of a computing device in accordance with one embodiment. Device 1100 can be a host computer connected to a network. Device 1100 can be a client computer or a server. As shown in FIG. 11, device 1100 can be any suitable type of microprocessor-based device, such as a personal computer, workstation, server or handheld computing device (portable electronic device) such as a phone or tablet. The device can include, for example, one or more of processor(s) 1110, input device 1120, output device 1130, storage 1140, communication device 1160, power supply 1170, operating system 1180, and system bus 1190. Input device 1120 and output device 1130 can generally correspond to those described herein, and can either be connectable or integrated with the computer.

Input device 1120 can be any suitable device that provides input, such as a touch screen, keyboard or keypad, mouse, or voice-recognition device. Output device 1130 can be any suitable device that provides output, such as a touch screen, haptics device, or speaker.

Storage 1140 can be any suitable device that provides storage (e.g., an electrical, magnetic or optical memory including a RAM (volatile and non-volatile), cache, hard drive, or removable storage disk). Communication device 1160 can include any suitable device capable of transmitting and receiving signals over a network, such as a network interface chip or device. The components of the computer can be connected in any suitable manner, such as via a wired media (e.g., a physical bus, ethernet, or any other wire transfer technology) or wirelessly (e.g., Bluetooth®, Wi-Fi®, or any other wireless technology). For example, in FIG. 11, the components are connected by System Bus 1190.

Detection module 1150, which can be stored as executable instructions in storage 1140 and executed by processor(s) 1110, can include, for example, the processes that embody the functionality of the present disclosure (e.g., as embodied in the devices as described herein).

Detection module 1150 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1140, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Detection module 1150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

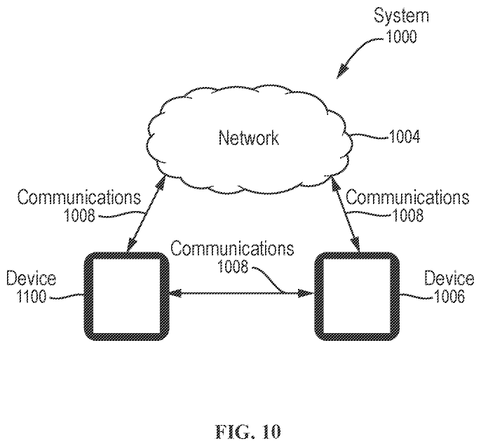

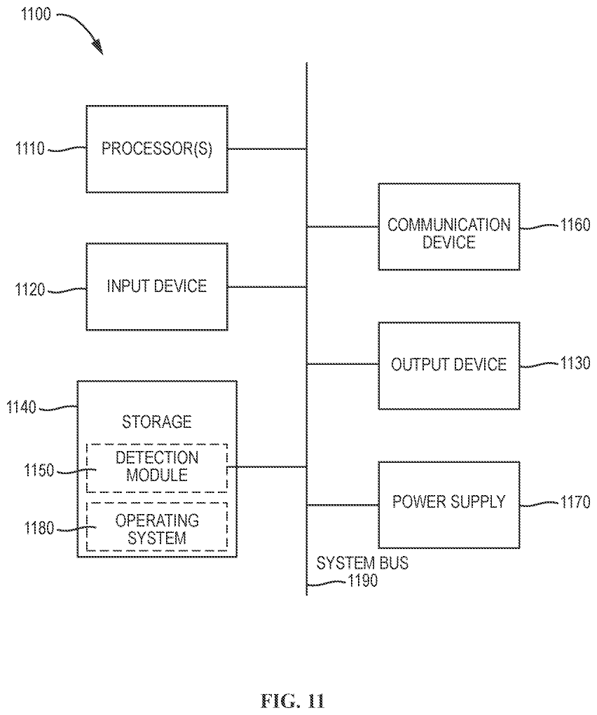

Device 1100 may be connected to a network (e.g., Network 1004, as shown in FIG. 10 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1100 can implement any operating system (e.g., Operating System 1180) suitable for operating on the network. Detection module 1150 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, Operating System 1180 is executed by one or more processors, e.g., Processor(s) 1110.

Device 1100 can further include Power Supply 1170, which can be any suitable power supply.

In some embodiments, Detection module 1150 is a module for detecting LOH of one or more HLA-I genes and/or tumor mutational burden and includes the processes that embody the functionality of the present disclosure (e.g., as embodied in the devices as described herein).

FIG. 10 illustrates an example of a computing system in accordance with one embodiment. In System 1000, Device 1100 (e.g., as described above and illustrated in FIG. 11) is connected to Network 1004, which is also connected to Device 1006. In some embodiments, Device 1006 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system. Devices 1100 and 1006 may communicate, e.g., using suitable communication interfaces via Network 1004, such as a Local Area Network (LAN), Virtual Private Network (VPN), or the Internet. In some embodiments, Network 1004 can be, for example, the Internet, an intranet, a virtual private network, a cloud network, a wired network, or a wireless network. Devices 1100 and 1006 may communicate, in part or in whole, via wireless or hardwired communications, such as Ethernet, IEEE 802.11b wireless, or the like. Additionally, Devices 1100 and 1006 may communicate, e.g., using suitable communication interfaces, via a second network, such as a mobile/cellular network. Communication between Devices 1100 and 1006 may further include or communicate with various servers such as a mail server, mobile server, media server, telephone server, and the like. In some embodiments, Devices 1100 and 1006 can communicate directly (instead of, or in addition to, communicating via Network 1004), e.g., via wireless or hardwired communications, such as Ethernet, IEEE 802.11b wireless, or the like. In some embodiments, Devices 1100 and 1006 communicate via Communications 1008, which can be a direct connection or can occur via a network (e.g., Network 1004).

One or all of Devices 1100 and 1006 generally include logic (e.g., http web server logic) or is programmed to format data, accessed from local or remote databases or other sources of data and content, for providing and/or receiving information via Network 1004 according to various examples described herein.

FIG. 8 illustrates an exemplary process 800 for detecting methylation level (e.g., of a cluster of two or more CpG dinucleotides), in accordance with some embodiments of the present disclosure. Process 800 is performed, for example, using one or more electronic devices implementing a software program. In some examples, process 800 is performed using a client-server system, and the blocks of process 800 are divided up in any manner between the server and a client device. In other examples, the blocks of process 800 are divided up between the server and multiple client devices. Thus, while portions of process 800 are described herein as being performed by particular devices of a client-server system, it will be appreciated that process 800 is not so limited. In some embodiments, the executed steps can be executed across many systems, e.g., in a cloud environment. In other examples, process 800 is performed using only a client device or only multiple client devices. In process 800, some blocks are, optionally, combined, the order of some blocks is, optionally, changed, and some blocks are, optionally, omitted. In some examples, additional steps may be performed in combination with the process 800. Accordingly, the operations as illustrated (and described in greater detail below) are exemplary by nature and, as such, should not be viewed as limiting.

At block 802, a plurality of sequence reads of one or more nucleic acids is obtained by sequencing a plurality of nucleic acids or nucleic acid fragments. In some embodiments, the plurality of nucleic acids or nucleic acid fragments corresponds to one or more genomic loci comprising a cluster of two or more CpG dinucleotides. In some embodiments, the sequence reads are obtained using a sequencer, e.g., as described herein or otherwise known in the art. Optionally, prior to obtaining the sequence reads, the plurality of nucleic acids or nucleic acid fragments is isolated from a sample, subjected to cytosine conversion (e.g., by bisulfite treatment, TET-assisted bisulfite treatment, TET-assisted pyridine borane treatment, oxidative bisulfite treatment, or APOBEC treatment), subjected to fragmentation, selectively enriched for genomic loci comprising cluster(s) of CpG dinucleotides, and/or amplified by PCR. At block 804, an exemplary system (e.g., one or more electronic devices) determines a consensus methylation pattern for the cluster, representing each CpG dinucleotide in the cluster for which methylation was detected in at least one sequence read. At block 806, an exemplary system (e.g., one or more electronic devices) generates a CCF for the cluster representing a fraction of sequence reads corresponding to the cluster that show the consensus methylation pattern out of a total number of sequence reads from the plurality corresponding to the cluster. Optionally, prior to determining the consensus methylation pattern and generating the CCF, sequence reads are demultiplexed, aligned to a reference genome, and/or excluded (e.g., sequence reads that failed to undergo cytosine conversion, sequence reads with a base other than cytosine or thymine at a first position of at least one of the CpG dinucleotides, or sequence reads with a base quality below a threshold base quality).

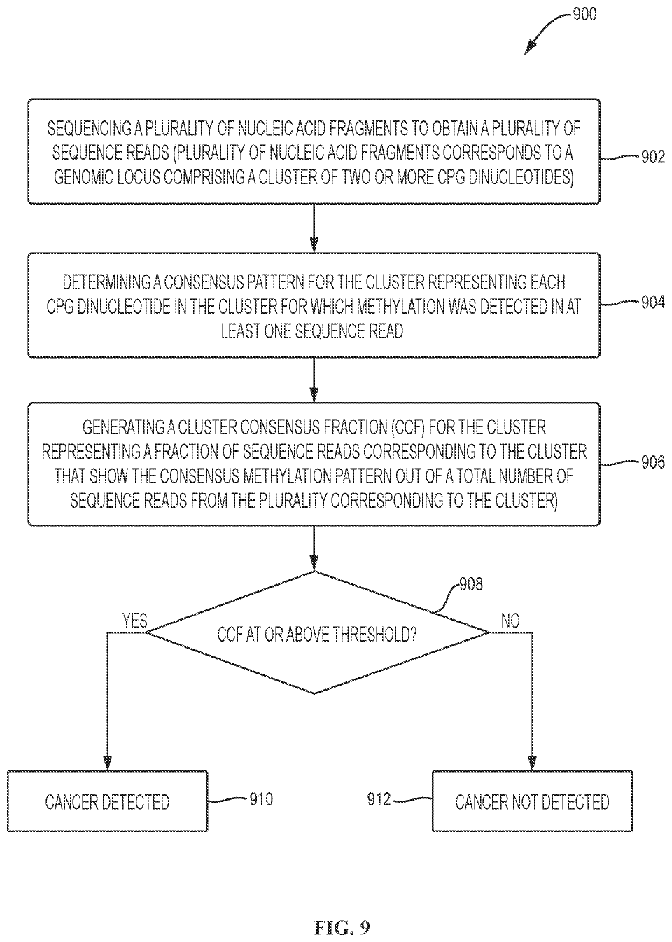

FIG. 9 illustrates an exemplary process 900 for detecting methylation level (e.g., of a cluster of two or more CpG dinucleotides), in accordance with some embodiments of the present disclosure. Process 900 is performed, for example, using one or more electronic devices implementing a software program. In some examples, process 900 is performed using a client-server system, and the blocks of process 900 are divided up in any manner between the server and a client device. In other examples, the blocks of process 900 are divided up between the server and multiple client devices. Thus, while portions of process 900 are described herein as being performed by particular devices of a client-server system, it will be appreciated that process 900 is not so limited. In some embodiments, the executed steps can be executed across many systems, e.g., in a cloud environment. In other examples, process 900 is performed using only a client device or only multiple client devices. In process 900, some blocks are, optionally, combined, the order of some blocks is, optionally, changed, and some blocks are, optionally, omitted. In some examples, additional steps may be performed in combination with the process 900. Accordingly, the operations as illustrated (and described in greater detail below) are exemplary by nature and, as such, should not be viewed as limiting.

At block 902, a plurality of sequence reads of one or more nucleic acids is obtained by sequencing a plurality of nucleic acids or nucleic acid fragments. In some embodiments, the plurality of nucleic acids or nucleic acid fragments corresponds to one or more genomic loci comprising a cluster of two or more CpG dinucleotides. In some embodiments, the sequence reads are obtained using a sequencer, e.g., as described herein or otherwise known in the art. Optionally, prior to obtaining the sequence reads, the plurality of nucleic acids or nucleic acid fragments is isolated from a sample, subjected to cytosine conversion (e.g., by bisulfite treatment, TET-assisted bisulfite treatment, TET-assisted pyridine borane treatment, oxidative bisulfite treatment, or APOBEC treatment), subjected to fragmentation, selectively enriched for genomic loci comprising cluster(s) of CpG dinucleotides, and/or amplified by PCR. At block 904, an exemplary system (e.g., one or more electronic devices) determines a consensus methylation pattern for the cluster, representing each CpG dinucleotide in the cluster for which methylation was detected in at least one sequence read. At block 906, an exemplary system (e.g., one or more electronic devices) generates a CCF for the cluster representing a fraction of sequence reads corresponding to the cluster that show the consensus methylation pattern out of a total number of sequence reads from the plurality corresponding to the cluster. Optionally, prior to determining the consensus methylation pattern and generating the CCF, sequence reads are demultiplexed, aligned to a reference genome, and/or excluded (e.g., sequence reads that failed to undergo cytosine conversion, sequence reads with a base other than cytosine or thymine at a first position of at least one of the CpG dinucleotides, or sequence reads with a base quality below a threshold base quality). At block 908, the CCF is compared to a reference or threshold value. At block 910, if the CCF is at or above the reference or threshold value, cancer or aberrant methylation levels are detected. At block 912, if the CCF is below the reference or threshold value, cancer or aberrant methylation levels is/are not detected, or normal or wild-type methylation levels are detected.

### Reporting

In some embodiments, the methods provided herein comprise generating a report, and/or providing a report to party. In some embodiments, the report comprises one or more treatment options identified for the individual, e.g., based at least in part on methylation levels detected in a sample from the individual as described herein.

In some embodiments, the one or more treatment options are based at least in part on a general amount of methylation detected.

In other embodiments, the one or more treatment options are based at least in part on methylation of one or more specific genomic loci. For example, in some embodiments, the one or more treatment options are based at least in part on methylation of the PITX2 locus or the MGMT locus. In some embodiments, methylation of the PITX2 locus detected in the sample identifies the individual as one who may benefit from treatment comprising anthracycline-based chemotherapy. In some embodiments, methylation of the MGMT locus detected in the sample identifies the individual as one who may benefit from the treatment comprising an alkylating agent.

In some embodiments, the report includes information on the role of methylation (e.g., in general, or in specific genomic loci such as the PITX2 or MGMT loci), in disease, such as in cancer. Such information can include one or more of: information on prognosis of a cancer, information on resistance of the cancer to one or more treatments; information on potential or suggested therapeutic options (e.g., an anti-cancer therapy provided herein, such as anthracycline-based chemotherapy in the case of methylation of the PITX2 locus or an alkylating agent in the case of methylation of the MGMT locus, e.g., according to the methods provided herein); or information on therapeutic options that should be avoided. In some embodiments, the report includes information on the likely effectiveness, acceptability, and/or advisability of applying a therapeutic option (e.g., an anti-cancer therapy provided herein, such as anthracycline-based chemotherapy in the case of methylation of the PITX2 locus or an alkylating agent in the case of methylation of the MGMT locus, e.g., according to the methods provided herein) to an individual having a cancer. In some embodiments, the report includes information or a recommendation on the administration of a treatment (e.g., an anti-cancer therapy provided herein, such as anthracycline-based chemotherapy in the case of methylation of the PITX2 locus or an alkylating agent in the case of methylation of the MGMT locus, e.g., according to the methods provided herein). In some embodiments, the information or recommendation includes the dosage of the treatment and/or a treatment regimen (e.g., as a monotherapy, or in combination with other treatments, such as a second anti-cancer agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: sequencing, by a sequencer, a plurality of nucleic acid fragments to obtain a plurality of sequence reads, wherein the plurality of nucleic acid fragments has undergone cytosine conversion, and wherein the plurality of nucleic acid fragments corresponds to a genomic locus comprising a cluster of two or more CpG dinucleotides; determining, by a processor, a consensus methylation pattern for the cluster, wherein the consensus methylation pattern represents each CpG dinucleotide in the cluster for which methylation was detected in at least one sequence read from the plurality of sequence reads based on the cytosine conversion; generating, by a processor, a cluster consensus fraction (CCF) for the cluster, wherein the CCF represents a fraction of sequence reads corresponding to the cluster that show the consensus methylation pattern out of a total number of sequence reads from the plurality corresponding to the cluster, thereby detecting methylation level of the cluster; and generating a report, e.g., based at least in part on the CCF. In some embodiments, the methods further comprise obtaining a sample, such as a sample described herein, from an individual, e.g., an individual having a cancer; isolating nucleic acids or nucleic acid fragments from the sample; and/or subjected the nucleic acids or nucleic acid fragments to cytosine conversion, e.g., according to any of the methods described herein.

In some embodiments, a report generated according to the methods provided herein comprises one or more of: information about methylation level (e.g., in general, or in specific genomic loci such as the PITX2 or MGMT loci) in the sample; an identifier for the individual from which the sample was obtained; information on the role of methylation in disease (e.g., such as in cancer); information on prognosis, resistance, or potential or suggested therapeutic options (e.g., an anti-cancer therapy provided herein, such as anthracycline-based chemotherapy in the case of methylation of the PITX2 locus or an alkylating agent in the case of methylation of the MGMT locus, e.g., according to the methods provided herein); information on the likely effectiveness, acceptability, or the advisability of applying a therapeutic option (e.g., an anti-cancer therapy provided herein, such as anthracycline-based chemotherapy in the case of methylation of the PITX2 locus or an alkylating agent in the case of methylation of the MGMT locus, e.g., according to the methods provided herein) to the individual; a recommendation or information on the administration of a treatment (e.g., an anti-cancer therapy provided herein, such as anthracycline-based chemotherapy in the case of methylation of the PITX2 locus or an alkylating agent in the case of methylation of the MGMT locus, e.g., according to the methods provided herein); or a recommendation or information on the dosage or treatment regimen of a treatment (e.g., an anti-cancer therapy provided herein, such as anthracycline-based chemotherapy in the case of methylation of the PITX2 locus or an alkylating agent in the case of methylation of the MGMT locus, e.g., according to the methods provided herein), e.g., in combination with other treatments (e.g., a second anti-cancer therapy). In some embodiments, the report generated is a personalized cancer report.

A report according to the present disclosure may be in an electronic, web-based, or paper form. The report may be provided to an individual or a patient (e.g., an individual or a patient with a cancer), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting methylation level in a sample obtained from an individual (e.g., an individual having a cancer).

### Immune Checkpoint Inhibitors and Anti-Cancer Therapies

Certain aspects of the present disclosure relate to immune checkpoint inhibitors (ICIs). As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCN1 (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments according to any of the embodiments described herein, the ICI comprises a PD-1 antagonist/inhibitor or a PD-L1 antagonist/inhibitor.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist/inhibitor is a molecule that inhibits the binding of PD-1 to its ligand binding partners. In a specific embodiment, the PD-1 ligand binding partners are PD-L1 and/or PD-L2. In another instance, a PD-L1 binding antagonist/inhibitor is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is MDX-1 106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IB1308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANBO11), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist/inhibitor of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the immune checkpoint inhibitor is monovalent and/or monospecific. In some embodiments, the immune checkpoint inhibitor is multivalent and/or multispecific.

In some embodiments, the immune checkpoint inhibitor may be administered in combination with an immunoregulatory molecule or a cytokine. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-10, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, 1309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the methods provided herein comprise administering to an individual a treatment that comprises an immune checkpoint inhibitor (e.g., as described supra). In some embodiments, the methods provided herein comprise selecting/identifying a treatment or one or more treatment options for an individual, wherein the treatment or the one or more treatment options comprise an immune checkpoint inhibitor (e.g., as described supra). In some embodiments, the treatment or the one or more treatment options further comprise an additional anti-cancer therapy. In some embodiments, the additional anti-cancer therapy is an agent other than an ICI (e.g., as described infra), or a second ICI (e.g., as described supra).

In some embodiments, the anti-cancer therapy comprises a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, an anti-hormonal agent, a kinase inhibitor, a peptide, a gene therapy, a vaccine, a platinum-based chemotherapeutic agent, an immunotherapy, a growth inhibitory agent, a cytotoxic agent, an antimetabolite chemotherapeutic agent, or any combination thereof.

In some embodiments, the anti-cancer therapy comprises a chemotherapy. In some embodiments, the methods provided herein comprise administering to the individual a chemotherapy, e.g., in combination with another anti-cancer therapy such as an immune checkpoint inhibitor. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammal1 and calicheamicin omegal1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure, such as an immune checkpoint inhibitor, are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor. In some embodiments, the methods provided herein comprise administering to the individual a kinase inhibitor, e.g., in combination with another anti-cancer therapy such as an immune checkpoint inhibitor. Examples of kinase inhibitors include those that target one or more receptor tyrosine kinases, e.g., BCR-ABL, B-Raf, EGFR, HER-2/ErbB2, IGF-IR, PDGFR-a, PDGFR-3, cKit, Flt-4, Flt3, FGFR1, FGFR3, FGFR4, CSF1R, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent. In some embodiments, the methods provided herein comprise administering to the individual an anti-angiogenic agent, e.g., in combination with another anti-cancer therapy such as an immune checkpoint inhibitor. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy. In some embodiments, the methods provided herein comprise administering to the individual an anti-DNA repair therapy, e.g., in combination with another anti-cancer therapy such as an immune checkpoint inhibitor. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer. In some embodiments, the methods provided herein comprise administering to the individual a radiosensitizer, e.g., in combination with another anti-cancer therapy such as an immune checkpoint inhibitor. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent. In some embodiments, the methods provided herein comprise administering to the individual an anti-inflammatory agent, e.g., in combination with another anti-cancer therapy such as an immune checkpoint inhibitor. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the anti-cancer therapy comprises an anti-hormonal agent. In some embodiments, the methods provided herein comprise administering to the individual an anti-hormonal agent, e.g., in combination with another anti-cancer therapy such as an immune checkpoint inhibitor. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent. In some embodiments, the methods provided herein comprise administering to the individual an antimetabolite chemotherapeutic agent, e.g., in combination with another anti-cancer therapy such as an immune checkpoint inhibitor. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent. In some embodiments, the methods provided herein comprise administering to the individual a platinum-based chemotherapeutic agent, e.g., in combination with another anti-cancer therapy such as an immune checkpoint inhibitor. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some embodiments, the anti-cancer therapy comprises a heat shock protein (HSP) inhibitor, a MYC inhibitor, an HDAC inhibitor, an immunotherapy, a neoantigen, a vaccine, or a cellular therapy. In some embodiments, the anti-cancer therapy includes one or more of a chemotherapy, a VEGF inhibitor, an Integrin β3 inhibitor, a statin, an EGFR inhibitor, an mTOR inhibitor, a PI3K inhibitor, a MAPK inhibitor, or a CDK4/6 inhibitor.

In some embodiments, the anti-cancer therapy comprises a kinase inhibitor. In some embodiments, the methods provided herein comprise administering to the individual a kinase inhibitor, e.g., in combination with another anti-cancer therapy such as an immune checkpoint inhibitor. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). Additional examples of ALK kinase inhibitors that may be used according to any of the methods provided herein are described in examples 3-39 of WO2005016894, which is incorporated herein by reference.

In some embodiments, the anti-cancer therapy comprises a heat shock protein (HSP) inhibitor. In some embodiments, the methods provided herein comprise administering to the individual an HSP inhibitor, e.g., in combination with another anti-cancer therapy such as an immune checkpoint inhibitor. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor. In some embodiments, the methods provided herein comprise administering to the individual a MYC inhibitor, e.g., in combination with another anti-cancer therapy such as an immune checkpoint inhibitor. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor. In some embodiments, the methods provided herein comprise administering to the individual an HDAC inhibitor, e.g., in combination with another anti-cancer therapy such as an immune checkpoint inhibitor. In some embodiments, the HDAC inhibitor is belinostat (PXD101, Beleodaq®), SAHA (vorinostat, suberoylanilide hydroxamine, Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, CIA, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor. In some embodiments, the methods provided herein comprise administering to the individual a VEGF inhibitor, e.g., in combination with another anti-cancer therapy such as an immune checkpoint inhibitor. In some embodiments, the VEGF inhibitor is Bevacizumab (Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin β3 inhibitor. In some embodiments, the methods provided herein comprise administering to the individual an integrin β3 inhibitor, e.g., in combination with another anti-cancer therapy such as an immune checkpoint inhibitor. In some embodiments, the integrin β inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNT095) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbp3 integrin inhibitor. In some embodiments, the methods provided herein comprise administering to the individual an αIIbp3 integrin inhibitor, e.g., in combination with another anti-cancer therapy such as an immune checkpoint inhibitor. In some embodiments, the αIIbp3 integrin inhibitor is abciximab, eptifibatide (Integrilin®), or tirofiban (Aggrastat®).

In some embodiments, the anti-cancer therapy comprises a statin or a statin-based agent. In some embodiments, the methods provided herein comprise administering to the individual a statin or a statin-based agent, e.g., in combination with another anti-cancer therapy such as an immune checkpoint inhibitor. In some embodiments, the statin or statin-based agent is simvastatin, atorvastatin, fluvastatin, pitavastatin, pravastatin, rosuvastatin, or cerivastatin.

In some embodiments, the anti-cancer therapy comprises an mTOR inhibitor. In some embodiments, the methods provided herein comprise administering to the individual an mTOR inhibitor, e.g., in combination with another anti-cancer therapy such as an immune checkpoint inhibitor. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a PI3K inhibitor. In some embodiments, the methods provided herein comprise administering to the individual a PI3K inhibitor, e.g., in combination with another anti-cancer therapy such as an immune checkpoint inhibitor. In some embodiments, the PI3K inhibitor is GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDCO0032, IPI145, INK1117 (MLN1117), SAR260301, KIN-193 (AZD6482), duvelisib, GS-9820, GSK2636771, GDC-0980, AMG319, pazobanib, or alpelisib (BYL719, Piqray).

In some embodiments, the anti-cancer therapy comprises a MAPK inhibitor. In some embodiments, the methods provided herein comprise administering to the individual a MAPK inhibitor, e.g., in combination with another anti-cancer therapy such as an immune checkpoint inhibitor. In some embodiments, the MAPK inhibitor is SB203580, SKF-86002, BIRB-796, SC-409, RJW-67657, BIRB-796, VX-745, RO3201195, SB-242235, or MW181.

In some embodiments, the anti-cancer therapy comprises a CDK4/6 inhibitor. In some embodiments, the methods provided herein comprise administering to the individual a CDK4/6 inhibitor, e.g., in combination with another anti-cancer therapy such as an immune checkpoint inhibitor. In some embodiments, the CDK4/6 inhibitor is ribociclib (Kisqali®, LEE011), palbociclib (PD0332991, Ibrance®), or abemaciclib (LY2835219).

In some embodiments, the anti-cancer therapy comprises an EGFR inhibitor. In some embodiments, the methods provided herein comprise administering to the individual an EGFR inhibitor, e.g., in combination with another anti-cancer therapy such as an immune checkpoint inhibitor. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (ACO0010), rociletinib (CO-1686), BMS-690514, YH5448, PF-06747775, ASP8273, PF299804, AP26113, or erlotinib. In some embodiments, the EGFR inhibitor is gefitinib or cetuximab.

In some embodiments, the anti-cancer therapy comprises a cancer immunotherapy, such as a cancer vaccine, cell-based therapy, T cell receptor (TCR)-based therapy, adjuvant immunotherapy, cytokine immunotherapy, and oncolytic virus therapy. In some embodiments, the methods provided herein comprise administering to the individual a cancer immunotherapy, such as a cancer vaccine, cell-based therapy, T cell receptor (TCR)-based therapy, adjuvant immunotherapy, cytokine immunotherapy, and oncolytic virus therapy, e.g., in combination with another anti-cancer therapy such as an immune checkpoint inhibitor. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen expressed by a cancer of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et ah, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21), 2013). In some embodiments, such cancer vaccines augment the anti-cancer response.

In some embodiments, the cancer vaccine comprises a polynucleotide that encodes a neoantigen, e.g., a neoantigen expressed by a cancer of the disclosure. In some embodiments, the cancer vaccine comprises DNA or RNA that encodes a neoantigen. In some embodiments, the cancer vaccine comprises a polynucleotide that encodes a neoantigen. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises a nucleic acid molecule, such as a dsRNA, an siRNA, or an shRNA. In some embodiments, the methods provided herein comprise administering to the individual a nucleic acid molecule, such as a dsRNA, an siRNA, or an shRNA, e.g., in combination with another anti-cancer therapy. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. Methods for designing, optimizing, producing, and using dsRNAs, siRNAs, or shRNAs, are known in the art.

In some aspects, provided herein are therapeutic formulations comprising an anti-cancer therapy provided herein (e.g., an immune checkpoint inhibitor and/or an additional anti-cancer therapy), and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 1 19, Marcel Dekker, 2002.

Formulations to be used for in vivo administration are sterile. This is readily accomplished by filtration through sterile filtration membranes or other methods known in the art.

In some embodiments, an immune checkpoint inhibitor is administered as a monotherapy.

In some embodiments, the immune checkpoint inhibitor is a first line immune checkpoint inhibitor. In some embodiments, the immune checkpoint inhibitor is a second line immune checkpoint inhibitor. In some embodiments, an immune checkpoint inhibitor is administered in combination with one or more additional anti-cancer therapies or treatments. In some embodiments, the one or more additional anti-cancer therapies or treatments include one or more anti-cancer therapies described herein. In some embodiments, the methods of the present disclosure comprise administration of any combination of any of the immune checkpoint inhibitors and anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an immune checkpoint inhibitor may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an immune checkpoint inhibitor may be administered in conjunction with a radiation therapy.

### IV. Exemplary Embodiments

The following exemplary embodiments are representative of some aspects of the invention:
Embodiment 1. A method of detecting one or more of a methylation level or an unmethylation level of a cluster of two or more CpG dinucleotides in a sample from a subject, comprising:
   obtaining a plurality of nucleic acid fragments from the sample;
   amplifying the plurality of nucleic acid fragments;
   sequencing, by a sequencer, the plurality of amplified nucleic acid fragments to obtain a plurality of sequence reads, wherein at least the plurality of amplified nucleic acid fragments has undergone cytosine conversion, and wherein the plurality of nucleic acid fragments corresponds to a genomic locus comprising a cluster of two or more CpG dinucleotides;
   determining, by a processor, a consensus methylation pattern for the cluster, wherein the consensus methylation pattern represents each CpG dinucleotide in the cluster for which methylation was detected based on the cytosine conversion in at least one sequence read from the plurality of sequence reads;
   generating, by a processor, a cluster consensus fraction (CCF) for the cluster, wherein the CCF represents a fraction of sequence reads corresponding to the cluster that show the consensus methylation pattern out of a total number of sequence reads from the plurality corresponding to the cluster;
   detecting one or more of the methylation level or the unmethylation level of the cluster based on the CCF; and
   generating a genomic profile for the subject based on the detected methylation level, the detected unmethylation level, or both.
Embodiment 2. The method of embodiment 1, wherein the CCF is at or above a threshold or reference value, and the method further comprises: detecting presence of cancer nucleic acids in the plurality of nucleic acid fragments, based at least in part on the CCF being at or above the threshold or reference value.
Embodiment 3. The method of embodiment 1, wherein the CCF is below a threshold or reference value, and the method further comprises: detecting absence of cancer nucleic acids in the plurality of nucleic acid fragments, based at least in part on the CCF being below the threshold or reference value.
Embodiment 4. The method of any one of embodiments 1-3, comprising determining a consensus methylation pattern and CCF for more than one cluster.
Embodiment 5. The method of embodiment 4, wherein the more than one cluster corresponds to more than one genomic locus.
Embodiment 6. The method of embodiment 4 or embodiment 5, comprising determining a consensus methylation pattern and CCF for more than 1,000 clusters.
Embodiment 7. The method of embodiment 4 or embodiment 5, comprising determining a consensus methylation pattern and CCF for between 10 and 100,000 clusters.
Embodiment 8. The method of any one of embodiments 1-7, comprising determining a consensus methylation pattern and CCF for up to 1 million clusters.
Embodiment 9. The method of any one of embodiments 1-8, wherein the plurality of sequence reads comprises at least 100 sequence reads corresponding to the cluster.
Embodiment 10. The method of embodiment 9, wherein the plurality of sequence reads comprises at least 1000 sequence reads corresponding to the cluster.
Embodiment 11. The method of any one of embodiments 1-8, wherein the plurality of sequence reads comprises between 1 and 5 sequence reads corresponding to the cluster.
Embodiment 12. The method of any one of embodiments 1-11, wherein at least one CpG dinucleotide in the cluster is unmethylated in the consensus methylation pattern.
Embodiment 13. The method of any one of embodiments 1-12, wherein at least one CpG dinucleotide in the cluster is methylated in the consensus methylation pattern.
Embodiment 14. The method of any one of embodiments 1-13, wherein at least one cluster comprises two or more CpG dinucleotides.
Embodiment 15. The method of embodiment 14, wherein each cluster comprises two or more CpG dinucleotides.
Embodiment 16. The method of any one of embodiments 1-13, wherein at least one cluster comprises five or more CpG dinucleotides.
Embodiment 17. The method of embodiment 16, wherein each cluster comprises five or more CpG dinucleotides.
Embodiment 18. The method of any one of embodiments 1-17, wherein at least one cluster comprises six or more CpG dinucleotides.
Embodiment 19. The method of any one of embodiments 1-18, wherein all sites in the cluster except one are unmethylated in the consensus methylation pattern.
Embodiment 20. The method of any one of embodiments 1-18, wherein all sites in the cluster except two are unmethylated in the consensus methylation pattern.
Embodiment 21. The method of any one of embodiments 1-18, wherein at most 1 site in the cluster is methylated in the consensus methylation pattern.
Embodiment 22. The method of any one of embodiments 1-18, wherein at most 2 sites in the cluster are methylated in the consensus methylation pattern.
Embodiment 23. The method of any one of embodiments 1-18, wherein at most 10% of sites in the cluster are methylated in the consensus methylation pattern.
Embodiment 24. The method of any one of embodiments 1-18, wherein at most 25% of sites in the cluster are methylated in the consensus methylation pattern.
Embodiment 25. The method of any one of embodiments 1-20, wherein greater than 75% of sites in the cluster are methylated in the consensus methylation pattern.
Embodiment 26. The method of any one of embodiments 1-20, wherein greater than 50% of sites in the cluster are methylated in the consensus methylation pattern.
Embodiment 27. The method of any one of embodiments 1-20, wherein greater than 25% of sites in the cluster are methylated in the consensus methylation pattern.
Embodiment 28. The method of any one of embodiments 1-27, wherein the plurality of sequence reads is obtained from whole-genome methyl sequencing (WGMS) or next-generation sequencing (NGS).
Embodiment 29. The method of any one of embodiments 1-28, wherein the plurality of sequence reads includes paired-end sequence reads.
Embodiment 30. The method of embodiment 29, wherein the consensus methylation pattern and CCF are determined based on paired-end sequence reads corresponding to the cluster.
Embodiment 31. The method of any one of embodiments 1-28, wherein the plurality of sequence reads includes unpaired sequence reads.
Embodiment 32. The method of any one of embodiments 1-31, further comprising, prior to determining the consensus methylation pattern and CCF, demultiplexing sequence reads from the plurality of sequence reads.
Embodiment 33. The method of any one of embodiments 1-32, further comprising, prior to determining the consensus methylation pattern and CCF, performing three-letter alignment of sequence reads from the plurality to a reference genome.
Embodiment 34. The method of any one of embodiments 1-33, further comprising, prior to determining the consensus methylation pattern and CCF, excluding sequencing reads from the plurality that failed to undergo cytosine conversion.
Embodiment 35. The method of any one of embodiments 1-34, further comprising, prior to determining the consensus methylation pattern and CCF, excluding sequence reads with a base other than cytosine or thymine at a first position of at least one of the CpG dinucleotides.
Embodiment 36. The method of any one of embodiments 1-35, further comprising, prior to determining the consensus methylation pattern and CCF, excluding sequence reads with a base quality below a threshold base quality.
Embodiment 37. The method of any one of embodiments 1-36, wherein the consensus methylation pattern and CCF are determined based on sequence reads that cover a plurality of CpG dinucleotides in the cluster.
Embodiment 38. The method of embodiment 37, wherein the consensus methylation pattern and CCF are determined based on sequence reads that cover at least 50% of CpG dinucleotides in the cluster.
Embodiment 39. The method of embodiment 37, wherein the consensus methylation pattern and CCF are determined based on sequence reads that cover at least 90% of CpG dinucleotides in the cluster.
Embodiment 40. The method of embodiment 37, wherein the consensus methylation pattern and CCF are determined based on sequence reads that cover all CpG dinucleotides in the cluster.
Embodiment 41. The method of any one of embodiments 1-40, wherein the plurality of nucleic acid fragments has undergone cytosine conversion by bisulfite treatment.
Embodiment 42. The method of any one of embodiments 1-40, wherein the plurality of nucleic acid fragments has undergone cytosine conversion by TET-assisted bisulfite treatment, TET-assisted pyridine borane treatment, oxidative bisulfite treatment, or APOBEC treatment.
Embodiment 43. The method of any one of embodiments 1-40, further comprising, prior to providing the plurality of sequence reads, treating a plurality of nucleic acids or nucleic acid fragments with bisulfite.
Embodiment 44. The method of any one of embodiments 1-40, further comprising, prior to providing the plurality of sequence reads, treating a plurality of nucleic acids or nucleic acid fragments with TET-assisted bisulfite treatment, TET-assisted pyridine borane treatment, oxidative bisulfite treatment, or APOBEC treatment.
Embodiment 45. The method of any one of embodiments 1-44, further comprising, prior to providing the plurality of sequence reads, subjecting a plurality of nucleic acids to fragmentation.
Embodiment 46. The method of any one of embodiments 1-45, further comprising, prior to providing the plurality of sequence reads, selectively enriching for a plurality of nucleic acids or nucleic acid fragments corresponding to a genomic locus that comprises a cluster of two or more CpG dinucleotides to produce an enriched sample.
Embodiment 47. The method of any one of embodiments 1-46, wherein the amplification of the plurality of nucleic acids or nucleic acid fragments is performed by polymerase chain reaction (PCR).
Embodiment 48. The method of any one of embodiments 1-47, further comprising, prior to providing the plurality of sequence reads, isolating the plurality of nucleic acids from the sample.
Embodiment 49. The method of embodiment 48, wherein the sample comprises tumor cells and/or tumor nucleic acids.
Embodiment 50. The method of embodiment 49, wherein the sample further comprises non-tumor cells and/or non-tumor nucleic acids.
Embodiment 51. The method of embodiment 50, wherein the sample comprises a fraction of tumor nucleic acids that is less than 1% of total nucleic acids.
Embodiment 52. The method of embodiment 50, wherein the sample comprises a fraction of tumor nucleic acids that is less than 0.1% of total nucleic acids.
Embodiment 53. The method of any one of embodiments 50-52, wherein the sample comprises a fraction of tumor nucleic acids that is at least 0.01% of total nucleic acids.
Embodiment 54. The method of any one of embodiments 48-53, wherein the sample comprises tumor cell-free DNA (cfDNA), circulating cell-free DNA (ccfDNA), or circulating tumor DNA (ctDNA).
Embodiment 55. The method of any one of embodiments 48-53, wherein the sample comprises fluid, cells, or tissue.
Embodiment 56. The method of embodiment 55, wherein the sample comprises blood or plasma.
Embodiment 57. The method of any one of embodiments 48-53, wherein the sample comprises a tumor biopsy or a circulating tumor cell.
Embodiment 58. The method of any one of embodiments 1-57, wherein the sample is a tissue sample, and the method further comprises: subjecting a plurality of nucleic acid molecules in the tissue to fragmentation to create the plurality of nucleic acid fragments.
Embodiment 59. The method of embodiment 58, further comprising: ligating one or more adapters onto one or more nucleic acid fragments from the plurality of nucleic acid fragments prior to amplifying the plurality of nucleic acid fragments.
Embodiment 60. A method of detecting cancer in an individual, comprising detecting the methylation level or the unmethylation level according to the method of any one of embodiments 1-59 in a sample comprising a plurality of nucleic acids obtained from the individual, wherein the methylation level or the unmethylation level detected in the sample identifies the individual as having cancer.
Embodiment 61. A method of screening an individual suspected of having cancer, comprising detecting the methylation level or the unmethylation level according to the method of any one of embodiments 1-59 in a sample comprising a plurality of nucleic acids obtained from the individual, wherein the methylation level or the unmethylation level detected in the sample identifies the individual as likely to have cancer.
Embodiment 62. A method of determining prognosis of an individual having cancer, comprising detecting the methylation level or the unmethylation level according to the method of any one of embodiments 1-59 in a sample comprising a plurality of nucleic acids obtained from the individual, wherein the methylation level or the unmethylation level detected in the sample determines at least in part the prognosis of the individual.
Embodiment 63. A method of predicting survival of an individual having cancer, comprising detecting the methylation level or the unmethylation level according to the method of any one of embodiments 1-59 in a sample comprising a plurality of nucleic acids obtained from the individual, wherein the methylation level or the unmethylation level detected in the sample predicts at least in part the survival of the individual.
Embodiment 64. The method of embodiment 63, wherein the methylation level detected in the sample is higher than a threshold or reference value, and wherein survival of the individual is predicted to be decreased, as compared to survival of an individual whose sample has a methylation level lower than the threshold or reference value.
Embodiment 65. A method of predicting tumor burden of an individual having cancer, comprising detecting the methylation level or the unmethylation level according to the method of any one of embodiments 1-59 in a sample comprising a plurality of nucleic acids obtained from the individual, wherein the methylation level or the unmethylation level detected in the sample predicts at least in part the tumor burden of the individual.
Embodiment 66. The method of embodiment 65, wherein the methylation level detected in the sample is higher than a threshold or reference value, and wherein tumor burden of the individual is predicted to be increased, as compared to tumor burden of an individual whose sample has a methylation level lower than the threshold or reference value.
Embodiment 67. A method of predicting responsiveness to treatment of an individual having cancer, comprising detecting the methylation level or the unmethylation level according to the method of any one of embodiments 1-59 in a sample comprising a plurality of nucleic acids obtained from the individual, wherein the methylation level or the unmethylation level detected in the sample is used at least in part to predict responsiveness of the individual to a treatment.
Embodiment 68. A method of identifying an individual having cancer who may benefit from a treatment comprising anthracycline-based chemotherapy, the method comprising detecting the methylation level or the unmethylation level according to the method of any one of embodiments 1-59 in a sample comprising a plurality of nucleic acids obtained from the individual, wherein the plurality of nucleic acids includes one or more nucleic acids corresponding to a PITX2 locus, wherein methylation of the PITX2 locus detected in the sample identifies the individual as one who may benefit from the treatment comprising anthracycline-based chemotherapy.
Embodiment 69. A method of selecting a therapy for an individual having cancer, the method comprising detecting the methylation level or the unmethylation level according to the method of any one of embodiments 1-59 in a sample comprising a plurality of nucleic acids obtained from the individual, wherein the plurality of nucleic acids includes one or more nucleic acids corresponding to a PITX2 locus, wherein methylation of the PITX2 locus detected in the sample identifies the individual as one who may benefit from treatment comprising anthracycline-based chemotherapy.
Embodiment 70. A method of identifying one or more treatment options for an individual having cancer, the method comprising:
   (a) detecting the methylation level or the unmethylation level according to the method of any one of embodiments 1-59 in a sample comprising a plurality of nucleic acids obtained from the individual, wherein the plurality of nucleic acids includes one or more nucleic acids corresponding to a PITX2 locus; and
   (b) generating a report comprising one or more treatment options identified for the individual based at least in part on methylation of the PITX2 locus detected in the sample, wherein the one or more treatment options comprise anthracycline-based chemotherapy.
Embodiment 71. A method of treating or delaying progression of cancer, comprising:
   (a) detecting the methylation level or the unmethylation level according to the method of any one of embodiments 1-59 in a sample comprising a plurality of nucleic acids obtained from the individual, wherein the plurality of nucleic acids includes one or more nucleic acids corresponding to a PITX2 locus; and
   (b) administering to the individual an effective amount of anthracycline-based chemotherapy.
Embodiment 72. A method of identifying an individual having cancer who may benefit from a treatment comprising an alkylating agent, the method comprising detecting the methylation level or the unmethylation level according to the method of any one of embodiments 1-59 in a sample comprising a plurality of nucleic acids obtained from the individual, wherein the plurality of nucleic acids includes one or more nucleic acids corresponding to an MGMT locus, wherein methylation of the MGMT locus detected in the sample identifies the individual as one who may benefit from the treatment comprising an alkylating agent.
Embodiment 73. A method of selecting a therapy for an individual having cancer, the method comprising detecting the methylation level or the unmethylation level according to the method of any one of embodiments 1-59 in a sample comprising a plurality of nucleic acids obtained from the individual, wherein the plurality of nucleic acids includes one or more nucleic acids corresponding to an MGMT locus, wherein methylation of the MGMT locus detected in the sample identifies the individual as one who may benefit from treatment comprising an alkylating agent.
Embodiment 74. A method of identifying one or more treatment options for an individual having cancer, the method comprising:
   (a) detecting the methylation level or the unmethylation level according to the method of any one of embodiments 1-59 in a sample comprising a plurality of nucleic acids obtained from the individual, wherein the plurality of nucleic acids includes one or more nucleic acids corresponding to an MGMT locus; and
   (b) generating a report comprising one or more treatment options identified for the individual based at least in part on methylation of the MGMT locus detected in the sample, wherein the one or more treatment options comprise an alkylating agent.
Embodiment 75. A method of treating or delaying progression of cancer, comprising:
   (a) detecting the methylation level or the unmethylation level according to the method of any one of embodiments 1-59 in a sample comprising a plurality of nucleic acids obtained from the individual, wherein the plurality of nucleic acids includes one or more nucleic acids corresponding to an MGMT locus; and
   (b) administering to the individual an effective amount of an alkylating agent.
Embodiment 76. A method of monitoring response of an individual being treated for cancer, comprising:
   (a) administering a treatment to an individual having cancer; and
   (b) detecting the methylation level or the unmethylation level according to the method of any one of embodiments 1-59 in a sample comprising a plurality of nucleic acids obtained from the individual after treatment, wherein the methylation level or the unmethylation level detected in the sample is used at least in part to monitor response to the treatment.
Embodiment 77. The method of embodiment 76, wherein detection of a methylation level after treatment that is less than a methylation level prior to treatment, or less than a threshold or reference value, indicates that the individual has responded to treatment.
Embodiment 78. The method of embodiment 76, wherein detection of a methylation level after treatment that is not greater than a methylation level prior to treatment, or less than a threshold or reference value, indicates that the individual has responded to treatment.
Embodiment 79. A method of monitoring a cancer in an individual, comprising:
   (a) detecting the methylation level or the unmethylation level according to the method of any one of embodiments 1-59 in a first sample comprising a plurality of nucleic acids obtained from the individual;
   (b) detecting the methylation level or the unmethylation level according to the method of any one of embodiments 1-57 in a second sample comprising a plurality of nucleic acids obtained from the individual, wherein the second sample is obtained from the individual after the first sample; and
   (c) determining a difference in methylation level between the first and second samples, thereby monitoring the cancer in the individual.
Embodiment 80. A method of monitoring response of an individual being treated for cancer, comprising:
   (a) detecting the methylation level or the unmethylation level according to the method of any one of embodiments 1-59 in a first sample comprising a plurality of nucleic acids obtained from the individual;
   (b) after the first sample is obtained from the individual, administering a treatment to the individual;
   (c) detecting the methylation level or the unmethylation level according to the method of any one of embodiments 1-59 in a second sample comprising a plurality of nucleic acids obtained from the individual, wherein the second sample is obtained from the individual after administration of the treatment; and
   (d) determining a difference in methylation level between the first and second samples, thereby monitoring response of the individual to the treatment.
Embodiment 81. A method of detecting one or more of a methylation level or an unmethylation level of a cluster of two or more CpG dinucleotides from a sample, comprising:
   obtaining a plurality of sequence reads from a plurality of nucleic acid fragments exhibiting cytosine conversion;
   determining, by a processor, a consensus methylation pattern for a cluster of two or more CpG dinucleotides at a locus, wherein the consensus methylation pattern represents each CpG dinucleotide in the cluster for which methylation was detected;
   generating, by a processor, a cluster consensus fraction (CCF) for the cluster, wherein the CCF represents a fraction of sequence reads corresponding to the cluster that show the consensus methylation pattern out of a total number of sequence reads from the plurality corresponding to the cluster; and
   detecting, by the processor, one or more of the methylation level or the unmethylation level of the cluster based on the CCF.
Embodiment 82. A method of detecting one or more of a methylation level or an unmethylation level of a cluster of two or more CpG dinucleotides, comprising:
   sequencing, by a sequencer, the plurality of nucleic acid fragments to obtain the plurality of sequence reads;
   determining, by a processor, a consensus methylation pattern for the cluster, wherein the consensus methylation pattern represents each CpG dinucleotide in the cluster for which methylation was detected;
   generating, by a processor, a cluster consensus fraction (CCF) for the cluster, wherein the CCF represents a fraction of sequence reads corresponding to the cluster that show the consensus methylation pattern out of a total number of sequence reads from the plurality corresponding to the cluster, thereby detecting one or more of the methylation level or the unmethylation level of the cluster; and
   detecting, by the processor, one or more of the methylation level or the unmethylation level of the cluster based on the CCF.
Embodiment 83. The method of embodiment 81 or embodiment 82, wherein the consensus methylation pattern represents each CpG dinucleotide in the cluster for which methylation was detected based on the cytosine conversion in at least one sequence read from the plurality.
Embodiment 84. The method of any one of embodiments 81-83, wherein the CCF is at or above a threshold or reference value, and the method further comprises: detecting presence of cancer nucleic acids in the plurality of nucleic acid fragments, based at least in part on the CCF being at or above the threshold or reference value.
Embodiment 85. The method of any one of embodiments 81-83, wherein the CCF is below a threshold or reference value, and the method further comprises: detecting absence of cancer nucleic acids in the plurality of nucleic acid fragments, based at least in part on the CCF being below the threshold or reference value.
Embodiment 86. The method of any one of embodiments 81-85, comprising determining a consensus methylation pattern and CCF for more than one cluster.
Embodiment 87. The method of embodiment 86, wherein the more than one cluster corresponds to more than one genomic locus.
Embodiment 88. The method of embodiment 86 or embodiment 87, comprising determining a consensus methylation pattern and CCF for more than 1,000 clusters.
Embodiment 89. The method of embodiment 86 or embodiment 87, comprising determining a consensus methylation pattern and CCF for between 10 and 100,000 clusters.
Embodiment 90. The method of any one of embodiments 81-89, comprising determining a consensus methylation pattern and CCF for up to 1 million clusters.
Embodiment 91. The method of any one of embodiments 81-90, wherein the plurality of sequence reads comprises at least 100 sequence reads corresponding to the cluster.
Embodiment 92. The method of embodiment 91, wherein the plurality of sequence reads comprises at least 1000 sequence reads corresponding to the cluster.
Embodiment 93. The method of any one of embodiments 81-90, wherein the plurality of sequence reads comprises between 1 and 5 sequence reads corresponding to the cluster.
Embodiment 94. The method of any one of embodiments 81-93, wherein at least one CpG dinucleotide in the cluster is unmethylated in the consensus methylation pattern.
Embodiment 95. The method of any one of embodiments 81-94, wherein at least one CpG dinucleotide in the cluster is methylated in the consensus methylation pattern.
Embodiment 96. The method of any one of embodiments 81-95, wherein at least one cluster comprises two or more CpG dinucleotides.
Embodiment 97. The method of embodiment 96, wherein each cluster comprises two or more CpG dinucleotides.
Embodiment 98. The method of any one of embodiments 81-95, wherein at least one cluster comprises five or more CpG dinucleotides.
Embodiment 99. The method of embodiment 98, wherein each cluster comprises five or more CpG dinucleotides.
Embodiment 100. The method of any one of embodiments 81-99, wherein at least one cluster comprises six or more CpG dinucleotides.
Embodiment 101. The method of any one of embodiments 81-100, wherein all sites in the cluster except one are unmethylated in the consensus methylation pattern.
Embodiment 102. The method of any one of embodiments 81-100, wherein all sites in the cluster except two are unmethylated in the consensus methylation pattern.
Embodiment 103. The method of any one of embodiments 81-100, wherein at most 1 site in the cluster is methylated in the consensus methylation pattern.
Embodiment 104. The method of any one of embodiments 81-100, wherein at most 2 sites in the cluster are methylated in the consensus methylation pattern.
Embodiment 105. The method of any one of embodiments 81-100, wherein at most 10% of sites in the cluster are methylated in the consensus methylation pattern.
Embodiment 106. The method of any one of embodiments 81-100, wherein at most 25% of sites in the cluster are methylated in the consensus methylation pattern.
Embodiment 107. The method of any one of embodiments 81-102, wherein greater than 75% of sites in the cluster are methylated in the consensus methylation pattern.
Embodiment 108. The method of any one of embodiments 81-102, wherein greater than 50% of sites in the cluster are methylated in the consensus methylation pattern.
Embodiment 109. The method of any one of embodiments 81-102, wherein greater than 25% of sites in the cluster are methylated in the consensus methylation pattern.
Embodiment 110. The method of any one of embodiments 81-109, wherein the plurality of sequence reads is obtained from whole-genome methyl sequencing (WGMS) or next-generation sequencing (NGS).
Embodiment 111. The method of any one of embodiments 81-110, wherein the plurality of sequence reads includes paired-end sequence reads.
Embodiment 112. The method of embodiment 111, wherein the consensus methylation pattern and CCF are determined based on paired-end sequence reads corresponding to the cluster.
Embodiment 113. The method of any one of embodiments 81-110, wherein the plurality of sequence reads includes unpaired sequence reads.
Embodiment 114. The method of any one of embodiments 81-113, further comprising, prior to determining the consensus methylation pattern and CCF, demultiplexing sequence reads from the plurality of sequence reads.
Embodiment 115. The method of any one of embodiments 81-114, further comprising, prior to determining the consensus methylation pattern and CCF, performing three-letter alignment of sequence reads from the plurality to a reference genome.
Embodiment 116. The method of any one of embodiments 81-115, further comprising, prior to determining the consensus methylation pattern and CCF, excluding sequencing reads from the plurality that failed to undergo cytosine conversion.
Embodiment 117. The method of any one of embodiments 81-116, further comprising, prior to determining the consensus methylation pattern and CCF, excluding sequence reads with a base other than cytosine or thymine at a first position of at least one of the CpG dinucleotides.
Embodiment 118. The method of any one of embodiments 81-117, further comprising, prior to determining the consensus methylation pattern and CCF, excluding sequence reads with a base quality below a threshold base quality.
Embodiment 119. The method of any one of embodiments 81-118, wherein the consensus methylation pattern and CCF are determined based on sequence reads that cover a plurality of CpG dinucleotides in the cluster.
Embodiment 120. The method of embodiment 119, wherein the consensus methylation pattern and CCF are determined based on sequence reads that cover at least 50% of CpG dinucleotides in the cluster.
Embodiment 121. The method of embodiment 119, wherein the consensus methylation pattern and CCF are determined based on sequence reads that cover at least 90% of CpG dinucleotides in the cluster
Embodiment 122. The method of embodiment 119, wherein the consensus methylation pattern and CCF are determined based on sequence reads that cover all CpG dinucleotides in the cluster.
Embodiment 123. The method of any one of embodiments 81-122, wherein the plurality of nucleic acid fragments has undergone cytosine conversion by bisulfite treatment.
Embodiment 124. The method of any one of embodiments 81-122, wherein the plurality of nucleic acid fragments has undergone cytosine conversion by TET-assisted bisulfite treatment, TET-assisted pyridine borane treatment, oxidative bisulfite treatment, or APOBEC treatment.
Embodiment 125. The method of any one of embodiments 81-122, further comprising, prior to obtaining the plurality of sequence reads, treating a plurality of nucleic acids or nucleic acid fragments with bisulfite.
Embodiment 126. The method of any one of embodiments 81-122, further comprising, prior to obtaining the plurality of sequence reads, treating a plurality of nucleic acids or nucleic acid fragments with TET-assisted bisulfite treatment, TET-assisted pyridine borane treatment, oxidative bisulfite treatment, or APOBEC treatment.
Embodiment 127. The method of any one of embodiments 81-126, further comprising, prior to obtaining the plurality of sequence reads, subjecting a plurality of nucleic acids to fragmentation.
Embodiment 128. The method of any one of embodiments 81-127, further comprising, prior to obtaining the plurality of sequence reads, selectively enriching for a plurality of nucleic acids or nucleic acid fragments corresponding to a genomic locus that comprises a cluster of two or more CpG dinucleotides to produce an enriched sample.
Embodiment 129. The method of any one of embodiments 81-128, wherein the amplification of the plurality of nucleic acids or nucleic acid fragments is performed by polymerase chain reaction (PCR).
Embodiment 130. The method of any one of embodiments 81-129, further comprising, prior to obtaining the plurality of sequence reads, isolating the plurality of nucleic acids from a sample.
Embodiment 131. The method of embodiment 130, wherein the sample comprises tumor cells and/or tumor nucleic acids.
Embodiment 132. The method of embodiment 131, wherein the sample further comprises non-tumor cells and/or non-tumor nucleic acids.
Embodiment 133. The method of embodiment 132, wherein the sample comprises a fraction of tumor nucleic acids that is less than 1% of total nucleic acids.
Embodiment 134. The method of embodiment 132, wherein the sample comprises a fraction of tumor nucleic acids that is less than 0.1% of total nucleic acids.
Embodiment 135. The method of any one of embodiments 132-134, wherein the sample comprises a fraction of tumor nucleic acids that is at least 0.01% of total nucleic acids.
Embodiment 136. The method of any one of embodiments 130-135, wherein the sample comprises tumor cell-free DNA (cfDNA), circulating cell-free DNA (ccfDNA), or circulating tumor DNA (ctDNA).
Embodiment 137. The method of any one of embodiments 130-135, wherein the sample comprises fluid, cells, or tissue.
Embodiment 138. The method of embodiment 137, wherein the sample comprises blood or plasma.
Embodiment 139. The method of any one of embodiments 130-135, wherein the sample comprises a tumor biopsy or a circulating tumor cell.
Embodiment 140. The method of any one of embodiments 81-139, wherein the sample is a tissue sample, and the method further comprises: subjecting a plurality of nucleic acid molecules in the tissue to fragmentation to create the plurality of nucleic acid fragments.
Embodiment 141. The method of embodiment 140, further comprising: ligating one or more adapters onto one or more nucleic acid fragments from the plurality of nucleic acid fragments prior to amplifying the plurality of nucleic acid fragments.
Embodiment 142. A system, comprising:
   one or more processors; and
   a memory configured to store one or more computer program instructions, wherein the one or more computer program instructions when executed by the one or more processors are configured to:
      determine, using the one or more processors, a consensus methylation pattern for a cluster of two or more CpG dinucleotides at a genomic locus, wherein the consensus methylation pattern represents each CpG dinucleotide in the cluster for which methylation was detected in at least one sequence read from a plurality of sequence reads obtained from a plurality of nucleic acid fragments that has undergone cytosine conversion; and
      generate, using the one or more processors, a cluster consensus fraction (CCF) for the cluster, wherein the CCF represents a fraction of sequence reads corresponding to the cluster that show the consensus methylation pattern out of a total number of sequence reads from the plurality corresponding to the cluster.
Embodiment 143. The system of embodiment 142, wherein the CCF is at or above a threshold or reference value, and wherein the one or more computer program instructions when executed by the one or more processors are further configured to: detect, using the one or more processors, presence of cancer nucleic acids in the plurality of nucleic acid fragments, based at least in part on the CCF being at or above the threshold or reference value.
Embodiment 144. The system of embodiment 142, wherein the CCF is below a threshold or reference value, and wherein the one or more computer program instructions when executed by the one or more processors are further configured to: detect, using the one or more processors, absence of cancer nucleic acids in the plurality of nucleic acid fragments, based at least in part on the CCF being below the threshold or reference value.
Embodiment 145. The system of any one of embodiments 142-144, wherein the one or more computer program instructions when executed by the one or more processors are further configured to:
   determine, using the one or more processors, a consensus methylation pattern for more than one cluster of two or more CpG dinucleotides; and
   generate, using the one or more processors, a cluster consensus fraction (CCF) for more than one cluster.
Embodiment 146. The system of embodiment 145, wherein the more than one cluster corresponds to more than one genomic locus.
Embodiment 147. The system of embodiment 145 or embodiment 146, wherein the one or more computer program instructions when executed by the one or more processors are configured to determine a consensus methylation pattern and generate a CCF for more than 1,000 clusters.
Embodiment 148. The system of embodiment 145 or embodiment 146, wherein the one or more computer program instructions when executed by the one or more processors are configured to determine a consensus methylation pattern and generate a CCF for between 10 and 100,000 clusters.
Embodiment 149. The system of embodiment 145 or embodiment 146, wherein the one or more computer program instructions when executed by the one or more processors are configured to determine a consensus methylation pattern and generate a CCF for up to 1 million clusters.
Embodiment 150. The system of any one of embodiments 142-149, wherein the plurality of sequence reads comprises at least 100 sequence reads corresponding to the cluster.
Embodiment 151. The system of embodiment 150, wherein the plurality of sequence reads comprises at least 1000 sequence reads corresponding to the cluster.
Embodiment 152. The system of any one of embodiments 142-149, wherein the plurality of sequence reads comprises between 1 and 5 sequence reads corresponding to the cluster.
Embodiment 153. The system of any one of embodiments 142-152, wherein at least one CpG dinucleotide in the cluster is unmethylated in the consensus methylation pattern.
Embodiment 154. The system of any one of embodiments 142-153, wherein at least one CpG dinucleotide in the cluster is methylated in the consensus methylation pattern.
Embodiment 155. The system of any one of embodiments 142-154, wherein at least one cluster comprises two or more CpG dinucleotides.
Embodiment 156. The system of embodiment 155, wherein each cluster comprises two or more CpG dinucleotides.
Embodiment 157. The system of any one of embodiments 142-154, wherein at least one cluster comprises five or more CpG dinucleotides.
Embodiment 158. The system of embodiment 157, wherein each cluster comprises five or more CpG dinucleotides.
Embodiment 159. The system of any one of embodiments 142-158, wherein at least one cluster comprises six or more CpG dinucleotides.
Embodiment 160. The system of any one of embodiments 142-159, wherein all sites in the cluster except one are unmethylated in the consensus methylation pattern.
Embodiment 161. The system of any one of embodiments 142-159, wherein all sites in the cluster except two are unmethylated in the consensus methylation pattern.
Embodiment 162. The system of any one of embodiments 142-159, wherein at most 1 site in the cluster is methylated in the consensus methylation pattern.
Embodiment 163. The system of any one of embodiments 142-159, wherein at most 2 sites in the cluster are methylated in the consensus methylation pattern.
Embodiment 164. The system of any one of embodiments 142-159, wherein at most 10% of sites in the cluster are methylated in the consensus methylation pattern.
Embodiment 165. The system of any one of embodiments 142-159, wherein at most 25% of sites in the cluster are methylated in the consensus methylation pattern.
Embodiment 166. The system of any one of embodiments 142-161, wherein greater than 75% of sites in the cluster are methylated in the consensus methylation pattern.
Embodiment 167. The system of any one of embodiments 142-161, wherein greater than 50% of sites in the cluster are methylated in the consensus methylation pattern.
Embodiment 168. The system of any one of embodiments 142-161, wherein greater than 25% of sites in the cluster are methylated in the consensus methylation pattern.
Embodiment 169. The system of any one of embodiments 142-168, wherein the plurality of sequence reads is obtained from whole-genome methyl sequencing (WGMS) or next-generation sequencing (NGS).
Embodiment 170. The system of any one of embodiments 142-169, wherein the plurality of sequence reads includes paired-end sequence reads.
Embodiment 171. The system of embodiment 170, wherein the consensus methylation pattern and CCF are determined based on paired-end sequence reads corresponding to the cluster.
Embodiment 172. The system of any one of embodiments 142-169, wherein the plurality of sequence reads includes unpaired sequence reads.
Embodiment 173. The system of any one of embodiments 142-172, wherein the one or more computer program instructions when executed by the one or more processors are further configured to, prior to determining the consensus methylation pattern and generating the CCF: demultiplex, using the one or more processors, sequence reads from the plurality of sequence reads.
Embodiment 174. The system of any one of embodiments 142-173, wherein the one or more computer program instructions when executed by the one or more processors are further configured to, prior to determining the consensus methylation pattern and generating the CCF: perform, using the one or more processors, three-letter alignment of sequence reads from the plurality to a reference genome.
Embodiment 175. The system of any one of embodiments 142-174, wherein the one or more computer program instructions when executed by the one or more processors are further configured to, prior to determining the consensus methylation pattern and generating the CCF: exclude, using the one or more processors, sequencing reads from the plurality that failed to undergo cytosine conversion.
Embodiment 176. The system of any one of embodiments 142-175, wherein the one or more computer program instructions when executed by the one or more processors are further configured to, prior to determining the consensus methylation pattern and generating the CCF: exclude, using the one or more processors, sequence reads with a base other than cytosine or thymine at a first position of at least one of the CpG dinucleotides.
Embodiment 177. The system of any one of embodiments 142-176, wherein the one or more computer program instructions when executed by the one or more processors are further configured to, prior to determining the consensus methylation pattern and generating the CCF: exclude, using the one or more processors, sequence reads with a base quality below a threshold base quality.
Embodiment 178. The system of any one of embodiments 142-177, wherein the consensus methylation pattern and CCF are determined and generated based on sequence reads that cover a plurality of CpG dinucleotides in the cluster.
Embodiment 179. The system of embodiment 178, wherein the consensus methylation pattern and CCF are determined based on sequence reads that cover at least 50% of CpG dinucleotides in the cluster.
Embodiment 180. The system of embodiment 178, wherein the consensus methylation pattern and CCF are determined based on sequence reads that cover at least 90% of CpG dinucleotides in the cluster.
Embodiment 181. The system of embodiment 178, wherein the consensus methylation pattern and CCF are determined based on sequence reads that cover all CpG dinucleotides in the cluster.
Embodiment 182. The system of any one of embodiments 142-181, wherein the plurality of nucleic acid fragments has undergone cytosine conversion by bisulfite treatment.
Embodiment 183. The system of any one of embodiments 142-181, wherein the plurality of nucleic acid fragments has undergone cytosine conversion by TET-assisted bisulfite treatment, TET-assisted pyridine borane treatment, oxidative bisulfite treatment, or APOBEC treatment.
Embodiment 184. A non-transitory computer readable storage medium comprising one or more programs executable by one or more computer processors for performing a method, comprising:
   obtaining a plurality of sequence reads from a plurality of nucleic acid fragments exhibiting cytosine conversion;
   determining, using the one or more processors, a consensus methylation pattern for a cluster of two or more CpG dinucleotides at a genomic locus, wherein the consensus methylation pattern represents each CpG dinucleotide in the cluster for which methylation was detected in at least one sequence read from a plurality of sequence reads;
   generating, using the one or more processors, a cluster consensus fraction (CCF) for the cluster, wherein the CCF represents a fraction of sequence reads corresponding to the cluster that show the consensus methylation pattern out of a total number of sequence reads from the plurality corresponding to the cluster; and
   detecting, by the processor, one or more of the methylation level or the unmethylation level of the cluster based on the CCF.
Embodiment 185. The non-transitory computer readable storage medium of embodiment 184, wherein the plurality of sequence reads is obtained from a plurality of nucleic acid fragments that has undergone cytosine conversion.
Embodiment 186. The non-transitory computer readable storage medium of embodiment 184 or embodiment 185, wherein the CCF is at or above a threshold or reference value, and wherein the method further comprises: detecting, using the one or more processors, presence of cancer nucleic acids in the plurality of nucleic acid fragments, based at least in part on the CCF being at or above the threshold or reference value.
Embodiment 187. The non-transitory computer readable storage medium of embodiment 184 or embodiment 185, wherein the CCF is at or above a threshold or reference value, and wherein the method further comprises: detecting, using the one or more processors, absence of cancer nucleic acids in the plurality of nucleic acid fragments, based at least in part on the CCF being below the threshold or reference value.
Embodiment 188. The non-transitory computer readable storage medium of any one of embodiments 184-187, wherein the method further comprises:
   determining, using the one or more processors, a consensus methylation pattern for more than one cluster of two or more CpG dinucleotides; and
   generating, using the one or more processors, a cluster consensus fraction (CCF) more than one cluster.
Embodiment 189. The non-transitory computer readable storage medium of embodiment 188, wherein the more than one cluster corresponds to more than one genomic locus.
Embodiment 190. The non-transitory computer readable storage medium of embodiment 188 or embodiment 189, wherein the method comprises determining a consensus methylation pattern and generating a CCF for more than 1,000 clusters.
Embodiment 191. The non-transitory computer readable storage medium of embodiment 188 or embodiment 189, wherein the method comprises determining a consensus methylation pattern and generating a CCF for between 10 and 100,000 clusters.
Embodiment 192. The non-transitory computer readable storage medium of embodiment 188 or embodiment 189, wherein the method comprises determining a consensus methylation pattern and generating a CCF for up to 1 million clusters.
Embodiment 193. The non-transitory computer readable storage medium of any one of embodiments 184-192, wherein the plurality of sequence reads comprises at least 100 sequence reads corresponding to the cluster.
Embodiment 194. The non-transitory computer readable storage medium of embodiment 193, wherein the plurality of sequence reads comprises at least 1000 sequence reads corresponding to the cluster.
Embodiment 195. The non-transitory computer readable storage medium of any one of embodiments 184-192, wherein the plurality of sequence reads comprises between 1 and 5 sequence reads corresponding to the cluster.
Embodiment 196. The non-transitory computer readable storage medium of any one of embodiments 184-195, wherein at least one CpG dinucleotide in the cluster is unmethylated in the consensus methylation pattern.
Embodiment 197. The non-transitory computer readable storage medium of any one of embodiments 184-196, wherein at least one CpG dinucleotide in the cluster is methylated in the consensus methylation pattern.
Embodiment 198. The non-transitory computer readable storage medium of any one of embodiments 184-197, wherein at least one cluster comprises two or more CpG dinucleotides.
Embodiment 199. The non-transitory computer readable storage medium of embodiment 198, wherein each cluster comprises two or more CpG dinucleotides.
Embodiment 200. The non-transitory computer readable storage medium of any one of embodiments 184-197, wherein at least one cluster comprises five or more CpG dinucleotides.
Embodiment 201. The non-transitory computer readable storage medium of embodiment 200, wherein each cluster comprises five or more CpG dinucleotides.
Embodiment 202. The non-transitory computer readable storage medium of any one of embodiments 184-201, wherein at least one cluster comprises six or more CpG dinucleotides.
Embodiment 203. The non-transitory computer readable storage medium of any one of embodiments 184-202, wherein all sites in the cluster except one are unmethylated in the consensus methylation pattern.
Embodiment 204. The non-transitory computer readable storage medium of any one of embodiments 184-202, wherein all sites in the cluster except two are unmethylated in the consensus methylation pattern.
Embodiment 205. The non-transitory computer readable storage medium of any one of embodiments 184-202, wherein at most 1 site in the cluster is methylated in the consensus methylation pattern.
Embodiment 206. The non-transitory computer readable storage medium of any one of embodiments 184-202, wherein at most 2 sites in the cluster are methylated in the consensus methylation pattern.
Embodiment 207. The non-transitory computer readable storage medium of any one of embodiments 184-202, wherein at most 10% of sites in the cluster are methylated in the consensus methylation pattern.
Embodiment 208. The non-transitory computer readable storage medium of any one of embodiments 184-202, wherein at most 25% of sites in the cluster are methylated in the consensus methylation pattern.
Embodiment 209. The non-transitory computer readable storage medium of any one of embodiments 184-204, wherein greater than 75% of sites in the cluster are methylated in the consensus methylation pattern.
Embodiment 210. The non-transitory computer readable storage medium of any one of embodiments 184-204, wherein greater than 50% of sites in the cluster are methylated in the consensus methylation pattern.
Embodiment 211. The non-transitory computer readable storage medium of any one of embodiments 184-204, wherein greater than 25% of sites in the cluster are methylated in the consensus methylation pattern.
Embodiment 212. The non-transitory computer readable storage medium of any one of embodiments 184-211, wherein the plurality of sequence reads is obtained from whole-genome methyl sequencing (WGMS) or next-generation sequencing (NGS).
Embodiment 213. The non-transitory computer readable storage medium of any one of embodiments 184-212, wherein the plurality of sequence reads includes paired-end sequence reads.
Embodiment 214. The non-transitory computer readable storage medium of embodiment 213, wherein the consensus methylation pattern and CCF are determined based on paired-end sequence reads corresponding to the cluster.
Embodiment 215. The non-transitory computer readable storage medium of any one of embodiments 184-212, wherein the plurality of sequence reads includes unpaired sequence reads.
Embodiment 216. The non-transitory computer readable storage medium of any one of embodiments 184-215, wherein the method comprises, prior to determining the consensus methylation pattern and generating the CCF: demultiplexing, using the one or more processors, sequence reads from the plurality of sequence reads.
Embodiment 217. The non-transitory computer readable storage medium of any one of embodiments 184-216, wherein the method comprises, prior to determining the consensus methylation pattern and generating the CCF: performing, using the one or more processors, three-letter alignment of sequence reads from the plurality to a reference genome.
Embodiment 218. The non-transitory computer readable storage medium of any one of embodiments 184-217, wherein the method comprises, prior to determining the consensus methylation pattern and generating the CCF: excluding, using the one or more processors, sequencing reads from the plurality that failed to undergo cytosine conversion.
Embodiment 219. The non-transitory computer readable storage medium of any one of embodiments 184-218, wherein the method comprises, prior to determining the consensus methylation pattern and generating the CCF: excluding, using the one or more processors, sequence reads with a base other than cytosine or thymine at a first position of at least one of the CpG dinucleotides.
Embodiment 220. The non-transitory computer readable storage medium of any one of embodiments 184-219, wherein the method comprises, prior to determining the consensus methylation pattern and generating the CCF: excluding, using the one or more processors, sequence reads with a base quality below a threshold base quality.
Embodiment 221. The non-transitory computer readable storage medium of any one of embodiments 184-220, wherein the consensus methylation pattern and CCF are determined and generated based on sequence reads that cover a plurality of CpG dinucleotides in the cluster.
Embodiment 222. The non-transitory computer readable storage medium of embodiment 221, wherein the consensus methylation pattern and CCF are determined based on sequence reads that cover at least 50% of CpG dinucleotides in the cluster.
Embodiment 223. The non-transitory computer readable storage medium of embodiment 221, wherein the consensus methylation pattern and CCF are determined based on sequence reads that cover at least 90% of CpG dinucleotides in the cluster.
Embodiment 224. The non-transitory computer readable storage medium of embodiment 221, wherein the consensus methylation pattern and CCF are determined based on sequence reads that cover all CpG dinucleotides in the cluster.
Embodiment 225. The non-transitory computer readable storage medium of any one of embodiments 184-224, wherein the plurality of nucleic acid fragments has undergone cytosine conversion by bisulfite treatment.
Embodiment 226. The non-transitory computer readable storage medium of any one of embodiments 184-224, wherein the plurality of nucleic acid fragments has undergone cytosine conversion by TET-assisted bisulfite treatment, TET-assisted pyridine borane treatment, oxidative bisulfite treatment, or APOBEC treatment.
Embodiment 227. A method of detecting one or more of a methylation level or an unmethylation level of a cluster of two or more CpG dinucleotides in a sample from a subject, comprising:
   obtaining a plurality of nucleic acid fragments from the sample;
   amplifying the plurality of nucleic acid fragments;
   sequencing, by a sequencer, the plurality of amplified nucleic acid fragments to obtain a plurality of sequence reads, wherein at least the plurality of amplified nucleic acid fragments has undergone cytosine conversion, and wherein the plurality of nucleic acid fragments corresponds to a genomic locus comprising a cluster of two or more CpG dinucleotides;
   determining, by a processor, a consensus unmethylation pattern for the cluster, wherein the consensus unmethylation pattern represents each CpG dinucleotide in the cluster for which methylation was not detected based on the cytosine conversion in at least one sequence read from the plurality of sequence reads;
   generating, by a processor, a cluster consensus fraction (CCF) for the cluster, wherein the CCF represents a fraction of sequence reads corresponding to the cluster that show the consensus unmethylation pattern out of a total number of sequence reads from the plurality corresponding to the cluster;
   detecting one or more of the methylation level or the unmethylation level of the cluster based on the CCF; and
   generating a genomic profile for the subject based on the detected methylation level, the detected unmethylation level, or both.
Embodiment 228. A method of detecting one or more of a methylation level or an unmethylation level of a cluster of two or more CpG dinucleotides from a sample, comprising:
   obtaining a plurality of sequence reads from a plurality of nucleic acid fragments exhibiting cytosine conversion;
   determining, by a processor, a consensus unmethylation pattern for a cluster of two or more CpG dinucleotides at a locus, wherein the consensus unmethylation pattern represents each CpG dinucleotide in the cluster for which methylation was not detected;
   generating, by a processor, a cluster consensus fraction (CCF) for the cluster, wherein the CCF represents a fraction of sequence reads corresponding to the cluster that show the consensus unmethylation pattern out of a total number of sequence reads from the plurality corresponding to the cluster; and
   detecting, by the processor, one or more of the methylation level or the unmethylation level of the cluster based on the CCF.
Embodiment 229. The method of embodiment 228, wherein the CCF represents a fraction of sequence reads corresponding to the cluster that show the consensus unmethylation pattern out of a total number of sequence reads from the plurality corresponding to the cluster based on the cytosine conversion in at least one sequence read from the plurality of sequence reads.
Embodiment 230. A method of detecting one or more of a methylation level or an unmethylation level of a cluster of two or more CpG dinucleotides, comprising:
   sequencing, by a sequencer, the plurality of nucleic acid fragments to obtain the plurality of sequence reads;
   determining, by a processor, a consensus unmethylation pattern for the cluster, wherein the consensus unmethylation pattern represents each CpG dinucleotide in the cluster for which methylation was not detected in at least one sequence read from the plurality based on the cytosine conversion;
   generating, by a processor, a cluster consensus fraction (CCF) for the cluster, wherein the CCF represents a fraction of sequence reads corresponding to the cluster that show the consensus unmethylation pattern out of a total number of sequence reads from the plurality corresponding to the cluster, thereby detecting one or more of the methylation level or the unmethylation level of the cluster;
   detecting, by the processor, one or more of the methylation level or the unmethylation level of the cluster based on the CCF.
Embodiment 231. The method of any one of embodiments 227-230, wherein the CCF is below a threshold or reference value, and the method further comprises: detecting presence of cancer nucleic acids in the plurality of nucleic acid fragments, based at least in part on the CCF being below the threshold or reference value.
Embodiment 232. The method of any one of embodiments 227-230, wherein the CCF is at or above a threshold or reference value, and the method further comprises: detecting absence of cancer nucleic acids in the plurality of nucleic acid fragments, based at least in part on the CCF being at or above the threshold or reference value.
Embodiment 233. The method of any one of embodiments 227-232, comprising determining a consensus methylation pattern and CCF for more than one cluster.
Embodiment 234. The method of embodiment 233, wherein the more than one cluster corresponds to more than one genomic locus.
Embodiment 235. The method of embodiment 233 or embodiment 234, comprising determining a consensus methylation pattern and CCF for more than 1,000 clusters.
Embodiment 236. The method of embodiment 233 or embodiment 234, comprising determining a consensus methylation pattern and CCF for between 10 and 100,000 clusters.
Embodiment 237. The method of any one of embodiments 227-236, comprising determining a consensus methylation pattern and CCF for up to 1 million clusters.
Embodiment 238. The method of any one of embodiments 227-237, wherein the plurality of sequence reads comprises at least 100 sequence reads corresponding to the cluster.
Embodiment 239. The method of embodiment 238, wherein the plurality of sequence reads comprises at least 1000 sequence reads corresponding to the cluster.
Embodiment 240. The method of any one of embodiments 227-237, wherein the plurality of sequence reads comprises between 1 and 5 sequence reads corresponding to the cluster.
Embodiment 241. The method of any one of embodiments 227-240, wherein at least one CpG dinucleotide in the cluster is methylated in the consensus methylation pattern.
Embodiment 242. The method of any one of embodiments 227-241, wherein at least one CpG dinucleotide in the cluster is unmethylated in the consensus methylation pattern.
Embodiment 243. The method of any one of embodiments 227-242, wherein at least one cluster comprises two or more CpG dinucleotides.
Embodiment 244. The method of embodiment 243, wherein each cluster comprises two or more CpG dinucleotides.
Embodiment 245. The method of any one of embodiments 227-244, wherein at least one cluster comprises five or more CpG dinucleotides.
Embodiment 246. The method of embodiment 245, wherein each cluster comprises five or more CpG dinucleotides.
Embodiment 247. The method of any one of embodiments 227-246, wherein at least one cluster comprises six or more CpG dinucleotides.
Embodiment 248. The method of any one of embodiments 227-247, wherein all sites in the cluster except one are methylated in the consensus methylation pattern.
Embodiment 249. The method of any one of embodiments 227-247, wherein all sites in the cluster except two are methylated in the consensus methylation pattern.
Embodiment 250. The method of any one of embodiments 227-247, wherein at most 1 site in the cluster is unmethylated in the consensus methylation pattern.
Embodiment 251. The method of any one of embodiments 227-247, wherein at most 2 sites in the cluster are unmethylated in the consensus methylation pattern.
Embodiment 252. The method of any one of embodiments 227-247, wherein at most 10% of sites in the cluster are unmethylated in the consensus methylation pattern.
Embodiment 253. The method of any one of embodiments 227-247, wherein at most 25% of sites in the cluster are unmethylated in the consensus methylation pattern.
Embodiment 254. The method of any one of embodiments 227-249, wherein greater than 75% of sites in the cluster are unmethylated in the consensus methylation pattern.
Embodiment 255. The method of any one of embodiments 227-249, wherein greater than 50% of sites in the cluster are unmethylated in the consensus methylation pattern.
Embodiment 256. The method of any one of embodiments 227-249, wherein greater than 25% of sites in the cluster are unmethylated in the consensus methylation pattern.
Embodiment 257. The method of any one of embodiments 227-256, wherein the plurality of sequence reads is obtained from whole-genome methyl sequencing (WGMS) or next-generation sequencing (NGS).
Embodiment 258. The method of any one of embodiments 227-257, wherein the plurality of sequence reads includes paired-end sequence reads.
Embodiment 259. The method of embodiment 258, wherein the consensus methylation pattern and CCF are determined based on paired-end sequence reads corresponding to the cluster.
Embodiment 260. The method of any one of embodiments 227-257, wherein the plurality of sequence reads includes unpaired sequence reads.
Embodiment 261. The method of any one of embodiments 227-260, further comprising, prior to determining the consensus methylation pattern and CCF, demultiplexing sequence reads from the plurality of sequence reads.
Embodiment 262. The method of any one of embodiments 227-261, further comprising, prior to determining the consensus methylation pattern and CCF, performing three-letter alignment of sequence reads from the plurality to a reference genome.
Embodiment 263. The method of any one of embodiments 227-262, further comprising, prior to determining the consensus methylation pattern and CCF, excluding sequencing reads from the plurality that failed to undergo cytosine conversion.
Embodiment 264. The method of any one of embodiments 227-263, further comprising, prior to determining the consensus methylation pattern and CCF, excluding sequence reads with a base other than cytosine or thymine at a first position of at least one of the CpG dinucleotides.
Embodiment 265. The method of any one of embodiments 227-264, further comprising, prior to determining the consensus methylation pattern and CCF, excluding sequence reads with a base quality below a threshold base quality.
Embodiment 266. The method of any one of embodiments 227-265, wherein the consensus methylation pattern and CCF are determined based on sequence reads that cover a plurality of CpG dinucleotides in the cluster.
Embodiment 267. The method of embodiment 266, wherein the consensus methylation pattern and CCF are determined based on sequence reads that cover at least 50% of CpG dinucleotides in the cluster.
Embodiment 268. The method of embodiment 266, wherein the consensus methylation pattern and CCF are determined based on sequence reads that cover at least 90% of CpG dinucleotides in the cluster.
Embodiment 269. The method of embodiment 266, wherein the consensus methylation pattern and CCF are determined based on sequence reads that cover all CpG dinucleotides in the cluster.
Embodiment 270. The method of any one of embodiments 227-269, wherein the plurality of nucleic acid fragments has undergone cytosine conversion by bisulfite treatment.
Embodiment 271. The method of any one of embodiments 227-269, wherein the plurality of nucleic acid fragments has undergone cytosine conversion by TET-assisted bisulfite treatment, TET-assisted pyridine borane treatment, oxidative bisulfite treatment, or APOBEC treatment.
Embodiment 272. The method of any one of embodiments 227-269, further comprising, prior to obtaining the plurality of sequence reads, treating a plurality of nucleic acids or nucleic acid fragments with bisulfite.
Embodiment 273. The method of any one of embodiments 227-269, further comprising, prior to obtaining the plurality of sequence reads, treating a plurality of nucleic acids or nucleic acid fragments with TET-assisted bisulfite treatment, TET-assisted pyridine borane treatment, oxidative bisulfite treatment, or APOBEC treatment.
Embodiment 274. The method of any one of embodiments 227-273, further comprising, prior to obtaining the plurality of sequence reads, subjecting a plurality of nucleic acids to fragmentation.
Embodiment 275. The method of any one of embodiments 227-274, further comprising, prior to obtaining the plurality of sequence reads, selectively enriching for a plurality of nucleic acids or nucleic acid fragments corresponding to a genomic locus that comprises a cluster of two or more CpG dinucleotides to produce an enriched sample.
Embodiment 276. The method of any one of embodiments 227-275, wherein the amplification of the plurality of nucleic acids or nucleic acid fragments is performed by polymerase chain reaction (PCR).
Embodiment 277. The method of any one of embodiments 227-276, further comprising, prior to obtaining the plurality of sequence reads, isolating the plurality of nucleic acids from a sample.
Embodiment 278. The method of embodiment 277, wherein the sample comprises tumor cells and/or tumor nucleic acids.
Embodiment 279. The method of embodiment 278, wherein the sample further comprises non-tumor cells and/or non-tumor nucleic acids.
Embodiment 280. The method of embodiment 279, wherein the sample comprises a fraction of tumor nucleic acids that is less than 1% of total nucleic acids.
Embodiment 281. The method of embodiment 279, wherein the sample comprises a fraction of tumor nucleic acids that is less than 0.1% of total nucleic acids.
Embodiment 282. The method of any one of embodiments 279-281, wherein the sample comprises a fraction of tumor nucleic acids that is at least 0.01% of total nucleic acids.
Embodiment 283. The method of any one of embodiments 277-282, wherein the sample comprises tumor cell-free DNA (cfDNA), circulating cell-free DNA (ccfDNA), or circulating tumor DNA (ctDNA).
Embodiment 284. The method of any one of embodiments 277-282, wherein the sample comprises fluid, cells, or tissue.
Embodiment 285. The method of embodiment 284, wherein the sample comprises blood or plasma.
Embodiment 286. The method of any one of embodiments 277-282, wherein the sample comprises a tumor biopsy or a circulating tumor cell.
Embodiment 287. The method of any one of embodiments 227-286, wherein the sample is a tissue sample, and the method further comprises: subjecting a plurality of nucleic acid molecules in the tissue to fragmentation to create the plurality of nucleic acid fragments.
Embodiment 288. The method of embodiment 287, further comprising: ligating one or more adapters onto one or more nucleic acid fragments from the plurality of nucleic acid fragments prior to amplifying the plurality of nucleic acid fragments.
Embodiment 289. A system, comprising:
   one or more processors; and
   a memory configured to store one or more computer program instructions, wherein the one or more computer program instructions when executed by the one or more processors are configured to:
      determine, using the one or more processors, a consensus unmethylation pattern for a cluster of two or more CpG dinucleotides at a genomic locus, wherein the consensus unmethylation pattern represents each CpG dinucleotide in the cluster for which methylation was not detected in at least one sequence read from a plurality of sequence reads obtained from a plurality of nucleic acid fragments that has undergone cytosine conversion; and
      generate, using the one or more processors, a cluster consensus fraction (CCF) for the cluster, wherein the CCF represents a fraction of sequence reads corresponding to the cluster that show the consensus unmethylation pattern out of a total number of sequence reads from the plurality corresponding to the cluster.
Embodiment 290. The system of embodiment 289, wherein the CCF is at or above a threshold or reference value, and wherein the one or more computer program instructions when executed by the one or more processors are further configured to: detect, using the one or more processors, absence of cancer nucleic acids in the plurality of nucleic acid fragments, based at least in part on the CCF being at or above the threshold or reference value.
Embodiment 291. The system of embodiment 289, wherein the CCF is below a threshold or reference value, and wherein the one or more computer program instructions when executed by the one or more processors are further configured to: detect, using the one or more processors, presence of cancer nucleic acids in the plurality of nucleic acid fragments, based at least in part on the CCF being below the threshold or reference value.
Embodiment 292. The system of any one of embodiments 289-291, wherein the one or more computer program instructions when executed by the one or more processors are further configured to:
   determine, using the one or more processors, a consensus methylation pattern for more than one cluster of two or more CpG dinucleotides; and
   generate, using the one or more processors, a cluster consensus fraction (CCF) for more than one cluster.
Embodiment 293. The system of embodiment 292, wherein the more than one cluster corresponds to more than one genomic locus.
Embodiment 294. The system of embodiment 292 or embodiment 293, wherein the one or more computer program instructions when executed by the one or more processors are configured to determine a consensus methylation pattern and generate a CCF for more than 1,000 clusters.
Embodiment 295. The system of embodiment 292 or embodiment 293, wherein the one or more computer program instructions when executed by the one or more processors are configured to determine a consensus methylation pattern and generate a CCF for between 10 and 100,000 clusters.
Embodiment 296. The system of embodiment 292 or embodiment 293, wherein the one or more computer program instructions when executed by the one or more processors are configured to determine a consensus methylation pattern and generate a CCF for up to 1 million clusters.
Embodiment 297. The system of any one of embodiments 289-296, wherein the plurality of sequence reads comprises at least 100 sequence reads corresponding to the cluster.
Embodiment 298. The system of embodiment 297, wherein the plurality of sequence reads comprises at least 1000 sequence reads corresponding to the cluster.
Embodiment 299. The system of any one of embodiments 289-296, wherein the plurality of sequence reads comprises between 1 and 5 sequence reads corresponding to the cluster.
Embodiment 300. The system of any one of embodiments 289-299, wherein at least one CpG dinucleotide in the cluster is unmethylated in the consensus methylation pattern.
Embodiment 301. The system of any one of embodiments 289-300, wherein at least one CpG dinucleotide in the cluster is unmethylated in the consensus methylation pattern.
Embodiment 302. The system of any one of embodiments 289-301, wherein at least one cluster comprises two or more CpG dinucleotides.
Embodiment 303. The system of embodiment 302, wherein each cluster comprises two or more CpG dinucleotides.
Embodiment 304. The system of any one of embodiments 289-301, wherein at least one cluster comprises five or more CpG dinucleotides.
Embodiment 305. The system of embodiment 304, wherein each cluster comprises five or more CpG dinucleotides.
Embodiment 306. The system of any one of embodiments 289-305, wherein at least one cluster comprises six or more CpG dinucleotides.
Embodiment 307. The system of any one of embodiments 289-306, wherein all sites in the cluster except one are methylated in the consensus methylation pattern.
Embodiment 308. The system of any one of embodiments 289-306, wherein all sites in the cluster except two are methylated in the consensus methylation pattern.
Embodiment 309. The system of any one of embodiments 289-306, wherein at most 1 site in the cluster is unmethylated in the consensus methylation pattern.
Embodiment 310. The system of any one of embodiments 289-306, wherein at most 2 sites in the cluster are unmethylated in the consensus methylation pattern.
Embodiment 311. The system of any one of embodiments 289-306, wherein at most 10% of sites in the cluster are unmethylated in the consensus methylation pattern.
Embodiment 312. The system of any one of embodiments 289-306, wherein at most 25% of sites in the cluster are unmethylated in the consensus methylation pattern.
Embodiment 313. The system of any one of embodiments 289-312, wherein greater than 75% of sites in the cluster are unmethylated in the consensus methylation pattern.
Embodiment 314. The system of any one of embodiments 289-312, wherein greater than 50% of sites in the cluster are unmethylated in the consensus methylation pattern.
Embodiment 315. The system of any one of embodiments 289-312, wherein greater than 25% of sites in the cluster are unmethylated in the consensus methylation pattern.
Embodiment 316. The system of any one of embodiments 289-315, wherein the plurality of sequence reads is obtained from whole-genome methyl sequencing (WGMS) or next-generation sequencing (NGS).
Embodiment 317. The system of any one of embodiments 289-316, wherein the plurality of sequence reads includes paired-end sequence reads.
Embodiment 318. The system of embodiment 317, wherein the consensus methylation pattern and CCF are determined based on paired-end sequence reads corresponding to the cluster.
Embodiment 319. The system of any one of embodiments 289-316, wherein the plurality of sequence reads includes unpaired sequence reads.
Embodiment 320. The system of any one of embodiments 289-319, wherein the one or more computer program instructions when executed by the one or more processors are further configured to, prior to determining the consensus methylation pattern and generating the CCF: demultiplex, using the one or more processors, sequence reads from the plurality of sequence reads.
Embodiment 321. The system of any one of embodiments 289-320, wherein the one or more computer program instructions when executed by the one or more processors are further configured to, prior to determining the consensus methylation pattern and generating the CCF: perform, using the one or more processors, three-letter alignment of sequence reads from the plurality to a reference genome.
Embodiment 322. The system of any one of embodiments 289-321, wherein the one or more computer program instructions when executed by the one or more processors are further configured to, prior to determining the consensus methylation pattern and generating the CCF: exclude, using the one or more processors, sequencing reads from the plurality that failed to undergo cytosine conversion.
Embodiment 323. The system of any one of embodiments 289-322, wherein the one or more computer program instructions when executed by the one or more processors are further configured to, prior to determining the consensus methylation pattern and generating the CCF: exclude, using the one or more processors, sequence reads with a base other than cytosine or thymine at a first position of at least one of the CpG dinucleotides.
Embodiment 324. The system of any one of embodiments 289-323, wherein the one or more computer program instructions when executed by the one or more processors are further configured to, prior to determining the consensus methylation pattern and generating the CCF: exclude, using the one or more processors, sequence reads with a base quality below a threshold base quality.
Embodiment 325. The system of any one of embodiments 289-324, wherein the consensus methylation pattern and CCF are determined and generated based on sequence reads that cover a plurality of CpG dinucleotides in the cluster.
Embodiment 326. The system of embodiment 325, wherein the consensus methylation pattern and CCF are determined based on sequence reads that cover at least 50% of CpG dinucleotides in the cluster.
Embodiment 327. The system of embodiment 325, wherein the consensus methylation pattern and CCF are determined based on sequence reads that cover at least 90% of CpG dinucleotides in the cluster.
Embodiment 328. The system of embodiment 325, wherein the consensus methylation pattern and CCF are determined based on sequence reads that cover all CpG dinucleotides in the cluster.
Embodiment 329. The system of any one of embodiments 289-328, wherein the plurality of nucleic acid fragments has undergone cytosine conversion by bisulfite treatment.
Embodiment 330. The system of any one of embodiments 289-328, wherein the plurality of nucleic acid fragments has undergone cytosine conversion by TET-assisted bisulfite treatment, TET-assisted pyridine borane treatment, oxidative bisulfite treatment, or APOBEC treatment.
Embodiment 331. A non-transitory computer readable storage medium comprising one or more programs executable by one or more computer processors for performing a method, comprising:
   obtaining a plurality of sequence reads from a plurality of nucleic acid fragments exhibiting cytosine conversion;
   determining, using the one or more processors, a consensus unmethylation pattern for a cluster of two or more CpG dinucleotides at a locus, wherein the consensus unmethylation pattern represents each CpG dinucleotide in the cluster for which methylation was not detected in at least one sequence read from a plurality of sequence reads; and
   generating, using the one or more processors, a cluster consensus fraction (CCF) for the cluster, wherein the CCF represents a fraction of sequence reads corresponding to the cluster that show the consensus unmethylation pattern out of a total number of sequence reads from the plurality corresponding to the cluster; and
   detecting, by the processor, one or more of a methylation level or an unmethylation level of the cluster based on the CCF.
Embodiment 332. The non-transitory computer readable storage medium of embodiment 331, wherein the plurality of sequence reads is obtained from a plurality of nucleic acid fragments that has undergone cytosine conversion.
Embodiment 333. The non-transitory computer readable storage medium of embodiment 331 or embodiment 332, wherein the CCF is at or above a threshold or reference value, and wherein the method further comprises: detecting, using the one or more processors, absence of cancer nucleic acids in the plurality of nucleic acid fragments, based at least in part on the CCF being at or above the threshold or reference value.
Embodiment 334. The non-transitory computer readable storage medium of embodiment 331 or embodiment 332, wherein the CCF is at or above a threshold or reference value, and wherein the method further comprises: detecting, using the one or more processors, presence of cancer nucleic acids in the plurality of nucleic acid fragments, based at least in part on the CCF being below the threshold or reference value.
Embodiment 335. The non-transitory computer readable storage medium of any one of embodiments 331-334, wherein the method further comprises:
   determining, using the one or more processors, a consensus methylation pattern for more than one cluster of two or more CpG dinucleotides; and
   generating, using the one or more processors, a cluster consensus fraction (CCF) more than one cluster.
Embodiment 336. The non-transitory computer readable storage medium of embodiment 335, wherein the more than one cluster corresponds to more than one genomic locus.
Embodiment 337. The non-transitory computer readable storage medium of embodiment 335 or embodiment 336, wherein the method comprises determining a consensus methylation pattern and generating a CCF for more than 1,000 clusters.
Embodiment 338. The non-transitory computer readable storage medium of embodiment 335 or embodiment 336, wherein the method comprises determining a consensus methylation pattern and generating a CCF for between 10 and 100,000 clusters.
Embodiment 339. The non-transitory computer readable storage medium of embodiment 335 or embodiment 336, wherein the method comprises determining a consensus methylation pattern and generating a CCF for up to 1 million clusters.
Embodiment 340. The non-transitory computer readable storage medium of any one of embodiments 331-339, wherein the plurality of sequence reads comprises at least 100 sequence reads corresponding to the cluster.
Embodiment 341. The non-transitory computer readable storage medium of embodiment 340, wherein the plurality of sequence reads comprises at least 1000 sequence reads corresponding to the cluster.
Embodiment 342. The non-transitory computer readable storage medium of any one of embodiments 331-339, wherein the plurality of sequence reads comprises between 1 and 5 sequence reads corresponding to the cluster.
Embodiment 343. The non-transitory computer readable storage medium of any one of embodiments 331-342, wherein at least one CpG dinucleotide in the cluster is methylated in the consensus methylation pattern.
Embodiment 344. The non-transitory computer readable storage medium of any one of embodiments 331-343, wherein at least one CpG dinucleotide in the cluster is unmethylated in the consensus methylation pattern.
Embodiment 345. The non-transitory computer readable storage medium of any one of embodiments 331-344, wherein at least one cluster comprises two or more CpG dinucleotides.
Embodiment 346. The non-transitory computer readable storage medium of embodiment 345, wherein each cluster comprises two or more CpG dinucleotides.
Embodiment 347. The non-transitory computer readable storage medium of any one of embodiments 331-344, wherein at least one cluster comprises five or more CpG dinucleotides.
Embodiment 348. The non-transitory computer readable storage medium of embodiment 347, wherein each cluster comprises five or more CpG dinucleotides.
Embodiment 349. The non-transitory computer readable storage medium of any one of embodiments 331-348, wherein at least one cluster comprises six or more CpG dinucleotides.
Embodiment 350. The non-transitory computer readable storage medium of any one of embodiments 331-349, wherein all sites in the cluster except one are methylated in the consensus methylation pattern.
Embodiment 351. The non-transitory computer readable storage medium of any one of embodiments 331-349, wherein all sites in the cluster except two are methylated in the consensus methylation pattern.
Embodiment 352. The non-transitory computer readable storage medium of any one of embodiments 331-349, wherein at most 1 site in the cluster is unmethylated in the consensus methylation pattern.
Embodiment 353. The non-transitory computer readable storage medium of any one of embodiments 331-349, wherein at most 2 sites in the cluster are unmethylated in the consensus methylation pattern.
Embodiment 354. The non-transitory computer readable storage medium of any one of embodiments 331-349, wherein at most 10% of sites in the cluster are unmethylated in the consensus methylation pattern.
Embodiment 355. The non-transitory computer readable storage medium of any one of embodiments 331-349, wherein at most 25% of sites in the cluster are unmethylated in the consensus methylation pattern.
Embodiment 356. The non-transitory computer readable storage medium of any one of embodiments 331-351, wherein greater than 75% of sites in the cluster are unmethylated in the consensus methylation pattern.
Embodiment 357. The non-transitory computer readable storage medium of any one of embodiments 331-351, wherein greater than 50% of sites in the cluster are unmethylated in the consensus methylation pattern.
Embodiment 358. The non-transitory computer readable storage medium of any one of embodiments 331-351, wherein greater than 25% of sites in the cluster are unmethylated in the consensus methylation pattern.
Embodiment 359. The non-transitory computer readable storage medium of any one of embodiments 331-358, wherein the plurality of sequence reads is obtained from whole-genome methyl sequencing (WGMS) or next-generation sequencing (NGS).
Embodiment 360. The non-transitory computer readable storage medium of any one of embodiments 331-359, wherein the plurality of sequence reads includes paired-end sequence reads.
Embodiment 361. The non-transitory computer readable storage medium of embodiment 360, wherein the consensus methylation pattern and CCF are determined based on paired-end sequence reads corresponding to the cluster.
Embodiment 362. The non-transitory computer readable storage medium of any one of embodiments 331-359, wherein the plurality of sequence reads includes unpaired sequence reads.
Embodiment 363. The non-transitory computer readable storage medium of any one of embodiments 331-362, wherein the method comprises, prior to determining the consensus methylation pattern and generating the CCF: demultiplexing, using the one or more processors, sequence reads from the plurality of sequence reads.
Embodiment 364. The non-transitory computer readable storage medium of any one of embodiments 331-363, wherein the method comprises, prior to determining the consensus methylation pattern and generating the CCF: performing, using the one or more processors, three-letter alignment of sequence reads from the plurality to a reference genome.
Embodiment 365. The non-transitory computer readable storage medium of any one of embodiments 331-364, wherein the method comprises, prior to determining the consensus methylation pattern and generating the CCF: excluding, using the one or more processors, sequencing reads from the plurality that failed to undergo cytosine conversion.
Embodiment 366. The non-transitory computer readable storage medium of any one of embodiments 331-365, wherein the method comprises, prior to determining the consensus methylation pattern and generating the CCF: excluding, using the one or more processors, sequence reads with a base other than cytosine or thymine at a first position of at least one of the CpG dinucleotides.
Embodiment 367. The non-transitory computer readable storage medium of any one of embodiments 331-366, wherein the method comprises, prior to determining the consensus methylation pattern and generating the CCF: excluding, using the one or more processors, sequence reads with a base quality below a threshold base quality.
Embodiment 368. The non-transitory computer readable storage medium of any one of embodiments 331-367, wherein the consensus methylation pattern and CCF are determined and generated based on sequence reads that cover a plurality of CpG dinucleotides in the cluster.
Embodiment 369. The non-transitory computer readable storage medium of embodiment 368, wherein the consensus methylation pattern and CCF are determined based on sequence reads that cover at least 50% of CpG dinucleotides in the cluster.
Embodiment 370. The non-transitory computer readable storage medium of embodiment 368, wherein the consensus methylation pattern and CCF are determined based on sequence reads that cover at least 90% of CpG dinucleotides in the cluster.
Embodiment 371. The non-transitory computer readable storage medium of embodiment 368, wherein the consensus methylation pattern and CCF are determined based on sequence reads that cover all CpG dinucleotides in the cluster.
Embodiment 372. The non-transitory computer readable storage medium of any one of embodiments 331-371, wherein the plurality of nucleic acid fragments has undergone cytosine conversion by bisulfite treatment.
Embodiment 373. The non-transitory computer readable storage medium of any one of embodiments 331-371, wherein the plurality of nucleic acid fragments has undergone cytosine conversion by TET-assisted bisulfite treatment, TET-assisted pyridine borane treatment, oxidative bisulfite treatment, or APOBEC treatment.

The disclosures of all publications, patents, and patent applications referred to herein are each hereby incorporated by reference in their entireties. To the extent that any reference incorporated by reference conflicts with the instant disclosure, the instant disclosure shall control.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Fragment Consensus-Based Approaches for Ultrasensitive Detection of Aberrant DNA Methylation

In early-stage cancers, ccfDNA often contains cancer-derived molecules at a frequency of 1 in 1,000 down to 1 in 100,000, presenting an obstacle to the application of many analytical methods. A similar challenge arises using other sample types where cancer DNA is present but at low quantities, including urine cell-free DNA, cerebrospinal fluid, and others. Sensitive detection of cancer signal at this level is likely necessary for the successful application of ccfDNA to detection of MRD and blood-based monitoring of early-stage cancer patients.

Dysregulation of gene expression is a hallmark of cancer, and one way of observing that in blood directly is by examining aberrant DNA methylation in ccfDNA. DNA methylation occurs at cytosines that are followed by guanine (CG dinucleotides, sometimes known as “CpG sites”). Analysis of DNA methylation can be performed by combining cytosine conversion and next-generation sequencing (NGS). These assays convert cytosine nucleotides to another base (C to T) depending on whether they are methylated or not, enabling a bioinformatic determination of methylation with single-base resolution. Two commonly used techniques for this are bisulfite sequencing and “Enzymatic Methyl-seq” (NEB product), which both convert unmethylated cytosines, while leaving methylated cytosines unconverted.

Reliable detection of cancer-driven changes in methylation requires very high levels of analytical sensitivity in clinically relevant ranges, e.g., 1 in 1,000 down to 1 in 1,000,000, using data from cytosine conversion assays or any other methylation assays with single-base resolution. However, there are several key analytical obstacles to achieving this goal. First, sequencing errors (e.g., using the Illumina platform) occur in the top end of this range, so measurement artifacts could appear as cancer signals. Second, methylation artifacts arise in cytosine conversion assays. In particular, some read positions have biased measurements due to alignments or library preparation. Some of these biases tend to be restricted to a subset of a measured DNA fragment (e.g., near fragment ends), but these biases can meaningfully impact background levels. Third, methylation sites across genomes have basal levels of methylation or non-methylation. As a result, healthy samples can have residual signal that makes them difficult to distinguish from cancer ccfDNA samples with low levels of cancer.

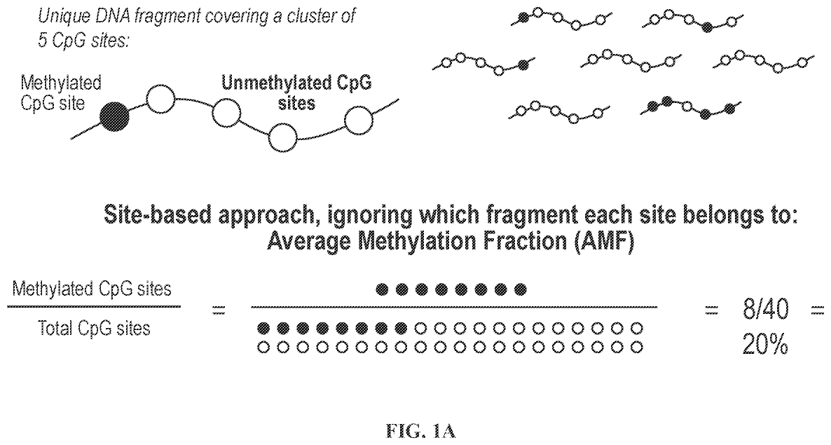

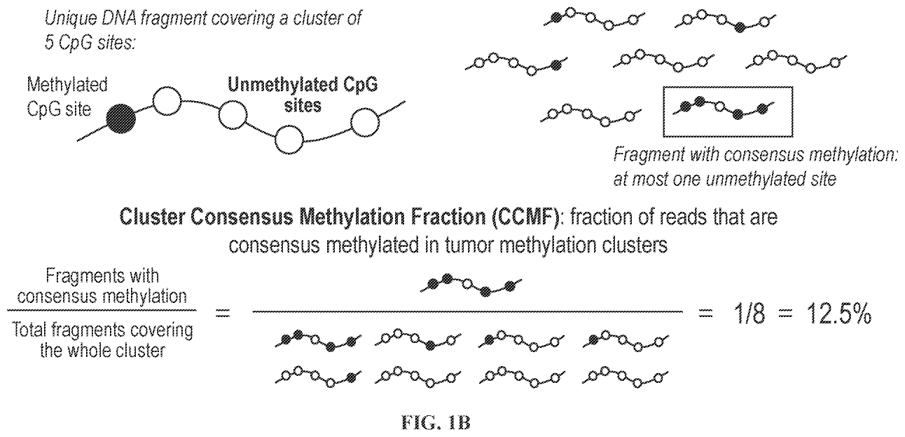

Previous attempts at addressing these problems have included identification of differential methylation regions (DMRs) and comparison of average methylation fraction (AMF) in these regions, as shown in FIG. 1A. These assays were found to be insufficient in enabling ultra-sensitive detection of cancer signals. Guo et al. (Nat. Genet. 2017 49:635-642) applied the concept of linkage disequilibrium to methylation and defined several read-based metrics to aid in detection and clustering of cancer in tissue and ccfDNA samples. These included Methyl-Haplotype Load, a score that rewards consecutively methylated or consecutively unmethylated sites. This approach was found to provide little to no improvement in analytical performance.

Liu et al. (Ann. Oncol. 2020 31:745-759) defined a concept of Methyl Variants, i.e., a set of 5 contiguous CG dinucleotides that are 0% or 100% methylated at high frequency in at least one known cancer sample (tissue biopsy) out of a dataset produced from a large cohort. Defining MVs as exactly 5 consecutive sites leads to a smaller number of potential sites than the methods of the present disclosure, which are more expansive and include a range of sizes and site counts. The methods disclosed herein define more regions, as well as regions that have more methylation regions. For example, some CpG clusters have more than 10 CpG sites.

This Example describes a “Cluster Consensus Fraction” (CCF) approach for detecting methylation levels. Using this approach was found to effectively increase the signal-to-background ratio by more than 100-fold, enabling ultrasensitive detection of methylation levels. In this case, a CCMF approach was used (assaying methylation rather than unmethylation).

### Materials and Methods

### Analysis Pipeline

Hybrid capture was performed using probes designed to enrich both methylated and unmethylated DNA strands using Twist fast Hyb wash reagents and optimized conditions. Cytosine conversion was performed with enzymatic methyl sequencing (EM-seq). DNA was from a cell line repository, and was sonicated to size of interest prior to library preparation.

After Illumina sequencing, the following workflow was used to compute consensus metrics. First, a determination of reads that overlap with a cluster of CG dinucleotides (“CpG cluster”, defined below), which also pass basic sequencing quality filtering and are properly paired, was made. Reads that did not cover all CG dinucleotides in the cluster were excluded. For each set of paired reads, base calls at each C within a CG dinucleotide were determined using a combination of the two paired end reads for positions that may be overlapping, which are the location of each methylation call from the DNA fragment. Reads that had unexpected bases, e.g., those other than an C (unconverted) or T (converted) on the “original strand”, the strand to which the first sequencing read was mapped, were excluded. These could be due to sequencing errors or true mutations (somatic or germline) and could confound the methylation analysis. Read pairs with base quality below a threshold for any base to be used in a methylation call were excluded.

For remaining read pairs, methylation calls were tabulated at each CG dinucleotide across the cluster: at the specified positions on the original strand, a C indicates the nucleotide was methylated and T indicates the nucleotide was not methylated. A consensus condition was applied across the set of methylation calls for each read pair. A consensus condition classified read status as a function of the number of total sites and the number of methylated sites in the cluster. Consensus conditions can include: perfect methylation (100% of sites are methylated), mismatch threshold methylation (at most a specific number of sites out of all sites are unmethylated, e.g., 1, 2, or higher), majority methylated (more than half of sites are methylated, scoring ties as zero or half credit), fractional threshold (at least a specific fraction of sites is methylated, i.e., any fraction between 0 and 1), or any of the above conditions but for unmethylated sites. Finally, data from multiple clusters were aggregated. Measurements from individual CpG clusters or collections of CpG clusters, using a specified consensus condition, are defined as “Cluster Consensus Methylation Fraction” (CCMF). See, e.g., FIG. 1B.

CpG clusters are defined as regions of the genome that have a minimum of a specified number of CpG sites (e.g. 4 sites, but could also be 3 or 5, 6, . . . ) within a specified number of bases or less (e.g. 80 bases but could also be smaller or larger). The CpG cluster is defined by the set of CpG sites contained in the cluster. A minimum number of CpG sites per cluster is needed to apply consensus, which is only meaningfully different from existing methods if there is more than one site, and most meaningful if there are more than 2. A specified maximum interval length is needed to ensure that a significant number of reads will cover the whole cluster, which depends on read length and DNA fragment sizes.

### Cell Line Panels

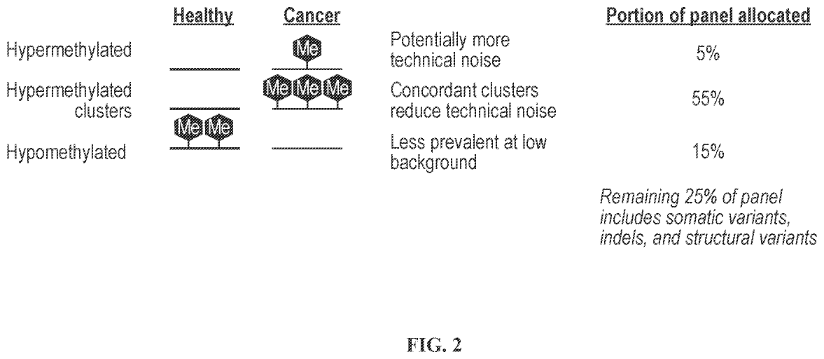

A panel of cell lines was selected for whole-genome methylation sequencing. The panel included one healthy cell line (NA12878) and 4 TNBC cancer cell lines (HCC1187, HCC1937, MDA-MD-453, and BT549). The following features were identified for a ˜200 kb panel. All high confidence short variants in the cancer cell lines were represented, and aberrant methylation loci were prioritized by low signal in background, high signal in cancer cell lines, and CpG density. The portions of the panel allocated to each feature (i.e., hypermethylation, hypermethylated clusters, hypomethylation, somatic variants, indels, and structural variants) are shown in FIG. 2. Cytosine conversion was performed with enzymatic methyl sequencing (EM-seq).

### Results

Methylation data was aggregated across hundreds of selected regions on the panel described above to enable low-level signal detection through a combination of breadth (e.g., number of loci included in the measurement) and depth (e.g., number of independent measurements at each locus). In these experiments, 422 hypermethylated clusters and 156 hypomethylated clusters were analyzed, with an effective 1000× depth of independent measurements at each locus. Data were analyzed according to Average Methylation Fraction (AMF; FIG. 1A) or Cluster Consensus Methylation Fraction (CCMF; FIG. 1B), and the results were compared.

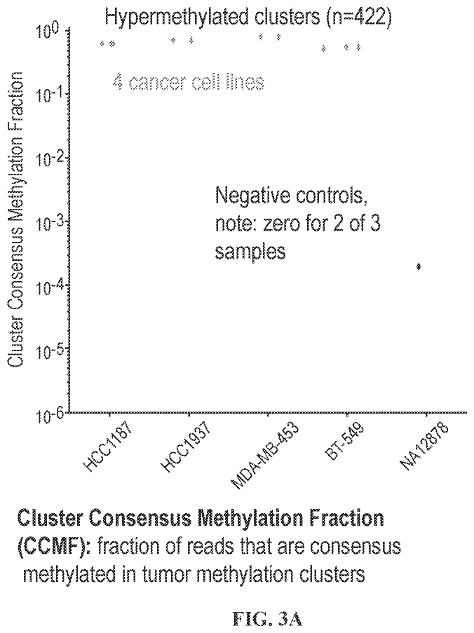

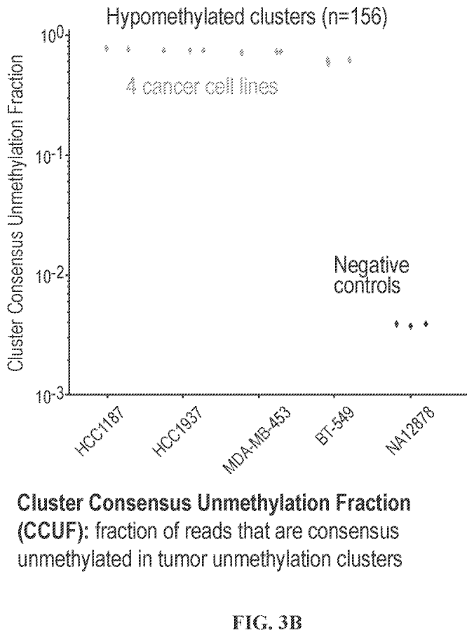

Pure samples showed robust cancer signal above background. The background signal from the negative control (healthy) cell line was lower for CCMF than methylation signal from individual sites (FIG. 3A). CCMF for one negative control sample was found to be 2.1×10⁻⁴, which was likely an outlier, since the CCMF for the remaining 2 negative controls was 0. With an aggregate unique depth of 200-400 k, the true CCMF level was likely less than 10 5. In comparison, the AMF ranged from 7.6-10.2×10⁻⁴. A clear foreground signal was obtained in pure cancer cell line samples, with a CCMF range across cell lines of 0.55-0.81 and a comparable AMF level for the same regions.

Hypomethylated clusters were found to have a higher background signal in the negative controls (FIG. 3B). AMF was calculated to be ˜99%, implying a background level of 1%. CCUF reached only as low as 0.4%. Disparity with hypermethylated clusters could be due to higher biological background or an uncorrected bias or artifact. A clear foreground signal was obtained from the pure cancer cell line samples.

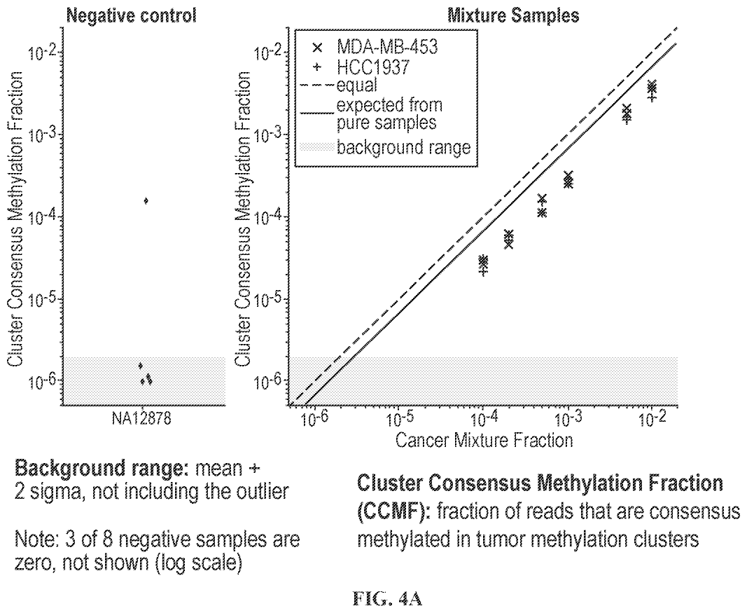

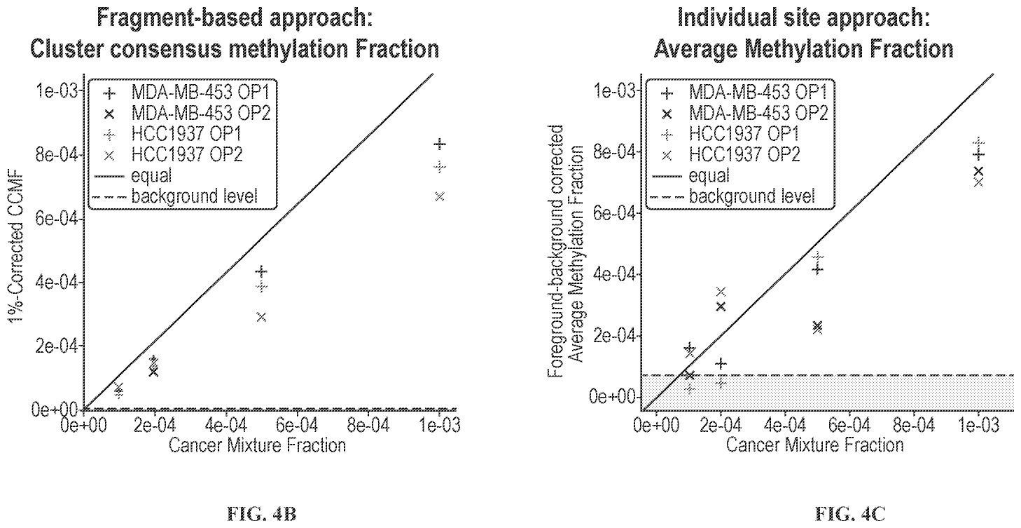

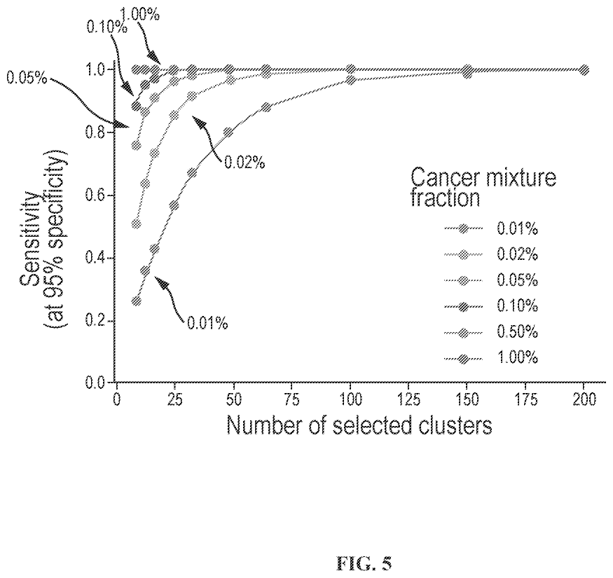

Data from mixture samples demonstrated ultrasensitive detection of methylation. As shown in FIG. 4A, all mixture samples down to 0.01% cancer were found to be well above background range (excluding the single outlier), as analyzed by CCMF. The outlier had a CCMF of 1.6×10⁻⁴, while the other 7 of 8 negative controls were below 2×10⁻⁶. CCMF for mixture samples was found to be consistently below expectation, with CCMF for pure samples having a mean of 0.68 and a range of 0.55-0.81, and the mixture samples having a range of 0.22-0.43 ratio relative to mixture fraction. The fragment-based CCMF approach was found to provide values for mixtures well above background (FIG. 4B), whereas analysis using the individual site-based approach of AMF led to values at or below background for mixtures with a cancer fraction of 2×10⁻⁴ or less (FIG. 4C).

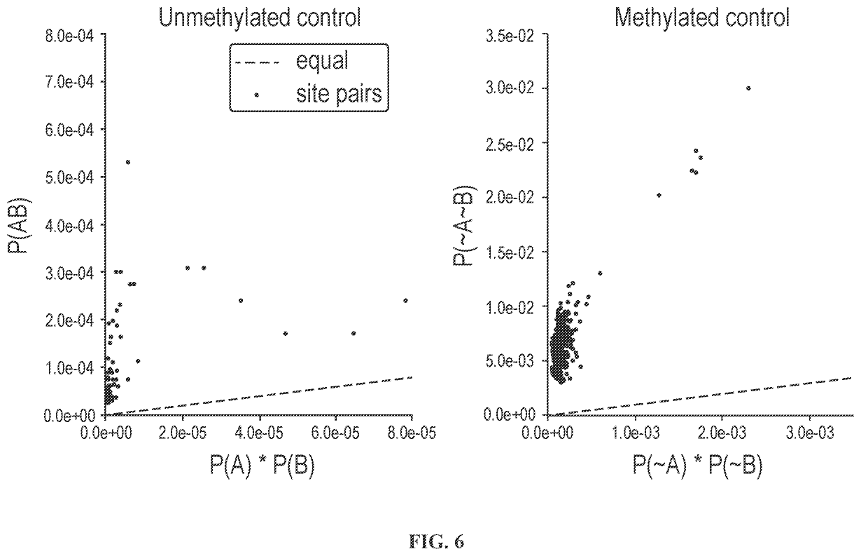

FIG. 5 shows sensitivity (at 95% specificity) of methylation detection by CCMF as a function of the number of clusters selected for analysis, demonstrating ultrasensitive methylation detection. Cancer mixture levels were obtained using laboratory mixtures, not simulations. Data were obtained by sub-sampling hypermethylated clusters from the original set (n=422). These data suggest that, with ˜100 methylation clusters, it would be possible to detect 0.01% cancer mixtures at 95% sensitivity with 95% specificity. With fewer sites, lower detection performance or cancer mixture fractions above 0.01% could be satisfied.

As a complement to aberrant methylation loci, SNPs, indels, and structural variants identified in the pure cancer cell lines were included. This simulates a large set of mutations potentially present at low levels in cfDNA. These analysis included 160 SNPs equally derived from the 4 cell lines of interest, 80 small indels equally derived from the 4 cell lines of interest, and 15 total structural variants (primarily large breakpoint-identified deletions).

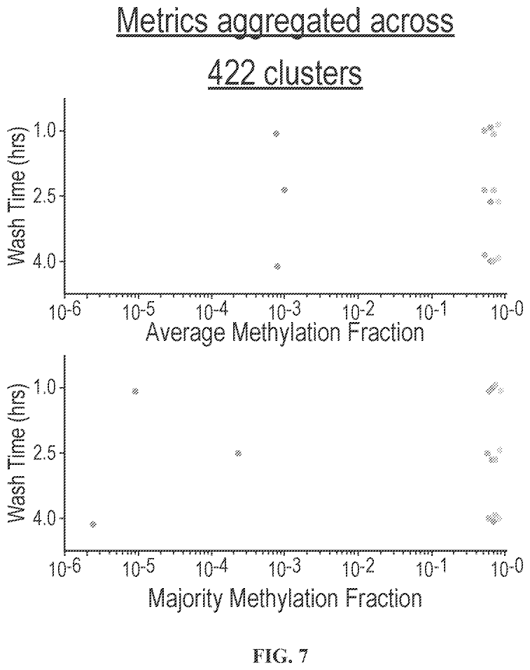

Methylation across a fragment was analyzed in FIG. 6. If aberrant methylation status were linked within fragments, one would expect the methylation at two sites within each fragment to be non-independent:

$$p A \times pB != pAB$$

Data were combined from 18 similar negative control samples to increase aggregate depth. Clusters from chr16 only were used. Aberrant methylation was found to be correlated within fragments in control sample measurements.

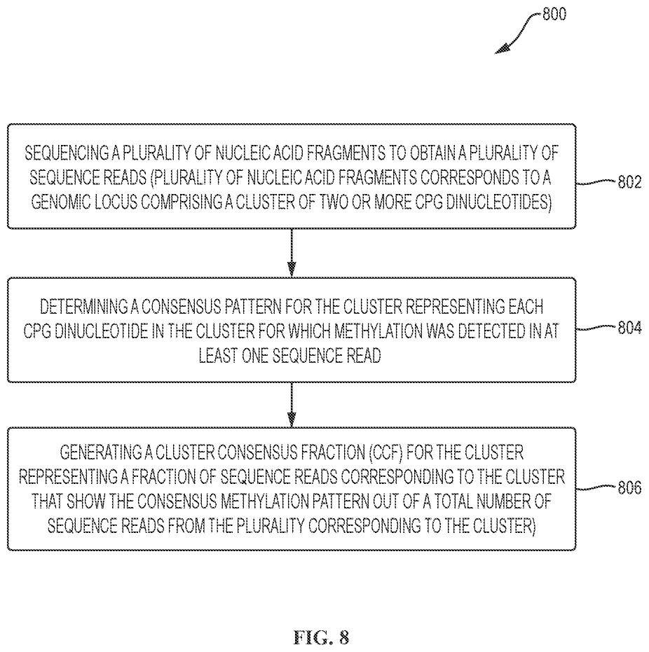

FIG. 7 shows the results from a targeted sequencing experiment. 4 TNBC cancer cell lines were compared to a healthy cell line control. Hybrid capture was applied after cytosine conversion, and different wash times were compared. An average unique target depth of 1000-2000 (lower bound) per sample was achieved, and measurements from each sample represented roughly 200 k-400 k unique reads across 422 regions. AMF and majority methylation fraction (by CCMF) approaches were compared. Both led to robust signal from cancer cell lines, but majority methylation fraction analysis showed values that were up to nearly 3 orders of magnitude lower from healthy cells than those obtained by AMF analysis.

As demonstrated herein, in contrived mixtures of healthy cells and cancer cell lines tested at mixture levels between 0.01% and 1% cancer, the CCMF approach was found to reduce background signal in healthy samples by 100-fold or more. Background level was reduced to below 1 in 100,000. Using the same approach, samples from pure cancer cell lines had signal levels that were similar to the AMF approach or slightly lower. Thus, signal-to-background ratio was effectively increased by more than 100-fold.

At the lowest mixture level tested, cancer samples were clearly distinguishable from negative control samples using the CCMF approach. In contrast, the same analyses carried out with the AMF approach led to indistinguishable measurements between the lower-level mixture samples. Moreover, the CCMF approach led to a measured level in the 0.01% samples that was 10-fold higher than residual background level in the negative control samples, suggesting that even lower mixture levels are likely to be distinguishable.

1. A method of detecting one or more of a methylation level or an unmethylation level of a cluster of two or more CpG dinucleotides in a sample from a subject, comprising:
obtaining a plurality of nucleic acid fragments from the sample;
amplifying the plurality of nucleic acid fragments;
sequencing, by a sequencer, the plurality of amplified nucleic acid fragments to obtain a plurality of sequence reads, wherein at least the plurality of amplified nucleic acid fragments has undergone cytosine conversion, and wherein the plurality of nucleic acid fragments corresponds to a genomic locus comprising a cluster of two or more CpG dinucleotides;
determining, by a processor, a consensus methylation pattern for the cluster, wherein the consensus methylation pattern represents each CpG dinucleotide in the cluster for which methylation was detected based on the cytosine conversion in at least one sequence read from the plurality of sequence reads;
generating, by a processor, a cluster consensus fraction (CCF) for the cluster, wherein the CCF represents a fraction of sequence reads corresponding to the cluster that show the consensus methylation pattern out of a total number of sequence reads from the plurality corresponding to the cluster;
detecting one or more of the methylation level or the unmethylation level of the cluster based on the CCF; and
generating a genomic profile for the subject based on the detected methylation level, the detected unmethylation level, or both.

2. The method of claim 1, wherein:
(a) the CCF is at or above a threshold or reference value, and the method further comprises: detecting presence of cancer nucleic acids in the plurality of nucleic acid fragments, based at least in part on the CCF being at or above the threshold or reference value; or
(b) the CCF is below a threshold or reference value, and the method further comprises: detecting absence of cancer nucleic acids in the plurality of nucleic acid fragments, based at least in part on the CCF being below the threshold or reference value.

3. (canceled)

4. The method of claim 1, comprising determining a consensus methylation pattern and CCF for more than one cluster.

5-11. (canceled)

12. The method of claim 1, wherein at least one CpG dinucleotide in the cluster is unmethylated in the consensus methylation pattern and/or wherein at least one CpG dinucleotide in the cluster is methylated in the consensus methylation pattern.

13-27. (canceled)

28. The method of claim 1, wherein the plurality of sequence reads is obtained from whole-genome methyl sequencing (WGMS) or next-generation sequencing (NGS).

29-40. (canceled)

41. The method of claim 1, wherein the plurality of nucleic acid fragments has undergone cytosine conversion by bisulfite treatment, TET-assisted bisulfite treatment, TET-assisted pyridine borane treatment, oxidative bisulfite treatment, or APOBEC treatment.

42-44. (canceled)

45. The method of claim 1, further comprising, prior to providing the plurality of sequence reads, subjecting a plurality of nucleic acids to fragmentation and/or selectively enriching for a plurality of nucleic acids or nucleic acid fragments corresponding to a genomic locus that comprises a cluster of two or more CpG dinucleotides to produce an enriched sample.

46-47. (canceled)

48. The method of claim 1, further comprising, prior to providing the plurality of sequence reads, isolating the plurality of nucleic acids from the sample.

49-53. (canceled)

54. The method of claim 48, wherein the sample comprises tumor cell-free DNA (cfDNA), circulating cell-free DNA (ccfDNA), or circulating tumor DNA (ctDNA).

55-57. (canceled)

58. The method of claim 1, wherein the sample is a tissue sample, and the method further comprises: subjecting a plurality of nucleic acid molecules in the tissue to fragmentation to create the plurality of nucleic acid fragments.

59. (canceled)

60. A method of detecting cancer in an individual, comprising detecting the methylation level or the unmethylation level according to the method of claim 1 in a sample comprising a plurality of nucleic acids obtained from the individual, wherein the methylation level or the unmethylation level detected in the sample identifies the individual as having cancer.

61. A method of screening an individual suspected of having cancer, comprising detecting the methylation level or the unmethylation level according to the method of claim 1 in a sample comprising a plurality of nucleic acids obtained from the individual, wherein the methylation level or the unmethylation level detected in the sample identifies the individual as likely to have cancer.

62-64. (canceled)

65. A method of predicting tumor burden of an individual having cancer, comprising detecting the methylation level according to the method of claim 1 in a sample comprising a plurality of nucleic acids obtained from the individual, wherein the methylation level or the unmethylation level detected in the sample predicts at least in part the tumor burden of the individual.

66-70. (canceled)

71. A method of treating or delaying progression of cancer, comprising:
(a) detecting the methylation level or the unmethylation level according to the method of claim 1 in a sample comprising a plurality of nucleic acids obtained from the individual, wherein the plurality of nucleic acids includes one or more nucleic acids corresponding to a PITX2 locus; and
(b) administering to the individual an effective amount of anthracycline-based chemotherapy.

72-74. (canceled)

75. A method of treating or delaying progression of cancer, comprising:
(a) detecting the methylation level or the unmethylation level according to the method of claim 1 in a sample comprising a plurality of nucleic acids obtained from the individual, wherein the plurality of nucleic acids includes one or more nucleic acids corresponding to an MGMT locus; and
(b) administering to the individual an effective amount of an alkylating agent.

76-78. (canceled)

79. A method of monitoring a cancer in an individual, comprising:
(a) detecting the methylation level or the unmethylation level according to the method of claim 1 in a first sample comprising a plurality of nucleic acids obtained from the individual;
(b) detecting the methylation level or the unmethylation level according to the method of claim 1 in a second sample comprising a plurality of nucleic acids obtained from the individual, wherein the second sample is obtained from the individual after the first sample; and
(c) determining a difference in methylation level between the first and second samples, thereby monitoring the cancer in the individual.

80. A method of monitoring response of an individual being treated for cancer, comprising:
(a) detecting the methylation level or the unmethylation level according to the method of claim 1 in a first sample comprising a plurality of nucleic acids obtained from the individual;
(b) after the first sample is obtained from the individual, administering a treatment to the individual;
(c) detecting the methylation level or the unmethylation level according to the method of any one of claims 1-59 in a second sample comprising a plurality of nucleic acids obtained from the individual, wherein the second sample is obtained from the individual after administration of the treatment; and
(d) determining a difference in methylation level between the first and second samples, thereby monitoring response of the individual to the treatment.

81. A method of detecting one or more of a methylation level or an unmethylation level of a cluster of two or more CpG dinucleotides from a sample, comprising:
obtaining a plurality of sequence reads from a plurality of nucleic acid fragments exhibiting cytosine conversion;
determining, by a processor, a consensus methylation pattern for a cluster of two or more CpG dinucleotides at a locus, wherein the consensus methylation pattern represents each CpG dinucleotide in the cluster for which methylation was detected;
generating, by a processor, a cluster consensus fraction (CCF) for the cluster, wherein the CCF represents a fraction of sequence reads corresponding to the cluster that show the consensus methylation pattern out of a total number of sequence reads from the plurality corresponding to the cluster; and
detecting, by the processor, one or more of the methylation level or the unmethylation level of the cluster based on the CCF.

82. (canceled)

83. The method of claim 81, wherein the consensus methylation pattern represents each CpG dinucleotide in the cluster for which methylation was detected based on the cytosine conversion in at least one sequence read from the plurality.

84-226. (canceled)

227. A method of detecting one or more of a methylation level or an unmethylation level of a cluster of two or more CpG dinucleotides in a sample from a subject, comprising:
obtaining a plurality of nucleic acid fragments from the sample;
amplifying the plurality of nucleic acid fragments;
sequencing, by a sequencer, the plurality of amplified nucleic acid fragments to obtain a plurality of sequence reads, wherein at least the plurality of amplified nucleic acid fragments has undergone cytosine conversion, and wherein the plurality of nucleic acid fragments corresponds to a genomic locus comprising a cluster of two or more CpG dinucleotides;
determining, by a processor, a consensus unmethylation pattern for the cluster, wherein the consensus unmethylation pattern represents each CpG dinucleotide in the cluster for which methylation was not detected based on the cytosine conversion in at least one sequence read from the plurality of sequence reads;
generating, by a processor, a cluster consensus fraction (CCF) for the cluster, wherein the CCF represents a fraction of sequence reads corresponding to the cluster that show the consensus unmethylation pattern out of a total number of sequence reads from the plurality corresponding to the cluster;
detecting one or more of the methylation level or the unmethylation level of the cluster based on the CCF; and
generating a genomic profile for the subject based on the detected methylation level, the detected unmethylation level, or both.

228-373. (canceled)
